SEQUENCE LISTING (120 sequences)

SEQ ID NO: 1 - DNA, 40 bp, Homo sapiens
tatgacgccacgccaaggagggaagagtcccagtgaactc

SEQ ID NO: 2 - DNA, 40 bp, Homo sapiens
ccttgtggaatgcagctgacccagctgatagaggaagtag

SEQ ID NO: 3 - DNA, 40 bp, Homo sapiens
aagctgctactcctagtaggccaaacgctcaggttaaaca

SEQ ID NO: 4 - DNA, 40 bp, Homo sapiens
agggaccttggaaacagttggcactgtaaggtgcttgctc

SEQ ID NO: 5 - DNA, 40 bp, Homo sapiens
agctggcactagtcaagatggaggtgatgctggtacttca

SEQ ID NO: 6 - DNA, 40 bp, Homo sapiens
ggcgcaatacaccgcttctgggtcaggagttagaagctac

SEQ ID NO: 7 - DNA, 40 bp, Homo sapiens
catcccaagacagccagcaggttgtctctggaaacgacca

SEQ ID NO: 8 - DNA, 40 bp, Homo sapiens
cacgtgctgctgacaccgaccggagtactagccagcacaa

SEQ ID NO: 9 - DNA, 40 bp, Homo sapiens
gctggacactgtccatgaagcactgagcagaagctggagg

SEQ ID NO: 10 - DNA, 40 bp, Homo sapiens
tagtgaggatagttctagtgactcagaagatgaagattgc

SEQ ID NO: 11 - DNA, 40 bp, Homo sapiens
tatagggtgtttgtagaagggataaatgggttacctaatg

SEQ ID NO: 12 - DNA, 40 bp, Homo sapiens
actgccaaaatacattgaccgtagtagctgttcaactcct

SEQ ID NO: 13 - DNA, 40 bp, Homo sapiens
ttgaagagatgcaagagggcccagtgaggacatccgcctc

SEQ ID NO: 14 - DNA, 40 bp, Homo sapiens
ataagagtgacttgaccaaggacgtcacaacatcagtgct

SEQ ID NO: 15 - DNA, 40 bp, Homo sapiens
tcctgtgtctacattacaacgtaccaatccaagaaagcaa

SEQ ID NO: 16 - DNA, 40 bp, Homo sapiens
aatcctttaaggagatcgtcgtcactgactactcagacca

SEQ ID NO: 17 - DNA, 40 bp, Homo sapiens
gcagatcccaatcaaagtgccaagtgagcttgaagttgga

SEQ ID NO: 18 - DNA, 40 bp, Homo sapiens
catttgatgctggtagtatggattatgagatggactagcc

SEQ ID NO: 19 - DNA, 40 bp, Homo sapiens
tacacatgtaagttgtatggcagtttacagaactcaatga

SEQ ID NO: 20 - DNA, 40 bp, Homo sapiens
gtgtgtcctgaacagtgtagctcaggtcagcttgaacttt

SEQ ID NO: 21 - DNA, 40 bp, Homo sapiens
ctagaatccttggttcactgctgtcttcatgtgttctatg

SEQ ID NO: 22 - DNA, 40 bp, Homo sapiens
agagggtatggttaggtacgggtcttcctgcctcattcct

SEQ ID NO: 23 - DNA, 40 bp, Homo sapiens
ttcggagcctgccacccaggccctcagaactgagccacag

SEQ ID NO: 24 - DNA, 40 bp, Homo sapiens
ccctttcagatgtcttctgcctgttataactctgcactac

SEQ ID NO: 25 - DNA, 40 bp, Homo sapiens
tcacattgaacagctgtgagacagacatattgagatgcct

SEQ ID NO: 26 - DNA, 40 bp, Homo sapiens
tgccaggtcagatggagacgcagaacctgctggtgcaagc

SEQ ID NO: 27 - DNA, 40 bp, Homo sapiens
aggtctgtgtaaagtaaggggagtgttaggagcagccagg

SEQ ID NO: 28 - DNA, 40 bp, Homo sapiens
tgagttactaactaaccacgcgtgttgttcctatgtgctt

SEQ ID NO: 29 - DNA, 40 bp, Homo sapiens
aggttaattgatagacccaccacctcttgcactctcgctt

SEQ ID NO: 30 - DNA, 40 bp, Homo sapiens
gtcagaagtgcagaattggggcacttaatggtcaccttgt

SEQ ID NO: 31 - DNA, 64 bp, Homo sapiens
taatacgactcactatagggtacaaatcatcaatcactttaatctatgacgccacgccaaggag

SEQ ID NO: 32 - DNA, 64 bp, Homo sapiens
taatacgactcactatagggtacactttctttctttctttctttccttgtggaatgcagctgac

SEQ ID NO: 33 - DNA, 64 bp, Homo sapiens
taatacgactcactatagggcaataaactatacttcttcactaaaagctgctactcctagtagg

SEQ ID NO: 34 - DNA, 64 bp, Homo sapiens
taatacgactcactatagggctactatacatcttactatactttagggaccttggaaacagttg

SEQ ID NO: 35 - DNA, 64 bp, Homo sapiens
taatacgactcactatagggatacttcattcattcatcaattcaagctggcactagtcaagatg

SEQ ID NO: 36 - DNA, 64 bp, Homo sapiens
taatacgactcactatagggaatcaatcttcattcaaatcatcagtgtactactcccagactcc

SEQ ID NO: 37 - DNA, 64 bp, Homo sapiens
taatacgactcactatagggaatcctttctttaatctcaaatcacatcccaagacagccagcag

SEQ ID NO: 38 - DNA, 64 bp, Homo sapiens
taatacgactcactatagggttcaatcattcaaatctcaactttcacgtgctgctgacaccgac

SEQ ID NO: 39 - DNA, 64 bp, Homo sapiens
taatacgactcactatagggtcaattaccttttcaatacaatacgctggacactgtccatgaag

SEQ ID NO: 40 - DNA, 64 bp, Homo sapiens
taatacgactcactatagggcttttcaattacttcaaatcttcatagtgaggatagttctagtg

SEQ ID NO: 41 - DNA, 64 bp, Homo sapiens
taatacgactcactatagggttactcaaaatctacactttttcatatagggtgtttgtagaagg

SEQ ID NO: 42 - DNA, 64 bp, Homo sapiens
taatacgactcactatagggcttttcaaatcaatactcaactttactgccaaaatacattgacc

SEQ ID NO: 43 - DNA, 64 bp, Homo sapiens
taatacgactcactatagggaatcttactacaaatcctttctttttgaagagatgcaagagggc

SEQ ID NO: 44 - DNA, 64 bp, Homo sapiens
taatacgactcactatagggcaatttcatcattcattcatttcaataagagtgacttgaccaag

SEQ ID NO: 45 - DNA, 64 bp, Homo sapiens
taatacgactcactatagggcttttcatcttttcatctttcaattcctgtgtctacattacaac

SEQ ID NO: 46 - DNA, 64 bp, Homo sapiens
taatacgactcactatagggtcaatcattacacttttcaacaataatcctttaaggagatcgtc

SEQ ID NO: 47 - DNA, 64 bp, Homo sapiens
taatacgactcactatagggtacacaatcttttcattacatcatgcagatcccaatcaaagtgc

SEQ ID NO: 48 - DNA, 64 bp, Homo sapiens
taatacgactcactatagggctttctacattattcacaacattacatttgatgctggtagtatg

SEQ ID NO: 49 - DNA, 64 bp, Homo sapiens
taatacgactcactatagggctatcttcatatttcactataaactacacatgtaagttgtatgg

SEQ ID NO: 50 - DNA, 64 bp, Homo sapiens
taatacgactcactatagggtcatttcacaattcaattactcaagtgtgtcctgaacagtgtag

SEQ ID NO: 51 - DNA, 64 bp, Homo sapiens
taatacgactcactatagggcttctcattaacttacttcataatctagaatccttggttcactg

SEQ ID NO: 52 - DNA, 64 bp, Homo sapiens
taatacgactcactatagggaaacaaacttcacatctcaataatagagggtatggttaggtacg

SEQ ID NO: 53 - DNA, 64 bp, Homo sapiens
taatacgactcactatagggtcatcaatctttcaatttacttacttcggagcctgccacccagg

SEQ ID NO: 54 - DNA, 64 bp, Homo sapiens
taatacgactcactatagggcaatataccaatatcatcatttacccctttcagatgtcttctgc

SEQ ID NO: 55 - DNA, 64 bp, Homo sapiens
taatacgactcactatagggtcatttcaatcaatcatcaacaattcacattgaacagctgtgag

SEQ ID NO: 56 - DNA, 64 bp, Homo sapiens
taatacgactcactatagggtaattatacatctcatcttctacatgccaggtcagatggagacg

SEQ ID NO: 57 - DNA, 64 bp, Homo sapiens
taatacgactcactatagggctattacactttaaacatcaatacaggtctgtgtaaagtaaggg

SEQ ID NO: 58 - DNA, 64 bp, Homo sapiens
taatacgactcactatagggctatctatctaactatctatatcatgagttactaactaaccacg

SEQ ID NO: 59 - DNA, 64 bp, Homo sapiens
taatacgactcactatagggaatctacactaacaatttcataacaggttaattgatagacccac

SEQ ID NO: 60 - DNA, 64 bp, Homo sapiens
taatacgactcactatagggctatctttaaactacaaatctaacgtcagaagtgcagaattggg

SEQ ID NO: 61 - DNA, 40 bp, Homo sapiens
ggaagagtcccagtgaactctccctttagtgagggttaat

SEQ ID NO: 62 - DNA, 40 bp, Homo sapiens
ccagctgatagaggaagtagtccctttagtgagggttaat

SEQ ID NO: 63 - DNA, 40 bp, Homo sapiens
ccaaacgctcaggttaaacatccctttagtgagggttaat

SEQ ID NO: 64 - DNA, 40 bp, Homo sapiens
gcactgtaaggtgcttgctctccctttagtgagggttaat

SEQ ID NO: 65 - DNA, 40 bp, Homo sapiens
gaggtgatgctggtacttcatccctttagtgagggttaat

SEQ ID NO: 66 - DNA, 40 bp, Homo sapiens
gtacgccttcacgtcctccttccctttagtgagggttaat

SEQ ID NO: 67 - DNA, 40 bp, Homo sapiens
gttgtctctggaaacgaccatccctttagtgagggttaat

SEQ ID NO: 68 - DNA, 40 bp, Homo sapiens
cggagtactagccagcacaatccctttagtgagggttaat

SEQ ID NO: 69 - DNA, 40 bp, Homo sapiens
cactgagcagaagctggaggtccctttagtgagggttaat

SEQ ID NO: 70 - DNA, 40 bp, Homo sapiens
actcagaagatgaagattgctccctttagtgagggttaat

SEQ ID NO: 71 - DNA, 40 bp, Homo sapiens
gataaatgggttacctaatgtccctttagtgagggttaat

SEQ ID NO: 72 - DNA, 40 bp, Homo sapiens
gtagtagctgttcaactccttccctttagtgagggttaat

SEQ ID NO: 73 - DNA, 40 bp, Homo sapiens
ccagtgaggacatccgcctctccctttagtgagggttaat

SEQ ID NO: 74 - DNA, 40 bp, Homo sapiens
gacgtcacaacatcagtgcttccctttagtgagggttaat

SEQ ID NO: 75 - DNA, 40 bp, Homo sapiens
gtaccaatccaagaaagcaatccctttagtgagggttaat

SEQ ID NO: 76 - DNA, 40 bp, Homo sapiens
gtcactgactactcagaccatccctttagtgagggttaat

SEQ ID NO: 77 - DNA, 40 bp, Homo sapiens
caagtgagcttgaagttggatccctttagtgagggttaat

SEQ ID NO: 78 - DNA, 40 bp, Homo sapiens
gattatgagatggactagcctccctttagtgagggttaat

SEQ ID NO: 79 - DNA, 40 bp, Homo sapiens
cagtttacagaactcaatgatccctttagtgagggttaat

SEQ ID NO: 80 - DNA, 40 bp, Homo sapiens
ctcaggtcagcttgaacttttccctttagtgagggttaat

SEQ ID NO: 81 - DNA, 40 bp, Homo sapiens
ctgtcttcatgtgttctatgtccctttagtgagggttaat

SEQ ID NO: 82 - DNA, 40 bp, Homo sapiens
ggtcttcctgcctcattccttccctttagtgagggttaat

SEQ ID NO: 83 - DNA, 40 bp, Homo sapiens
ccctcagaactgagccacagtccctttagtgagggttaat

SEQ ID NO: 84 - DNA, 40 bp, Homo sapiens
ctgttataactctgcactactccctttagtgagggttaat

SEQ ID NO: 85 - DNA, 40 bp, Homo sapiens
acagacatattgagatgccttccctttagtgagggttaat

SEQ ID NO: 86 - DNA, 40 bp, Homo sapiens
cagaacctgctggtgcaagctccctttagtgagggttaat

SEQ ID NO: 87 - DNA, 40 bp, Homo sapiens
gagtgttaggagcagccaggtccctttagtgagggttaat

SEQ ID NO: 88 - DNA, 40 bp, Homo sapiens
cgtgttgttcctatgtgctttccctttagtgagggttaat

SEQ ID NO: 89 - DNA, 40 bp, Homo sapiens
cacctcttgcactctcgctttccctttagtgagggttaat

SEQ ID NO: 90 - DNA, 40 bp, Homo sapiens
gcacttaatggtcaccttgttccctttagtgagggttaat

SEQ ID NO: 91 - DNA, 24 bp, Homo sapiens
gattaaagtgattgatgatttgta

SEQ ID NO: 92 - DNA, 24 bp, Homo sapiens
aaagaaagaaagaaagaaagtgta

SEQ ID NO: 93 - DNA, 24 bp, Homo sapiens
ttagtgaagaagtatagtttattg

SEQ ID NO: 94 - DNA, 24 bp, Homo sapiens
aaagtatagtaagatgtatagtag

SEQ ID NO: 95 - DNA, 24 bp, Homo sapiens
tgaattgatgaatgaatgaagtat

SEQ ID NO: 96 - DNA, 24 bp, Homo sapiens
tgatgatttgaatgaagattgatt

SEQ ID NO: 97 - DNA, 24 bp, Homo sapiens
tgatttgagattaaagaaaggatt

SEQ ID NO: 98 - DNA, 24 bp, Homo sapiens
aaagttgagatttgaatgattgaa

SEQ ID NO: 99 - DNA, 24 bp, Homo sapiens
gtattgtattgaaaaggtaattga

SEQ ID NO: 100 - DNA, 24 bp, Homo sapiens
tgaagatttgaagtaattgaaaag

SEQ ID NO: 101 - DNA, 24 bp, Homo sapiens
tgaaaaagtgtagattttgagtaa

SEQ ID NO: 102 - DNA, 24 bp, Homo sapiens
aaagttgagtattgatttgaaaag

SEQ ID NO: 103 - DNA, 24 bp, Homo sapiens
aaagaaaggatttgtagtaagatt

SEQ ID NO: 104 - DNA, 24 bp, Homo sapiens
tgaaatgaatgaatgatgaaattg

SEQ ID NO: 105 - DNA, 24 bp, Homo sapiens
attgaaagatgaaaagatgaaaag

SEQ ID NO: 106 - DNA, 24 bp, Homo sapiens
attgttgaaaagtgtaatgattga

SEQ ID NO: 107 - DNA, 24 bp, Homo sapiens
atgatgtaatgaaaagattgtgta

SEQ ID NO: 108 - DNA, 24 bp, Homo sapiens
taatgttgtgaataatgtagaaag

SEQ ID NO: 109 - DNA, 24 bp, Homo sapiens
gtttatagtgaaatatgaagatag

SEQ ID NO: 110 - DNA, 24 bp, Homo sapiens
ttgagtaattgaattgtgaaatga

SEQ ID NO: 111 - DNA, 24 bp, Homo sapiens
attatgaagtaagttaatgagaag

SEQ ID NO: 112 - DNA, 24 bp, Homo sapiens
attattgagatgtgaagtttgttt

SEQ ID NO: 113 - DNA, 24 bp, Homo sapiens
gtaagtaaattgaaagattgatga

SEQ ID NO: 114 - DNA, 24 bp, Homo sapiens
gtaaatgatgatattggtatattg

SEQ ID NO: 115 - DNA, 24 bp, Homo sapiens
attgttgatgattgattgaaatga

SEQ ID NO: 116 - DNA, 24 bp, Homo sapiens
tgtagaagatgagatgtataatta

SEQ ID NO: 117 - DNA, 24 bp, Homo sapiens
gtattgatgtttaaagtgtaatag

SEQ ID NO: 118 - DNA, 24 bp, Homo sapiens
tgatatagatagttagatagatag

SEQ ID NO: 119 - DNA, 24 bp, Homo sapiens
gttatgaaattgttagtgtagatt

SEQ ID NO: 120 - DNA, 24 bp, Homo sapiens
gttagatttgtagtttaaagatag

(19) United States
(12) Patent Application Publication
(10) Pub. No.: US 2011/0263693 A1
(43) Pub. Date: Oct. 27, 2011

(54) CELASTROL, GEDUNIN, AND DERIVATIVES THEREOF AS HSP90 INHIBITORS

(75) Inventors: Haley Vinson-Hieronymus, Brooklyn, NY (US); Todd R. Golub, Newton, MA (US); Justin Lamb, Cambridge, MA (US); Kimberly Stegmaier, Jamaica Plain, MA (US)

(73) Assignees: DANA-FARBER CANCER INSTITUTE, INC., Boston, MA (US); MASSACHUSETTS INSTITUTE OF TECHNOLOGY, Cambridge, MA (US)

(21) Appl. No.: 12/294,507

(22) PCT Filed: Mar. 30, 2007
(86) PCT No.: PCT/US2007/008308

§ 371 Date: Jul. 6, 2011

Related U.S. Application Data
(60) Provisional application No. 60/787,895, filed on Mar. 31, 2006.

(51) Int. Cl. A61K 31/352; A61K 31/225; A61K 31/19; A61P 35/00; C07D 311/78; C07C 69/604; C07C 63/44; A61P 35/02; C12N 5/09; C07D 493/04

(52) U.S. Cl. 514/453; 435/375; 514/548; 514/569; 549/275; 549/277; 560/194; 562/405

(57) ABSTRACT

Based on the discovery that celastrol and gedunin are Hsp90 inhibitors, the present invention provides novel inhibitors of Hsp90. and pharmaceutically acceptable salts, derivatives, and compositions thereof. The invention provides two classes of compounds. One class includes celastrol and its derivatives. The other class includes gedunin and its derivatives. The present invention further provides methods for treating disorders wherein Hsρ90 inhibition is desired (e.g., proliferative diseases, cancer, inflammatory diseases, fungal infections, etc.) comprising administering a therapeutically effective amount of an inventive compound to a subject in need thereof. Celastrol, gedunin, and derivatives thereof are particularly useful in the treatment of prostate cancer, breast cancer, ovarian cancer, lung cancer, and leukemia.

### GOVERNMENT SUPPORT

The work described herein was supported, in part, by grants from the National Institutes of Health (5P50 CA090381). The United States government may have certain rights in the invention.

### BACKGROUND OF THE INVENTION

The heat shock proteins, including Hsp90, mediate the folding, stability, activation, and degradation of many key cellular regulators and receptors. They thereby play an important role in cell signaling, growth, and survival. For a general review of heat shock proteins, see Parsell and Lindquist, Ann. Rev. Genet. 27:437-496, 1993; incorporated herein by reference. The Hsp90 family of heat shock proteins is a group of highly conserved stress proteins that are expressed in all eukaryotic cells. Hsp90 is an ATP-dependent chaperone belonging to the ATPase/kinase superfamily bearing a Bergerat ATP-binding fold. Dutta et al. Trends Biochem. Sci. 25:24-28, 2000; Terasawa et al. J. Biochem. 137:443-447, 2005; each of which is incorporated herein by reference. Hsp90 is one of the most abundant proteins in eukaryotic cells, constituting up to about 1-2% of the total cellular protein under normal physiologic conditions. Its expression is increased several-fold in response to stress. In most eukaryotic cells, one of two Hsp90 family members is expressed constitutively at a high level at physiological temperature and is induced only 2-3 times by heat shock. A second family member is expressed at a low basel level at normal temperatures, but its expression is enhanced strongly under restrictive growth conditions, like heat treatment. Borkovich et al. Mol. Cell. Biol. 9:3919-3930, 1989; Krone and Sass, Biochem Biophys. Res. Commun. 204:746-752, 1994; each of which is incorporated herein by reference.

The two genes that encode Hsp90 in humans are Hsp90α and Hsp90β. These proteins are 86% homologous. Furthermore, there is extensive homology with lower species. The 63 kDa Hsp90 homolog in E. coli is 42% identical in amino acid sequence to human Hsp90. And the 83 kDa Hsp90 protein homolog of Drosophila is 78% identical to human Hsp90. Alique et al. EMBO J. 13:6099-6106, 1994; Rebbe et al. Gene 53:235-245, 1987; Blackman et al., J. Mol. Biol. 188:499-515, 1986; each of which is incorporated herein by reference.

The Hsp90 family has been implicated as an important component of intracellular signaling pathways as well as in assisting protein folding. More than 40 proteins are clients of the Hsp90α and Hsp90β isoforms and have been reviewed. Richter et al. J. Cell. Physiol. 188:281-290, 2001; Maloney et al. Expert Opin. Biol. Ther. 2:3-24, 2002; Dai et al. Future Oncol. 1:529-540, 2005; each of which is incorporated herein by reference. Dimeric Hsp90 proteins bind molecules such as steroid hormone receptors and the receptor kinases, v-src, Raf, and casein kinase II. Catelli et al. EMBO J. 4:3131-3135, 1985; Miyata and Yahara, J. Biol. Chem. 267:7042-7047, 1992; Stancato et al., J. Biol. Chem. 268:21711-21716, 1993; Xu and Lindquist, Proc. Natl. Acad. Sci. USA 90:7074-7078, 1993; Wartmann and Davis, J. Biol. Chem. 269:6695-6701, 1994; van der Straten et al., EMBO J. 16:1961-1969, 1997; each of which is incorporated herein by reference. In the case of steroid receptors, this interaction is required for efficient ligand binding and transcriptional regulation. Bohen and Yamamoto, “Modulation of Steroid Receptor Signal Transduction by Heat Shock Proteins” In: The Biology of Heat Shock Proteins and Molecular Chaperones, Cold Spring Harbor Laboratory Press, pp. 313-334, 1994.

Hsp90 inhibitors have been found useful as cancer therapies, for example, geldanamycin and 17-AAG. Currently, 17-AAG, an Hsp90 inhibitor, has been tested in a number of phase I clinical trials, and a number of phase II trials are ongoing. Both existing and novel Hsp90 inhibitors are of notable interest because of their ability to act on multiple oncogenic pathways. Cancer cells have also been reported to be more sensitive to Hsp90 inhibition than non-malignant cells due to increased intracellular Hsp90 inhibitor levels and increased sensitivity of oncogenic mutants of key proteins. Pre-clinical studies have demonstrated the role of Hsp90 inhibitors in the treatment of cancers, including prostate cancer, leukemia, lung cancer, breast cancer, ovarian cancer, and others, and in the treatment of infectious diseases such as fungal infections.

### SUMMARY OF THE INVENTION

Celastrol, gedunin, and derivatives thereof have been found to inhibit heat shock protein 90 (Hsp90). Celastrol and gedunin represent novel classes of Hsp90 inhibitors, and like other Hsp90 inhibitors are useful in the treatment of cancer. These compounds are structurally distinct from existing Hsp90 inhibitors and may act via a different mechanism than existing Hsp90 inhibitors. Therefore, existing Hsp90 inhibitors may act synergistically with celastrol, gedunin, and derivatives thereof as described herein. These compounds may also be combined with more traditional chemotherapeutic agents in the treatment of cancer. These new classes of Hsp90 inhibitors may also find use in the treatment of other Hsp90-dependent conditions. For example, these compounds may be useful in the treatment of infectious diseases such as fungal infections.

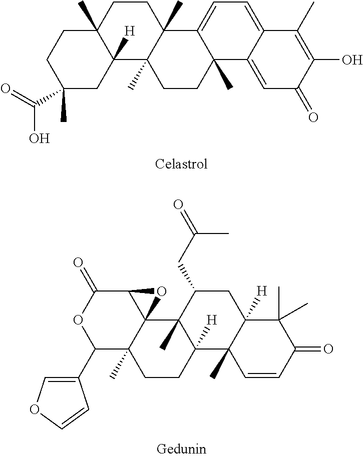

In certain embodiments, celastrol, gedunin, or derivates thereof are useful in accordance with the present invention. Particular exemplary derivatives of celastrol that are useful in the present invention include compounds of the formula:

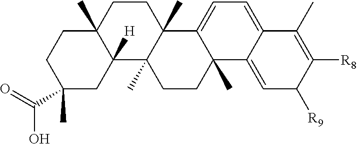

wherein

R₈ is hydroxyl (—OH) or acetyl-protected hydroxyl

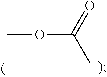

and

R₉ is oxo (═O), hydrogen (—H), or acetyl-protected hydroxyl

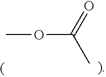

Particular exemplary derivatives of gedunin that are useful in the present invention include compounds of the formula:

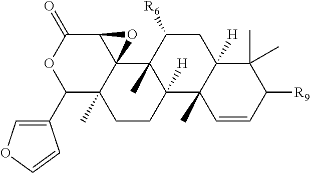

wherein

R₆ is hydrogen (—H); oxo (═O), hydroxyl (—OH), or acetyl-protected hydroxyl

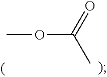

and

R₉ is oxo (═O), or acetyl-protected hydroxyl

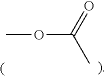

The present invention provides two novel classes of inhibitors of Hsp90. One class, of which celastrol is a member, include compounds of formula:

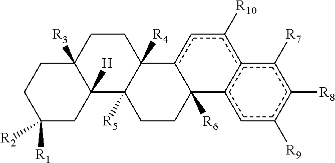

wherein

each dashed line independently represents either the presence or absence of a bond;

R₁ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OFT; —OR_{A}; —C(═O)R_{A}; —CHO; —CO₂H; —CO₂R_{A}; —CN; —SCN; —SR_{A}; —SOR_{A}; —SO₂R_{A}; —NO₂; —N₃; —NH₂; —NHR_{A}; —N(R_{A})₂; —NHC(═O)R_{A}; —NR_{A}C(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₂ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{B}; —C(═O)R_{B}; —CHO; —CO₂H; —CO₂R_{B}; —CN; —SCN; —SR_{B}; —SOR_{B}; —SO₂R_{B}; —NO₂; —N₃; —NH₂; —NHR_{B}; —N(R_{B})₂; —NHC(═O)R_{B}; —NR_{B}C(═O)R_{B}; —NR_{B}C(═O)N(R_{B})₂; —OC(═O)OR_{B}; —OC(═O)R_{B}; OC(═O)N(R_{B})₂; —NR_{B}C(═O)OR_{B}; or —C(R_{B})₃; wherein each occurrence of R_{B} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₃ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{C}; —C(═O)R_{C}; CHO; —CO₂H; —CO₂R_{C}; —CN; —SCN; —SR_{C}; —SOR_{C}; —SO₂R_{C}; —NO₂; —N₃; —NH₂; —NHR_{C}; —N(R_{C})₂; —NHC(═O)R_{C}; —NR_{C}C(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₄ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{D}; —C(═O)R_{D}; —CHO; —CO₂H; —CO₂R_{D}; —CN; —SCN; —SR_{D}; —SOR_{D}; —SO₂R_{D}; —NO₂; —N₃; —NH₂; —NHR_{D}; —N(R_{D})₂; —NHC(═O)R_{D}; —NR_{D}C(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₅ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{E}; —C(═O)R_{E}; —CHO; —CO₂H; —CO₂R_{E}; —CN; —SCN; —SR_{E}; —SOR_{E}; —SO₂R_{E}; —NO₂; —N₃; —NH₂; —NHR_{E}; —N(R_{E})₂; —NHC(═O)R_{E}; —NR_{E}C(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₆ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{F}; —C(═O)R_{F}; —CHO; —CO₂H; —CO₂R_{F}; —CN; —SCN; —SR_{F}; —SOR_{E}; —SO₂R_{F}; —NO₂; —N₃; —NH₂; —NHR_{F}; —N(R_{F})₂; —NHC(═O)R_{F}; —NR_{F}C(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₇ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{G}; ═O; —C(═O)R_{G}; —CHO; —CO₂H; —CO₂R_{G}; —CN; —SCN; —SR_{G}; —SOR_{G}; —SO₂R_{G}; —NO₂; —N₃; —NH₂; —NHR_{G}; —N(R_{G})₂; —NHC(═O)R_{G}; —NR_{G}C(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₈ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{H}; ═O; —C(═O)R_{H}; —CHO; —CO₂H; —CO₂R_{H}; —CN; —SCN; —SR_{H}; —SOR_{H}; —SO₂R_{H}; —NO₂; —N₃; —NH₂; —NHR_{H}; —N(R_{H})₂; —NHC(═O)R_{H}; —NR_{H}C(═O)R_{H}; —NR_{H}C(═O)N(R_{H})₂; —OC(═O)OR_{H}; —OC(═O)N(R_{H})₂; —NR_{H}C(═O)OR_{H}; or —C(R_{H})₃; wherein each occurrence of R_{H} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₉ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{I}; ═O; —C(═O)R_{I}; —CHO; —CO₂H; —CO₂R_{I}; —CN; —SCN; —SR_{I}; —SO₂R_{I}; —SO₂R_{I}; —NO₂; —N₃; —NH₂; —NHR_{I}; —N(R_{I})₂; —NHC(═O)R_{I}; —NR_{I}C(═O)R_{I}; —NR_{I}C(═O)N(R_{I})₂; —OC(═O)OR_{I}; —OC(═O)R_{I}; —OC(═O)N(R_{I})₂; —NR_{I}C(═O)OR_{I}; or —C(R_{I})₃; wherein each occurrence of R₁ is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₁₀ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{J}; ═O; —C(═O)R_{J}; —CHO; —CO₂H; —CO₂R_{J}; —CN; —SCN; —SR_{J}; —SOR_{J}; —SO₂R_{J}; —NO₂; —N₃; —NH₂; —NHR_{I}; —N(R_{J})₂; —NHC(═O)R_{J}; —NR_{J}C(═O)R_{J}; —NR_{J}C(═O)N(R_{J})₂; —OC(═O)OR_{J}; —OC(═O)R_{J}; —OC(═O)N(R_{J})₂; —NR_{I}C(═O)OR_{J}; or —C(R_{J})₃; wherein each occurrence of R_{J} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety; and pharmaceutically acceptable salts, stereoisomers, tautomers, and pro-drugs thereof.

The invention also provides a second class of Hsp90 inhibitors, of which gedunin is a member. This second class includes compounds of formula:

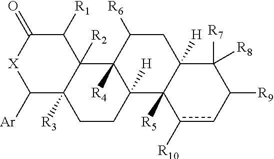

wherein

Ar is a substituted or unsubstituted aryl or heteroaryl moiety;

X is —O—, —NH—, —NR_{X}—, —CH₂—, —CHR_{X}—, or —C(R_{X})₂—, wherein R_{X} is a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; heteroaryloxy; or heteroarylthio moiety;

a dashed line represents either the presence or absence of a bond;

R₁ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{A}; —C(═O)R_{A}; —CHO; —CO₂H; —CO₂R_{A}; —CN; —SCN; —SR_{A}; —SOR_{A}; —SO₂R_{A}; —NO₂; —N₃; —NH₂; —NHR_{A}; —N(R_{A})₂; —NHC(═O)R_{A}; —NR_{A}C(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₂ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{B}; —C(═O)R_{B}; —CHO; —CO₂H; —CO₂R_{B}; —CN; —SCN; —SR_{B}; —SOR_{B}; —SO₂R_{B}; —NO₂; —N₃; —NH₂; —NHR_{B}; —N(R_{B})₂; —NHC(═O)R_{B}; —NR_{B}C(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₁ and R₂ may be taken together to form an epoxide ring, aziridine ring, cyclopropyl ring, or a bond of a carbon-carbon double bond;

R₃ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{C}; —C(═O)R_{C}; —CHO; —CO₂H; —CO₂R_{C}; —CN; —SCN; —SR_{C}; —SOR_{C}; —SO₂R_{C}; —NO₂; —N₃; —NH₂; —NHR_{C}; —N(R_{C})₂; —NHC(═O)R_{C}; —NR_{C}C(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₄ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{D}; —C(═O)R_{D}; —CHO; —CO₂H; —CO₂R_{D}; —CN; —SCN; —SR_{D}; —SOR_{D}; —SO₂R_{D}; —NO₂; —N₃; —NH₂; —NHR_{D}; —N(R_{D})₂; —NHC(═O)R_{D}; —NR_{D}C(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₅ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{E}; —C(═O)R_{E}; —CHO; —CO₂H; —CO₂R_{E}; —CN; —SCN; —SR_{E}; —SOR_{E}; —SO₂R_{E}; —NO₂; —N₃; —NH₂; —NHR_{E}; —N(R_{E})₂; —NHC(═O)R_{E}; —NR_{E}C(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₆ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{F}; —C(═O)R_{F}; —CHO; —CO₂H; —CO₂R_{F}; —CN; —SCN; —SR_{F}; —SOR_{F}; —SO₂R_{F}; —NO₂; —N₃; —NH₂; —NHR_{F}; —N(R_{F})₂; —NHC(═O)R_{F}; —NR_{F}C(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₇ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{G}; —C(═O)R_{G}; —CHO; —CO₂H; —CO₂R_{G}; —CN; —SCN; —SR_{G}; —SOR_{G}; —SO₂R_{G}; —NO₂; —N₃; —NH₂; —NHR_{G}; —N(R_{G})₂; —NHC(═O)R_{G}; —NR_{G}C(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₈ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{H}; —C(═O)R_{H}; —CHO; —CO₂H; —CO₂R_{H}; —CN; —SCN; —SR_{H}; —SOR_{H}; —SO₂R_{H}; —NO₂; —N₃; —NH₂; —NHR_{H}; —N(R_{H})₂; —NHC(═O)R_{H}; —NR_{H}C(═O)R_{H}; —NR_{H}C(═O)N(R_{H})₂; —OC(═O)OR_{H}; —OC(═O)R_{H}; —OC(═O)N(R_{H})₂; —NR_{H}C(═O)OR_{H}; or —C(R_{H})₃; wherein each occurrence of R_{H} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₉ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{I}; ═O; —C(═O)R_{I}; —CHO; —CO₂H; —CO₂R_{I}; —CN; —SCN; —SR_{I}; —SOR_{I}; —SO₂R_{I}; —NO₂; —N₃; —NH₂; —NHR_{I}; —N(R_{I})₂; —NHC(═O)R_{I}; —NR_{I}C(═O)R_{I}; —NR_{I}C(═O)N(R_{I})₂; —OC(═O)OR_{I}; —OC(═O)R_{I}; —OC(═O)N(R_{I})₂; —NR_{I}C(═O)OR_{I}; or —C(R_{I})₃; wherein each occurrence of R_{I} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₁₀ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{J}; ═O; —C(═O)R_{J}; —CHO; —CO₂H; —CO₂R_{J}; —CN; —SCN; —SR_{J}; —SOR_{J}; —SO₂R_{J}; —NO₂; —N₃; —NH₂; —NHR_{I}; —N(R_{J})₂; —NHC(═O)R_{J}; —NR_{J}C(═O)R_{J}; —NR_{J}C(═O)N(R_{J})₂; —OC(═O)OR_{J}; —OC(═O)R_{J}; —OC(═O)N(R)₂; —NR_{I}C(═O)OR_{J}; or —C(R_{J})₃; wherein each occurrence of R_{J} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety; and pharmaceutically acceptable salts, stereoisomers, tautomers, and pro-drugs thereof.

Celastrol, gedunin, and derivates thereof as described herein are useful in treating proliferative diseases. In certain embodiments, these compounds are useful in treating cancer. Any cancer that is susceptible to the inhibition of Hsp90 may be treated using the inventive compounds. In certain embodiments, the cancer being treated is dependent on Hsp90 for survival. In particular, the compounds described herein are useful in treating prostate cancer, breast cancer, leukemia, lymphoma, ovarian cancer, lung cancer, colon cancer, etc. The compounds are particularly useful in treating tumors driven by a mutated protein kinase or tumors driven by nuclear hormone receptors such as androgen receptor (prostate), estrogen receptor (breast), or progesterone receptor (breast). In certain embodiments, the cancer being treated is BCR/ABL chromic myeloid leukemia, an FLT3 mutant leukemia, an EGFR mutant lung cancer, or an AKT mutant cancer. The compounds may be used in combination with other cytotoxic agents or anti-neoplastic agents. In certain embodiments, the compound is combined with another Hsp90 inhibitor (e.g., 17-AAG). In certain other embodiments, the compounds are used to treat other proliferative disorders such as benign tumors, inflammatory diseases, and diabetic retinopathy. The compounds may also be used to treat infectious diseases (e.g., fungal infections). Methods of treatment, pharmaceutical compositions, and kits using the compounds described herein are also provided by the invention.

The inventive compounds are also useful as tools to probe biological function (e.g., the inhibition of Hsp90; the role of Hsp90 in the cell; the role of glucocorticoid receptors (e.g. androgen receptors) in th cell; the role of Hsp90 in stabilizing oncogenic proteins; the role of Hsp90 in stabilizing receptors; the effect of Hsp90 inhibition of glucocorticoid receptor activity). For example, the compounds may be administered to wild type cells or altered cells to understand the effect of Hsp90 in the cell. In certain embodiments, cancer cell lines are used.

### DEFINITIONS

Certain compounds of the present invention, and definitions of specific functional groups are also described in more detail below. For purposes of this invention, the chemical elements are identified in accordance with the Periodic Table of the Elements, CAS version, Handbook of Chemistry and Physics, 75^{th} Ed., inside cover, and specific functional groups are generally defined as described therein. Additionally, general principles of organic chemistry, as well as specific functional moieties and reactivity, are described in Organic Chemistry, Thomas Sorrell, University Science Books, Sausalito: 1999, the entire contents of which are incorporated herein by reference. Furthermore, it will be appreciated by one of ordinary skill in the art that the synthetic methods, as described herein, utilize a variety of protecting groups.

It will be appreciated that the compounds, as described herein, may be substituted with any number of substituents or functional moieties. In general, the term “substituted” whether preceded by the term “optionally” or not, and substituents contained in formulas of this invention, refer to the replacement of hydrogen radicals in a given structure with the radical of a specified substituent. When more than one position in any given structure may be substituted with more than one substituent selected from a specified group, the substituent may be either the same or different at every position. As used herein, the term “substituted” is contemplated to include all permissible substituents of organic compounds. In a broad aspect, the permissible substituents include acyclic and cyclic, branched and unbranched, carbocyclic and heterocyclic, aromatic and nonaromatic substituents of organic compounds. For purposes of this invention, heteroatoms such as nitrogen may have hydrogen substituents and/or any permissible substituents of organic compounds described herein which satisfy the valencies of the heteroatoms. Furthermore, this invention is not intended to be limited in any manner by the permissible substituents of organic compounds. Combinations of substituents and variables envisioned by this invention are preferably those that result in the formation of stable compounds useful in the treatment, for example of proliferative disorders, including, but not limited to cancer. The term “stable”, as used herein, preferably refers to compounds which possess stability sufficient to allow manufacture and which maintain the integrity of the compound for a sufficient period of time to be detected and preferably for a sufficient period of time to be useful for the purposes detailed herein.

The term “acyl”, as used herein, refers to a carbonyl-containing functionality, e.g., —C(═O)R′, wherein R′ is an aliphatic, alycyclic, heteroaliphatic, heterocyclic, aryl, heteroaryl, (aliphatic)aryl, (heteroaliphatic)aryl, heteroaliphatic(aryl) or heteroaliphatic(heteroaryl) moiety, whereby each of the aliphatic, heteroaliphatic, aryl, or heteroaryl moieties is substituted or unsubstituted, or is a substituted (e.g., hydrogen or aliphatic, heteroaliphatic, aryl, or heteroaryl moieties) oxygen or nitrogen containing functionality (e.g., forming a carboxylic acid, ester, or amide functionality).

The term “aliphatic”, as used herein, includes both saturated and unsaturated, straight chain (i.e., unbranched) or branched aliphatic hydrocarbons, which are optionally substituted with one or more functional groups. As will be appreciated by one of ordinary skill in the art, “aliphatic” is intended herein to include, but is not limited to, alkyl, alkenyl, alkynyl moieties. Thus, as used herein, the term “alkyl” includes straight and branched alkyl groups. An analogous convention applies to other generic terms such as “alkenyl”, “alkynyl” and the like. Furthermore, as used herein, the terms “alkyl”, “alkenyl”, “alkynyl” and the like encompass both substituted and unsubstituted groups. In certain embodiments, as used herein, “lower alkyl” is used to indicate those alkyl groups (substituted, unsubstituted, branched or unbranched) having 1-6 carbon atoms.

In certain embodiments, the alkyl, alkenyl and alkynyl groups employed in the invention contain 1-20 aliphatic carbon atoms. In certain other embodiments, the alkyl, alkenyl, and alkynyl groups employed in the invention contain 1-10 aliphatic carbon atoms. In yet other embodiments, the alkyl, alkenyl, and alkynyl groups employed in the invention contain 1-8 aliphatic carbon atoms. In still other embodiments, the alkyl, alkenyl, and alkynyl groups employed in the invention contain 1-6 aliphatic carbon atoms. In yet other embodiments, the alkyl, alkenyl, and alkynyl groups employed in the invention contain 1-4 carbon atoms. Illustrative aliphatic groups thus include, but are not limited to, for example, methyl, ethyl, n-propyl, isopropyl, allyl, n-butyl, sec-butyl, isobutyl, tert-butyl, n-pentyl, sec-pentyl, isopentyl, tert-pentyl, n-hexyl, sec-hexyl, moieties and the like, which again, may bear one or more substituents. Alkenyl groups include, but are not limited to, for example, ethenyl, propenyl, butenyl, 1-methyl-2-buten-1-yl, and the like. Representative alkynyl groups include, but are not limited to, ethynyl, 2-propynyl (propargyl), 1-propynyl and the like.

The term “alicyclic”, as used herein, refers to compounds which combine the properties of aliphatic and cyclic compounds and include but are not limited to cyclic, or polycyclic aliphatic hydrocarbons and bridged cycloalkyl compounds, which are optionally substituted with one or more functional groups. As will be appreciated by one of ordinary skill in the art, “alicyclic” is intended herein to include, but is not limited to, cycloalkyl, cycloalkenyl, and cycloalkynyl moieties, which are optionally substituted with one or more functional groups. Illustrative alicyclic groups thus include, but are not limited to, for example, cyclopropyl, —CH₂-cyclopropyl, cyclobutyl, —CH₂-cyclobutyl, cyclopentyl, —CH₂-cyclopentyl-n, cyclohexyl, —CH₂-cyclohexyl, cyclohexenylethyl, cyclohexanylethyl, norborbyl moieties and the like, which again, may bear one or more substituents.

The term “alkoxy” (or “alkyloxy”), or “thioalkyl” as used herein refers to an alkyl group, as previously defined, attached to the parent molecular moiety through an oxygen atom or through a sulfur atom. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkoxy, include but are not limited to, methoxy, ethoxy, propoxy, isopropoxy, n-butoxy, tert-butoxy, neopentoxy and n-hexoxy. Examples of thioalkyl include, but are not limited to, methylthio, ethylthio, propylthio, isopropylthio, n-butylthio, and the like.

The term “alkylamino” refers to a group having the structure —NHR′ wherein R′ is alkyl, as defined herein. The term “aminoalkyl” refers to a group having the structure NH₂R′—, wherein R′ is alkyl, as defined herein. In certain embodiments, the alkyl group contains 1-20 aliphatic carbon atoms. In certain other embodiments, the alkyl group contains 1-10 aliphatic carbon atoms. In yet other embodiments, the alkyl, alkenyl, and alkynyl groups employed in the invention contain 1-8 aliphatic carbon atoms. In still other embodiments, the alkyl group contains 1-6 aliphatic carbon atoms. In yet other embodiments, the alkyl group contains 1-4 aliphatic carbon atoms. Examples of alkylamino include, but are not limited to, methylamino, ethylamino, iso-propylamino and the like.

Some examples of substituents of the above-described aliphatic (and other) moieties of compounds of the invention include, but are not limited to aliphatic; heteroaliphatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alycyclic, heteroaliphatic, heterocyclic, aryl, heteroaryl, alkylaryl, or alkylheteroaryl, wherein any of the aliphatic, heteroaliphatic, alkylaryl, or alkylheteroaryl substituents described above and herein may be substituted or unsubstituted, branched or unbranched, cyclic or acyclic, and wherein any of the aryl or heteroaryl substituents described above and herein may be substituted or unsubstituted. Additional examples of generally applicable substituents are illustrated by the specific embodiments shown in the Examples that are described herein.

In general, the term “aryl”, as used herein, refers to a stable mono- or polycyclic, unsaturated moiety having preferably 3-14 carbon atoms, each of which may be substituted or unsubstituted. In certain embodiments, the term “aryl” refers to a planar ring having p-orbitals perpendicular to the plane of the ring at each ring atom and satisfying the Huckel rule where the number of pi electrons in the ring is (4n+2) wherein n is an integer. A mono- or polycyclic, unsaturated moiety that does not satisfy one or all of these criteria for aromaticity is defined herein as “non-aromatic”, and is encompassed by the term “alicyclic”.

In general, the term “heteroaryl”, as used herein, refers to a stable mono- or polycyclic, unsaturated moiety having preferably 3-14 carbon atoms, each of which may be substituted or unsubstituted; and comprising at least one heteroatom selected from O, S and N within the ring (i.e., in place of a ring carbon atom). In certain embodiments, the term “heteroaryl” refers to a planar ring comprising at least on heteroatom, having p-orbitals perpendicular to the plane of the ring at each ring atom, and satisfying the Huckel rule where the number of pi electrons in the ring is (4n+2) wherein n is an integer.

It will also be appreciated that aryl and heteroaryl moieties, as defined herein may be attached via an alkyl or heteroalkyl moiety and thus also include -(alkyl)aryl, -(heteroalkyl)aryl, -(heteroalkyl)heteroaryl, and -(heteroalkyl)heteroaryl moieties. Thus, as used herein, the phrases “aryl or heteroaryl moieties” and “aryl, heteroaryl, -(alkyl)aryl, -(heteroalkyl)aryl,-(heteroalkyl)heteroaryl, and -(heteroalkyl)heteroaryl” are interchangeable. Substituents include, but are not limited to, any of the previously mentioned substituents, i.e., the substituents recited for aliphatic moieties, or for other moieties as disclosed herein, resulting in the formation of a stable compound.

The term “aryl”, as used herein, does not differ significantly from the common meaning of the term in the art, and refers to an unsaturated cyclic moiety comprising at least one aromatic ring. In certain embodiments, “aryl” refers to a mono- or bicyclic carbocyclic ring system having one or two aromatic rings including, but not limited to, phenyl, naphthyl, tetrahydronaphthyl, indanyl, indenyl and the like.

The term “heteroaryl”, as used herein, does not differ significantly from the common meaning of the term in the art, and refers to a cyclic aromatic radical having from five to ten ring atoms of which one ring atom is selected from S, O and N; zero, one or two ring atoms are additional heteroatoms independently selected from S, O and N; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

It will be appreciated that aryl and heteroaryl groups (including bicyclic aryl groups) can be unsubstituted or substituted, wherein substitution includes replacement of one or more of the hydrogen atoms thereon independently with any one or more of the following moieties including, but not limited to: aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)Rₓ; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl, heteroaryl, -(alkyl)aryl or -(alkyl)heteroaryl substituents described above and herein may be substituted or unsubstituted. Additionally, it will be appreciated, that any two adjacent groups taken together may represent a 4, 5, 6, or 7-membered substituted or unsubstituted alicyclic or heterocyclic moiety. Additional examples of generally applicable substituents are illustrated by the specific embodiments shown in the Examples that are described herein.

The term “cycloalkyl”, as used herein, refers specifically to groups having three to seven, preferably three to ten carbon atoms. Suitable cycloalkyls include, but are not limited to cyclopropyl, cyclobutyl, cyclopentyl, cyclohexyl, cycloheptyl and the like, which, as in the case of aliphatic, alicyclic, heteroaliphatic or heterocyclic moieties, may optionally be substituted with substituents including, but not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heteroaliphatic”, as used herein, refers to aliphatic moieties in which one or more carbon atoms in the main chain have been substituted with a heteroatom. Thus, a heteroaliphatic group refers to an aliphatic chain which contains one or more oxygen, sulfur, nitrogen, phosphorus or silicon atoms, e.g., in place of carbon atoms. Heteroaliphatic moieties may be linear or branched, and saturated or unsaturated. In certain embodiments, heteroaliphatic moieties are substituted by independent replacement of one or more of the hydrogen atoms thereon with one or more moieties including, but not limited to aliphatic; alicyclic; heteroaliphatic; heterocyclic; aromatic; heteroaromatic; aryl; heteroaryl; alkylaryl; alkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substituents described above and herein may be substituted or unsubstituted. Additional examples of generally applicable substituents are illustrated by the specific embodiments shown in the Examples that are described herein.

The term “heterocycloalkyl”, “heterocycle” or “heterocyclic”, as used herein, refers to compounds which combine the properties of heteroaliphatic and cyclic compounds and include, but are not limited to, saturated and unsaturated mono- or polycyclic cyclic ring systems having 5-16 atoms wherein at least one ring atom is a heteroatom selected from O, S and N (wherein the nitrogen and sulfur heteroatoms may be optionally be oxidized), wherein the ring systems are optionally substituted with one or more functional groups, as defined herein. In certain embodiments, the term “heterocycloalkyl”, “heterocycle” or “heterocyclic” refers to a non-aromatic 5-, 6- or 7-membered ring or a polycyclic group wherein at least one ring atom is a heteroatom selected from O, S and N (wherein the nitrogen and sulfur heteroatoms may be optionally be oxidized), including, but not limited to, a bi- or tri-cyclic group, comprising fused six-membered rings having between one and three heteroatoms independently selected from oxygen, sulfur and nitrogen, wherein (i) each 5-membered ring has 0 to 2 double bonds, each 6-membered ring has 0 to 2 double bonds and each 7-membered ring has 0 to 3 double bonds, (ii) the nitrogen and sulfur heteroatoms may be optionally be oxidized, (iii) the nitrogen heteroatom may optionally be quaternized, and (iv) any of the above heterocyclic rings may be fused to an aryl or heteroaryl ring. Representative heterocycles include, but are not limited to, heterocycles such as furanyl, thiofuranyl, pyranyl, pyrrolyl, thienyl, pyrrolidinyl, pyrazolinyl, pyrazolidinyl, imidazolinyl, imidazolidinyl, piperidinyl, piperazinyl, oxazolyl, oxazolidinyl, isooxazolyl, isoxazolidinyl, dioxazolyl, thiadiazolyl, oxadiazolyl, tetrazolyl, triazolyl, thiatriazolyl, oxatriazolyl, thiadiazolyl, oxadiazolyl, morpholinyl, thiazolyl, thiazolidinyl, isothiazolyl, isothiazolidinyl, dithiazolyl, dithiazolidinyl, tetrahydrofuryl, and benzofused derivatives thereof. In certain embodiments, a “substituted heterocycle, or heterocycloalkyl or heterocyclic” group is utilized and as used herein, refers to a heterocycle, or heterocycloalkyl or heterocyclic group, as defined above, substituted by the independent replacement of one, two or three of the hydrogen atoms thereon with but are not limited to aliphatic; alicyclic; heteroaliphatic; heterocyclic; aromatic; heteroaromatic; aryl; heteroaryl; alkylaryl; heteroalkylaryl; alkylheteroaryl; heteroalkylheteroaryl; alkoxy; aryloxy; heteroalkoxy; heteroaryloxy; alkylthio; arylthio; heteroalkylthio; heteroarylthio; F; Cl; Br; I; —OH; —NO₂; —CN; —CF₃; —CH₂CF₃; —CHCl₂; —CH₂OH; —CH₂CH₂OH; —CH₂NH₂; —CH₂SO₂CH₃; —C(O)Rₓ; —CO₂(Rₓ); —CON(Rₓ)₂; —OC(O)Rₓ; —OCO₂Rₓ; —OCON(Rₓ)₂; —N(Rₓ)₂; —S(O)₂Rₓ; —NRₓ(CO)Rₓ wherein each occurrence of Rₓ independently includes, but is not limited to, aliphatic, alicyclic, heteroaliphatic, heterocyclic, aromatic, heteroaromatic, aryl, heteroaryl, alkylaryl, alkylheteroaryl, heteroalkylaryl or heteroalkylheteroaryl, wherein any of the aliphatic, alicyclic, heteroaliphatic, heterocyclic, alkylaryl, or alkylheteroaryl substituents described above and herein may be substituted or unsubstituted, branched or unbranched, saturated or unsaturated, and wherein any of the aromatic, heteroaromatic, aryl or heteroaryl substitutents described above and herein may be substituted or unsubstituted. Additional examples or generally applicable substituents are illustrated by the specific embodiments shown in the Examples, which are described herein.

Additionally, it will be appreciated that any of the alicyclic or heterocyclic moieties described above and herein may comprise an aryl or heteroaryl moiety fused thereto. Additional examples of generally applicable substituents are illustrated by the specific embodiments shown in the Examples that are described herein. The terms “halo” and “halogen” as used herein refer to an atom selected from fluorine, chlorine, bromine and iodine.

The terms “halo” and “halogen” as used herein refer to an atom selected from fluorine, chlorine, bromine and iodine.

The term “haloalkyl” denotes an alkyl group, as defined above, having one, two, or three halogen atoms attached thereto and is exemplified by such groups as chloromethyl, bromoethyl, trifluoromethyl, and the like.

The term “amino”, as used herein, refers to a primary (—NH₂), secondary (—NHRₓ), tertiary (—NRₓR_{y}) or quaternary (—N⁺RₓR_{y}R_{z}) amine, where Rₓ, R_{y} and R_{z} are independently an aliphatic, alicyclic, heteroaliphatic, heterocyclic, aryl, or heteroaryl moiety, as defined herein. Examples of amino groups include, but are not limited to, methylamino, dimethylamino, ethylamino, diethylamino, diethylaminocarbonyl, methylethylamino, iso-propylamino, piperidino, trimethylamino, and propylamino.

Unless otherwise indicated, as used herein, the terms “alkyl”, “alkenyl”, “alkynyl”, “heteroalkyl”, “heteroalkenyl”, “heteroalkynyl”, “alkylidene”, alkenylidene”, -(alkyl)aryl, -(heteroalkyl)aryl, -(heteroalkyl)aryl, -(heteroalkyl)heteroaryl, and the like encompass substituted and unsubstituted, and linear and branched groups. Similarly, the terms “aliphatic”, “heteroaliphatic”, and the like encompass substituted and unsubstituted, saturated and unsaturated, and linear and branched groups. Similarly, the terms “cycloalkyl”, “heterocycle”, “heterocyclic”, and the like encompass substituted and unsubstituted, and saturated and unsaturated groups. Additionally, the terms “cycloalkenyl”, “cycloalkynyl”, “heterocycloalkenyl”, “heterocycloalkynyl”, “aromatic”, “heteroaromatic, “aryl”, “heteroaryl” and the like encompass both substituted and unsubstituted groups.

The phrase, “pharmaceutically acceptable derivative”, as used herein, denotes any pharmaceutically acceptable salt, ester, or salt of such ester, of such compound, or any other adduct or derivative which, upon administration to a patient, is capable of providing (directly or indirectly) a compound as otherwise described herein, or a metabolite or residue thereof. Pharmaceutically acceptable derivatives thus include among others pro-drugs. A pro-drug is a derivative of a compound, usually with significantly reduced pharmacological activity, which contains an additional moiety, which is susceptible to removal in vivo yielding the parent molecule as the pharmacologically active species. An example of a pro-drug is an ester, which is cleaved in vivo to yield a compound of interest. Pro-drugs of a variety of compounds, and materials and methods for derivatizing the parent compounds to create the pro-drugs, are known and may be adapted to the present invention. Pharmaceutically acceptable derivatives also include “reverse pro-drugs.” Reverse pro-drugs, rather than being activated, are inactivated upon absorption. For example, as discussed herein, many of the ester-containing compounds of the invention are biologically active but are inactivated upon exposure to certain physiological environments such as a blood, lymph, serum, extracellular fluid, etc. which contain esterase activity. The biological activity of reverse pro-drugs and pro-drugs may also be altered by appending a functionality onto the compound, which may be catalyzed by an enzyme. Also, included are oxidation and reduction reactions, including enzyme-catalyzed oxidation and reduction reactions. Certain exemplary pharmaceutical compositions and pharmaceutically acceptable derivatives will be discussed in more detail herein below.

By the term “protecting group”, has used herein, it is meant that a particular functional moiety, e.g., O, S, or N, is temporarily blocked so that a reaction can be carried out selectively at another reactive site in a multifunctional compound. In preferred embodiments, a protecting group reacts selectively in good yield to give a protected substrate that is stable to the projected reactions; the protecting group must be selectively removed in good yield by readily available, preferably nontoxic reagents that do not attack the other functional groups; the protecting group forms an easily separable derivative (more preferably without the generation of new stereogenic centers); and the protecting group has a minimum of additional functionality to avoid further sites of reaction. As detailed herein, oxygen, sulfur, nitrogen and carbon protecting groups may be utilized. For example, in certain embodiments, as detailed herein, certain exemplary oxygen protecting groups are utilized. These oxygen protecting groups include, but are not limited to methyl ethers, substituted methyl ethers (e.g., MOM (methoxymethyl ether), MTM (methylthiomethyl ether), BOM (benzyloxymethyl ether), PMBM or MPM (p-methoxybenzyloxymethyl ether), to name a few), substituted ethyl ethers, substituted benzyl ethers, silyl ethers (e.g., TMS (trimethylsilyl ether), TES (triethylsilylether), TIPS (triisopropylsilyl ether), TBDMS (t-butyldimethylsilyl ether), tribenzyl silyl ether, TBDPS (t-butyldiphenyl silyl ether), to name a few), esters (e.g., formate, acetate, benzoate (Bz), trifluoroacetate, dichloroacetate, to name a few), carbonates, cyclic acetals and ketals. In certain other exemplary embodiments, nitrogen protecting groups are utilized. These nitrogen protecting groups include, but are not limited to, carbamates (including methyl, ethyl and substituted ethyl carbamates (e.g., Troc), to name a few) amides, cyclic imide derivatives, N-Alkyl and N-Aryl amines, imine derivatives, and enamine derivatives, to name a few. Certain other exemplary protecting groups are detailed herein, however, it will be appreciated that the present invention is not intended to be limited to these protecting groups; rather, a variety of additional equivalent protecting groups can be readily identified using the above criteria and utilized in the present invention. Additionally, a variety of protecting groups are described in Protective Groups in Organic Synthesis, Third Ed. Greene, T. W. and Wuts, P. G., Eds., John Wiley & Sons, New York: 1999, the entire contents of which are hereby incorporated by reference.

“Compound”: The term “compound” or “chemical compound” as used herein can include organometallic compounds, organic compounds, metals, transitional metal complexes, and small molecules. In certain preferred embodiments, polynucleotides are excluded from the definition of compounds. In other preferred embodiments, polynucleotides and peptides are excluded from the definition of compounds. In a particularly preferred embodiment, the term compounds refers to small molecules (e.g., preferably, non-peptidic and non-oligomeric) and excludes peptides, polynucleotides, transition metal complexes, metals, and organometallic compounds.

“Small Molecule”: As used herein, the term “small molecule” refers to a non-peptidic, non-oligomeric organic compound either synthesized in the laboratory or found in nature. Small molecules, as used herein, can refer to compounds that are “natural product-like”, however, the term “small molecule” is not limited to “natural product-like” compounds. Rather, a small molecule is typically characterized in that it contains several carbon-carbon bonds, and has a molecular weight of less than 1500, although this characterization is not intended to be limiting for the purposes of the present invention. Examples of “small molecules” that occur in nature include, but are not limited to, taxol, dynemicin, and rapamycin. In certain other preferred embodiments, natural-product-like small molecules are utilized.

“Natural Product-Like Compound”: As used herein, the term “natural product-like compound” refers to compounds that are similar to complex natural products which nature has selected through evolution. Typically, these compounds contain one or more stereocenters, a high density and diversity of functionality, and a diverse selection of atoms within one structure. In this context, diversity of functionality can be defined as varying the topology, charge, size, hydrophilicity, hydrophobicity, and reactivity to name a few, of the functional groups present in the compounds. The term, “high density of functionality”, as used herein, can preferably be used to define any molecule that contains preferably three or more latent or active diversifiable functional moieties. These structural characteristics may additionally render the inventive compounds functionally reminiscent of complex natural products, in that they may interact specifically with a particular biological receptor, and thus may also be functionally natural product-like.

As used herein the term “biological sample” includes, without limitation, cell cultures or extracts thereof; biopsied material obtained from an animal (e.g., mammal) or extracts thereof; and blood, saliva, urine, feces, semen, tears, or other body fluids or extracts thereof. For example, the term “biological sample” refers to any solid or fluid sample obtained from, excreted by or secreted by any living organism, including single-celled micro-organisms (such as bacteria and yeasts) and multicellular organisms (such as plants and animals, for instance a vertebrate or a mammal, and in particular a healthy or apparently healthy human subject or a human patient affected by a condition or disease to be diagnosed or investigated). The biological sample can be in any form, including a solid material such as a tissue, cells, a cell pellet, a cell extract, cell homogenates, or cell fractions; or a biopsy, or a biological fluid. The biological fluid may be obtained from any site (e.g. blood, saliva (or a mouth wash containing buccal cells), tears, plasma, serum, urine, bile, cerebrospinal fluid, amniotic fluid, peritoneal fluid, and pleural fluid, or cells therefrom, aqueous or vitreous humor, or any bodily secretion), a transudate, an exudate (e.g. fluid obtained from an abscess or any other site of infection or inflammation), or fluid obtained from a joint (e.g. a normal joint or a joint affected by disease such as rheumatoid arthritis, osteoarthritis, gout or septic arthritis). The biological sample can be obtained from any organ or tissue (including a biopsy or autopsy specimen) or may comprise cells (whether primary cells or cultured cells) or medium conditioned by any cell, tissue or organ. Biological samples may also include sections of tissues such as frozen sections taken for histological purposes. Biological samples also include mixtures of biological molecules including proteins, lipids, carbohydrates and nucleic acids generated by partial or complete fractionation of cell or tissue homogenates. Although the sample is preferably taken from a human subject, biological samples may be from any animal, plant, bacteria, virus, yeast, etc. The term animal, as used herein, refers to humans as well as non-human animals, at any stage of development, including, for example, mammals, birds, reptiles, amphibians, fish, worms and single cells. Cell cultures and live tissue samples are considered to be pluralities of animals. In certain exemplary embodiments, the non-human animal is a mammal (e.g., a rodent, a mouse, a rat, a rabbit, a monkey, a dog, a cat, a sheep, cattle, a primate, or a pig). An animal may be a transgenic animal or a human clone. If desired, the biological sample may be subjected to preliminary processing, including preliminary separation techniques.

### BRIEF DESCRIPTION OF THE DRAWING

FIG. 1 shows the structures of various Hsp90 inhibitors including celastrol, pristimerol, dihydrocelastrol, gedunin, deoxygedunin, deacetylgedunin, geldanamycin, and 17-allylamino-geldanamycin (17-AAG).

FIG. 2 shows a comparison of gene expression signatures for known Hsp90 inhibitors in MCF7 and LNCaP cells as compared to celastrol. Celastrol has been found to give a gene expression signature similar to Hsp90 inhibition by existing inhibitors in MCF7 and LNCaP cells. FIG. 2A shows the signature of celastrol treatment (1 μM, 6 hours) in MCF7 cells, which is similar to those signatures of geldanamycin and 17-AAG treatment by modified Komologrov-Smirnoff test, as indicated by the similarity rank out of 558 diverse compound treatments and enrichments score. FIG. 2B shows that the signature of celastrol treatment (1 μM, 6 hours) in LNCaP cells is also similar to those of geldanamycin and 17-AAG treatment in MCF7 cells by modified Komologrov-Smirnoff test.

FIG. 3 demonstrates that celastrol treatment decreases the levels of Hsp90 client proteins. FIG. 3A shows that celastrol treatment (1.25 μM, 24 hours) decreases the levels of androgen receptor (AR), epidermal growth factor receptor (EGFR), Raf-1, and FLT3 in the LNCaP prostate cancer cell line. FIG. 3B demonstrates that celastrol and gedunin treatment (24 hours) decreases bcr-abl levels in K562 cells. 17-AAG treatment is shown as a control.

FIG. 4 shows that celastrol represses androgen receptor signaling. FIG. 4A demonstrates that celastrol treatment reverts a selected androgen signaling signature to an androgen deprived signature in LNCaP cells in a concentration dependent manner. A heat map shows the relative expression of genes in the androgen signaling signature under androgen deprivation and androgen stimulation alone, with AR inhibitor treatment, and with celastrol treatment. FIG. 4B shows that gedunin treatment reverts a selected androgen signaling signature to an androgen deprived signature in LNCaP cells in a concentration dependent manner. FIG. 4C demonstrates that celastrol treatment suppresses a broader androgen signaling signature determined by genome-wide microarray analysis. Upon celastrol treatment, androgen-responsive gene expression of celastrol-treated, androgen-stimulated cells cluster with that of androgen deprived cells, rather than that of androgen cells, by hierarchical clustering.

FIG. 5 shows the identification of inhibitors of androgen signaling signature by a gene expression-based screen. A: A high-throughput method for quantifying transcript levels was developed to enable gene expression signature-based screens. In this method, mRNA in cell lysates is hybridized to dT20-conjugated plates and then reverse transcribed. The resulting covalently attached cDNA is amplified by ligation-mediated PCR. For each gene to be assayed, ligation generates a sequence complementary to the transcript and flanked by a unique barcode tag and universal primer sites. The ligation product is PCR amplified using biotin-conjugated universal primers. The PCR products are then captured by hybridization to probes complementary to the barcodes that are attached to uniquely colored polystyrene beads. The products are subsequently stained with streptavidin-phycoerythrin (SAPE). Each gene product is identified by the color of its capture bead and quantified using the associated SAPE fluorescence, as measured by two-laser flow cytometry. B: A gene expression signature of androgen stimulation was defined from gene expression profiles of LNCaP cells stimulated with the synthetic androgen R1881 for 12 hr and 24 hr, as compared to androgen-deprived LNCaP cells. The 27 gene signature contains both androgen-induced and androgen-repressed genes, shown here by row-normalized heat map. C: GE-HTS screen identifies a family of celastrol and gedunin compounds that revert the androgen signaling signature to the androgen-deprived signature in LNCaP cells. LNCaP cells were treated with 1 nM R1881 plus compounds at ˜20 μM for 24 hr. The heat map shows the row-normalized signatures induced by gedunin and celastrol compounds in the screen and the competitive AR inhibitor casodex (bicalutamide).

FIG. 6 shows the inhibition of androgen signaling by celastrol and gedunin. A: Structures of celastrol and gedunin are shown (top). Derivatives of celastrol (left, bottom) and gedunin (right, bottom) identified as AR signature inhibitors by GE-HTS are also shown. B: Celastrol and gedunin inhibit the GE-HTS androgen signaling signature in a concentration-dependent manner. LNCaP cells were treated with 1 nM R1881 for 12 hr and then 1 nM R1881 plus compound for an additional 24 hr. Controls were treated with vehicle in place of R1881 and/or compound. The row-normalized GE-HTS signature shows concentration-dependent reversion to the androgen deprivation signature. C: Celastrol- and gedunin-mediated effects on androgen-responsive gene expression mimics androgen deprivation. Average link hierarchical clustering was carried out on androgen-responsive gene expression from androgen-deprived cells (green) and androgen-treated cells with vehicle (red), celastrol (1.25 μM, 24 hr, blue), or gedunin (20 μM, 24 hr, yellow). The dendrograms show the clustering of the samples within the androgen-responsive gene space. D: Celastrol and gedunin inhibit anchorage-independent prostate cancer cell growth. Celastrol and gedunin inhibit LNCaP colony formation in soft agar (mean of three replicates ±1 SD). E: Celastrol and gedunin inhibit adherent prostate cancer cell growth. Celastrol (red) and gedunin (black) inhibit growth of LNCaP cells, as determined by luminescent assay of ATP level (mean of four replicates ±1 SD).

FIG. 7 demonstrates that the gene expression compendium of drug treatment predicts HSP90-inhibitory activity of celastrol and gedunin and that celastrol and gedunin do inhibit the HSP90 pathway. A: Celastrol and gedunin gene expression signatures are similar to the gene expression profiles of HSP90 inhibition. From a collection of gene expression profiles representing 164 compounds, the expression profiles of 17-AAG, 17-DMAG, and geldanamycin treatment (6 hr, MCF7) show enrichment of celastrol (1.25 μM, 6 hr, LNCaP) and gedunin (20 μM, 6 hr, LNCaP cells) signatures at 6 hr. The barview is constructed from 453 horizontal lines, each representing an individual treatment instance and ordered by their corresponding enrichment with the celastrol and gedunin query signatures. All geldanamycin (n=6), 17-allylamino-geldanamycin (n=18), and 17-dimethylamino-geldanamycin (n=2) instances are colored in black. Colors applied to the remaining instances reflect positive (green), negative (red), or no (gray) enrichment with the celastrol and gedunin query signatures. The combined barview is constructed from horizontal lines, each representing a compound treatment and ordered as for the single instance barview. B: Enrichment of the celastrol and gedunin signatures in a selected 17-AAG instance. Celastrol and gedunin induce (green) and repress (red) gene probes that are enriched in the 17-AAG gene expression profile (22,283 probe sets), ordered by their extent of differential expression between treatment and control scans for the 17-AAG instance (x axis). The Kolmogorov-Smirnov score is shown for the induced and repressed signatures of celastrol (1.25 μM, 6 hr, LNCaP) and gedunin (20 μM, 6 hr, LNCaP) across the best matched 17-AAG gene expression profile (1 μM, 6 hr, MCF7). The fact that most celastrol- and gedunin-induced genes appear early in the ordered 17-AAG profile and are therefore enriched in the 17-AAG-induced signature is illustrated by this graphical representation of the Kolmogorov-Smirnov analysis (green). The converse is true for the repressed genes (red). A signature populated with randomly selected probe sets shows no enrichment. C: Celastrol and gedunin lower HSP90 client protein levels. Celastrol and gedunin induce concentration-dependent decreases in AR level at 24 hr. 17-AAG treatment is shown as a positive control. D: Celastrol and gedunin decrease the levels of HSP90 clients BCR-ABL1, EGFR, and FLT3 Celastrol and gedunin treatment for 24 hr lowers EGFR levels in LNCaP cells, BCR-ABL1 levels, and phosphorylation in K562 cells, and FLT3, EGFR, and BCR-ABL1 levels in Ba/F3 cells. Ba/F3 cells overexpressing BCR-ABL1 were particularly susceptible to death upon celastrol treatment, resulting in lowered total protein level at 7.5 μM celastrol. E: Cellular treatment with celastrol and gedunin inhibits HSP90 ATP-binding activity. HSP90 from lysates of celastrol- or gedunin-treated LNCaP and K562 cells show decreased binding to ATP-polystyrene relative to vehicle-treated cells. ATP-binding proteins were isolated from treated LNCaP and K562 cells by ATP affinity purification and detected by western blot. Affinity-purified proteins (pulldown) and total lysate were blotted for HSP90α, control ATP-binding proteins CSK (LNCaP), DDR1 (K562), and actin. F: Celastrol decreases HSP90 interaction with its cochaperone p23. Celastrol treatment of SKBR-3 cells (2.5 μM, 12 hr) decreased the amount of p23 that coimmunoprecipitated with HSP90, as shown by western blot of the coimmunoprecipitate and lysate. Celastrol did not affect the amount of coimmunoprecipitating HOP, shown as a control. The C-terminal HSP90 inhibitor PU24FCI (20 μM, 24 hr) is shown as a control.

FIG. 8 shows that celastrol and gedunin inhibit HSP90 function through a different mechanism than existing HSP90 ATP-binding pocket inhibitors. A: Celastrol (black squares) and gedunin (black upward triangles) do not compete with Cy3B-labeled geldanamycin for binding to the ATP-binding site of HSP90α in vitro at pharmacological doses, unlike N-terminal inhibitors 17-AAG (black downward triangles) and PU-H71 (black diamonds). The decrease in fluorescence polarization of Cy3B-geldanamycin upon displacement from the ATP-binding pocket of recombinant hHSP90α is shown. The novobiocin-analog coumermycin A (white squares) is shown as a C-terminal binding control. The mean±1 SD is shown. B: Celastrol and gedunin show synergistic inhibition of AR signaling with the HSP90 inhibitor 17-AAG. The combined effect of these compounds and 17-AAG on the LNCaP androgen signaling signature at 24 hr is shown by isobologram. Synergy appears as points below the line of additivity. C: Celastrol and gedunin show synergistic growth inhibition with 17-AAG. The combined effect of these compounds and 17-AAG on LNCaP cell viability at 24 hr, as determined by ATP level, is shown by isobologram.

FIG. 9 shows the structures of strong hits identified by a gene expression-based screen for androgen signaling inhibitors. Compounds shown scored as inducers of an androgen signaling deprivation signature in at least two of three replicates with P<0.05 by three metrics, weighted summed expression, K nearest neighbors, and Naïve Bayes classifier. A: Celastrol and gedunin derivatives that scored as strong hits. B: Structures of all other strong hits, including several steroid compounds.

FIG. 10 shows the structures of weak hits identified by a gene expression-based screen for androgen signaling inhibitors. Compounds shown scored as inducers of an androgen signaling deprivation signature in at least two of three replicates with P<0.05 by two of three metrics. A: Celastrol and gedunin derivatives that scored as weak hits. B: Steroids formed a major class of weak androgen signalling signature inhibitors, shown here. These include estrogens, glucocorticoids, and progesterones. C: All other weak hits are shown here.

FIG. 11 demonstrate that celastrol and gedunin show synergistic growth inhibition with geldanamycin. The combined effect of celastrol or gedunin with geldanamycin on AR signaling and cell viability, as determined by ATP level, in LNCaP cells is shown by isobologram.

FIG. 12 demonstrates that gedunin modulates the HSP90 pathway. A: Chemical structure of gedunin and 17-allylamino-geldanamycin. B: Gedunin is connected with geldanamycin and its analogs. Barview showing all 17-allylamino-geldanamycin (n=18), geldanamycin (n=6), and 17-dimethylamino-geldanamycin (n=2) instances for the gedunin signature. C: Gedunin lowers the levels of HSP90-interacting proteins, including the androgen receptor (AR), in LNCaP cells and Ba/F3 cells ectopically expressing them. Mutant HSP90-interacting proteins (BCR-ABL T315I point mutant and the FLT3-ITD internal tandem duplication mutant) show increased sensitivity to gedunin treatment.

FIG. 13 shows that celastrol acts through a different mechanism than existing HSP90 inhibitors. A: Celastrol does not compete with Cy3-labeled geldanamycin for binding to the ATP binding site of HSP90α in vitro (blue line), unlike 17-AAG (black line). The decrease in fluorescence polarization of Cy3-geldanamycin upon displacement from the ATP binding pocket by 17-AAG is shown as a control. C: Celastrol shows synergy with HSP90 inhibitors. The combined effect of celastrol and HSP90 inhibitors on the androgen signaling signature is determined by the Bliss equation and depicted by heat map. Synergy, as defined by the Bliss score, appears in red, and antagonism appears in blue.

FIG. 14 shows the inhibition of cancer cell growth by celastrol and gedunin. Celastrol and gedunin inhibit androgen-sensitive prostate cancer cell growth, as assayed by ATP level. Growth curves of vehicle-treated androgen-stimulated (heavy black) and androgen-deprived (blue) are shown as controls.

FIG. 15 shows that celastrol inhibits the conformational change of HSP90 induced by 1,1′-bis(4-anilino-5-naphthalenesulfonic acid (bis-ANS). The effect of celastrol is seen at moderate concentrations. The effect of gedunin is not clearly observed possibly due to the relatively high concentrations needed for gedunin's effect.

FIG. 16 shows various exemplary reactions useful in preparing celastrol analogs.

FIG. 17 shows various exemplary reactions useful in preparing gedunin analogs.

### DETAILED DESCRIPTION OF THE INVENTION

Celastrol, gedunin, and derivative thereof as described herein have been found to be inhibitors of Hsp90. These compounds are therefore useful in the treatment of conditions in which Hsp90 inhibition is attractive. For example, other Hsp90 inhibitors have been found to be useful in the treatment of cancer. Hsp90 inhibitors are also useful in the treatment of other disease including fungal infections. Without wishing to be bound by any particular theory, it is thought that the activity of Hsp90 is necessary for stabilizing such important cellular proteins as receptors, transcription factors, kinases, and oncogenic proteins. Therefore, the inhibition of Hsp90 activity will destabilize these important cell proteins and lead to cell death.

Celastrol and gedunin were found to function as Hsp90 inhibitors in a screen of a small molecule library for compounds with the ability to modulate a gene expression signature indicative of androgen receptor (AR) activation in prostate cancer cells. Approximately 2,500 compounds were screened using LNCaP prostate cancer cells treated with androgen and a Luminex bead-based profiling method to measure the gene expression signature of AR activity following treatment. Peck et al., “A Method for High-Throughput Gene Expression Signature Analysis” Genome Biology, submitted Mar. 21, 2006; incorporated herein by reference. Several hits were identified in the screen including celastrol, celastrol derivatives, gedunin, and gedunin derivatives. To determine the mechanism of action of these identified compounds, the gene expression signature of celastrol treatment was compared to a database of gene expression signatures based on drug action. Pattern matching was observed for a number of drugs in the database. In particular, the known heat shock proteins Hsp90 inhibitors, geldanamycin and 17-AAG, were found to exhibit a similar gene expression signature. Therefore, celastrol, though structurally distinct, was found to functions as an Hsp90 inhibitor even though this activity of celatrol and the other identified compounds was previously unknown.

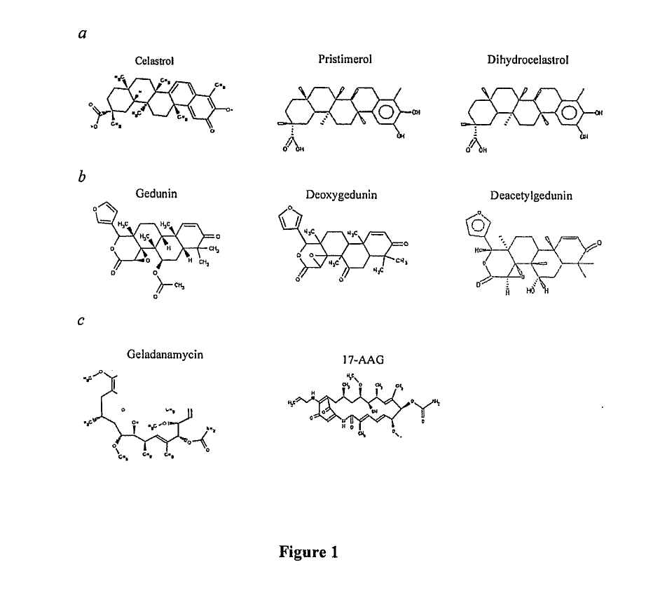

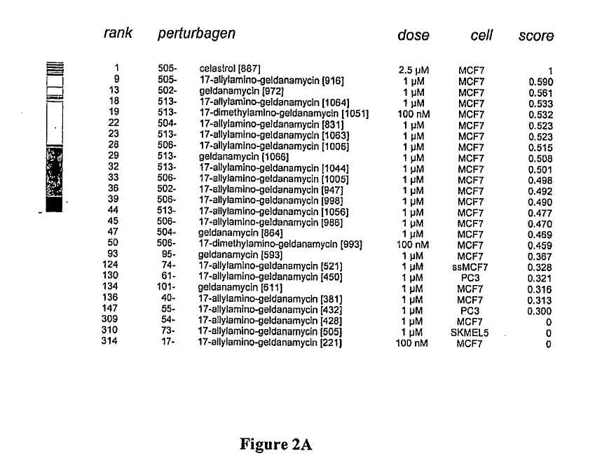

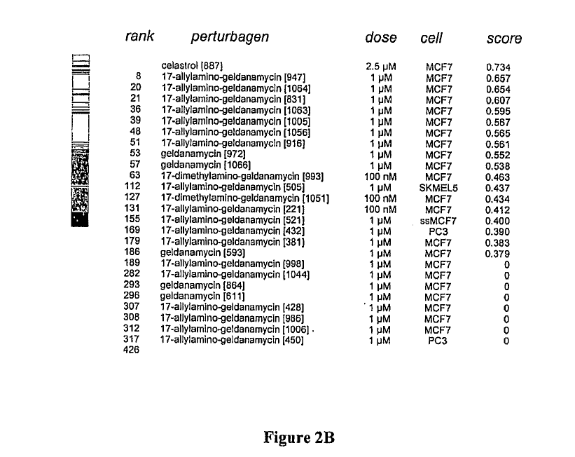

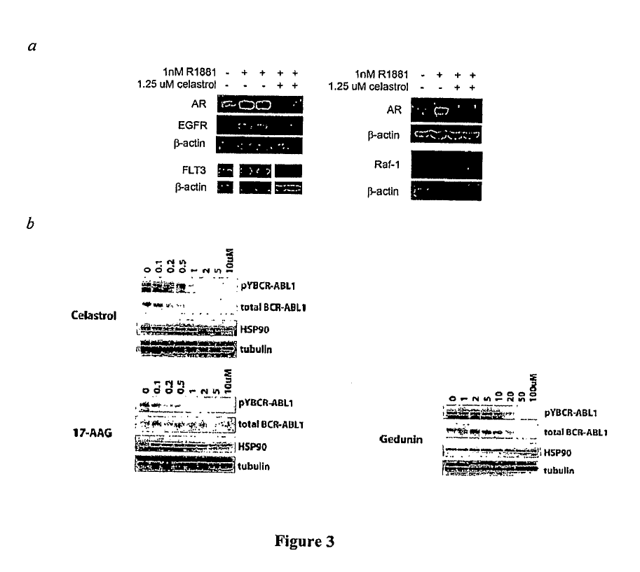

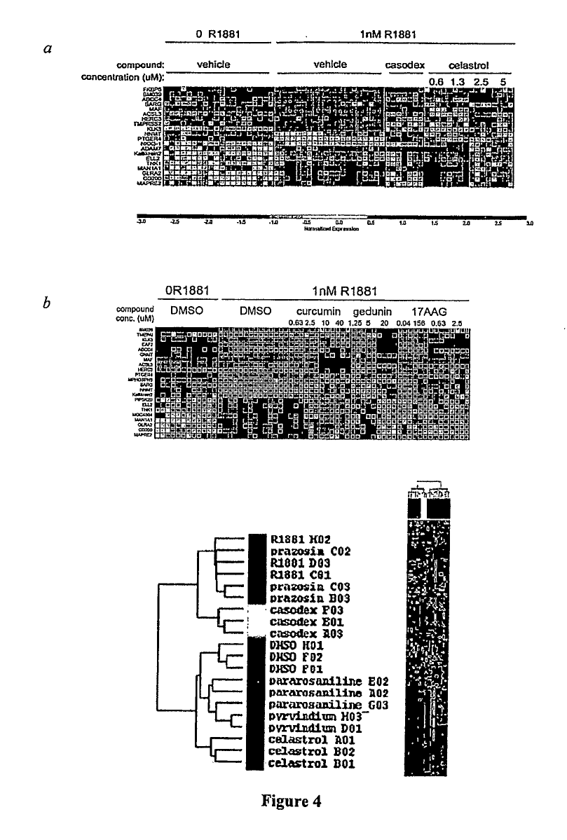

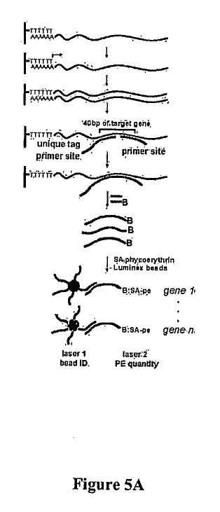

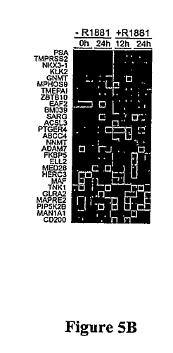

Celastrol treatment of cancer cell lines invokes a gene expression signature similar to that of Hsp90 inhibition by existing inhibitors (FIG. 2). Treatment of cancer cell lines with celastrol or gedunin significantly decreases the levels of Hsp90 client proteins, including the androgen receptor (AR), bcr-abl, epidermal growth factor (EGFR), Raf-1, and FLT3 (FIG. 3). Furthermore, celastrol, gedunin, and derivatives thereof as shown in FIG. 1 inhibit downstream signaling and growth mediated by the Hsp90 client AR in a prostate cancer cell line (FIG. 4). Celastrol has also been reported to induce a heat shock response, a hallmark of Hsp90 inhibition. Hsp27 and Hsp90 expression is induced upon celastrol treatment. These data demonstrate that celastrol acts as an Hsp90 inhibitor.

Hsp90 inhibitors are useful as cancer therapies. Both existing and novel Hsp90 inhibitors are of notable interest as cancer therapies because of their ability to repress activity of multiple oncogenic pathways. Cancer cells have been shown to be more sensitive to Hsp90 inhibitors than non-malignant cells due to increased intracellular Hsp90 inhibitor levels and increased sensitivity of oncogenic mutants of key proteins. Celastrol, gedunin, and derivatives thereof as described herein are useful in the treatment of proliferative diseases such as cancers (e.g., prostate cancer, leukemia, lung cancer, etc.). Celastrol, gedunin, and derivatives thereof may be combined with other anti-cancer therapies in the treatment of cancer.

### Compounds of the Invention

Celastrol is a quinone methide triterpene found in the plant Trypterigium wilfordii and other Celastraceae family members. Celastrol derivatives include dihydrocelastrol, pristimerol, dihydrocelastrol diacetate, and celastrol methyl ester as well as other compounds described herein. Celastrol and Celastraceae extracts have a history of safe and effective use in vivo. Extracts containing celastrol have been used as a traditional Chinese therapy in humans without reports of significant limiting side effects. The major chronic toxicity in rats at 30 mg/kg extract was azoospermia and decreased testicular weight, though this may result from other extract components than celastrol. Purified celastrol showed significant bioactivity in mouse models of arthritis when administered at 1-3 mg/kg daily; similarly, it showed activity at 7 mg/kg daily in rat models for Alzheimer's disease.

In one aspect, compounds of the invention include celastrol derivatives of formula:

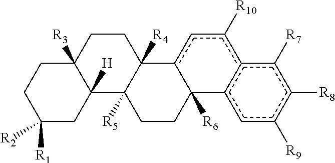

wherein

each dashed line independently represents either the presence or absence of a bond;

R₁ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{A}; —C(═O)R_{A}; —CHO; —CO₂H; —CO₂R_{A}; —CN; —SCN; —SR_{A}; —SOR_{A}; —SO₂R_{A}; —NO₂; —N₃; —NH₂; —NHR_{A}; —N(R_{A})₂; —NHC(═O)R_{A}; —NR_{A}C(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₂ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{B}; —C(═O)R_{B}; —CHO; —CO₂H; —CO₂R_{B}; —CN; —SCN; —SR_{B}; —SOR_{B}; —SO₂R_{B}; —NO₂; —N₃; —NH₂; —NHR_{B}; —N(R_{B})₂; —NHC(═O)R_{B}; —NR_{B}C(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₃ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{C}; —C(═O)R_{C}; —CHO; —CO₂H; —CO₂R_{C}; —CN; —SCN; —SR_{C}; —SOR_{C}; —SO₂R_{C}; —NO₂; —N₃; —NH₂; —NHR_{C}; —N(R_{C})₂; —NHC(═O)R_{C}; —NR_{C}C(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₄ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{D}; —C(═O)R_{D}; —CHO; —CO₂H; —CO₂R_{D}; —CN; —SCN; —SR_{D}; —SOR_{D}; —SO₂R_{D}; —NO₂; —N₃; —NH₂; —NHR_{D}; —N(R_{D})₂; —NHC(═O)R_{D}; —NR_{D}C(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₅ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{E}; —C(═O)R_{E}; —CHO; —CO₂H; —CO₂R_{E}; —CN; —SCN; —SR_{E}; —SOR_{E}; —SO₂R_{E}; —NO₂; —N₃; —NH₂; —NHR_{E}; —N(R_{E})₂; —NHC(═O)R_{E}; —NR_{E}C(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₆ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{F}; —C(═O)R_{F}; —CHO; —CO₂H; —CO₂R_{F}; —CN; —SCN; —SR_{F}; —SOR_{F}; —SO₂R_{F}; —NO₂; —N₃; —NH₂; —NHR_{F}; —N(R_{F})₂; —NHC(═O)R_{F}; —NR_{F}C(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₇ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{G}; ═O; —C(═O)R_{G}; —CHO; —CO₂H; —CO₂R_{G}; —CN; —SCN; —SR_{G}; —SOR_{G}; —SO₂R_{G}; —NO₂; —N₃; —NH₂; —NHR_{G}; —N(R_{G})₂; —NHC(═O)R_{G}; —NR_{G}C(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₈ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{H}; ═O; —C(═O)R_{H}; —CHO; —CO₂H; —CO₂R_{H}; —CN; —SCN; —SR_{H}; —SOR_{H}; —SO₂R_{H}; —NO₂; —N₃; —NH₂; —NHR_{H}; —N(R_{H})₂; —NHC(═O)R_{H}; —NR_{H}C(═O)R_{H}; —NR_{H}C(═O)N(R_{H})₂; —OC(═O)OR_{H}; —OC(═O)R_{H}; —OC(═O)N(R_{H})₂; —NR_{H}C(═O)OR_{H}; or —C(R_{H})₃; wherein each occurrence of R_{H} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₉ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{I}; ═O; —C(═O)R_{I}; —CHO; —CO₂H; —CO₂R_{I}; —CN; —SCN; —SR_{I}; —SOR_{I}; —SO₂R_{I}; —NO₂; —N₃; —NH₂; —NHR_{I}; —N(R_{I})₂; —NHC(═O)R_{I}; —NR_{I}C(═O)R_{I}; —NR_{I}C(═O)N(R_{I})₂; —OC(═O)OR_{I}; —OC(═O)R_{I}; —OC(═O)N(R)₂; —NR_{I}C(═O)OR_{I}; or —C(R_{I})₃; wherein each occurrence of R_{I} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₁₀ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{J}; ═O; —C(═O)R_{J}; —CHO; —CO₂H; —CO₂R_{J}; —CN; —SCN; —SR_{J}; —SOR_{J}; —SO₂R_{J}; —NO₂; —N₃; —NH₂; —NHR_{I}; —N(R_{J})₂; —NHC(═O)R_{J}; —NR_{J}C(═O)R_{J}; —NR_{J}C(═O)N(R_{J})₂; —OC(═O)OR_{J}; —OC(═O)R_{J}; —OC(═O)N(R_{J})₂; —NR_{I}C(═O)OR_{J}; or —C(R_{J})₃; wherein each occurrence of R_{J} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety; and pharmaceutically acceptable salts, stereoisomers, tautomers, and pro-drugs thereof.

In certain embodiments, R₁ is hydrogen. In certain embodiment, R₁ is cyclic or acyclic, substituted or unsubstituted, branched or unbranched aliphatic. In other embodiments, R₁ is acyclic, substituted or unsubstituted, branched or unbranched aliphatic. In certain embodiments, R₁ is C₁-C₆ aliphatic. In other embodiments, R₁ is C₁-C₆ alkyl. In certain embodiments, R₁ is methyl, ethyl, iso-propyl, or n-propyl. In certain specific embodiments, R₁ is methyl. In certain embodiments, R₁ is substituted methyl. In certain embodiments, R₁ is not methyl.

In certain embodiments, R₂ is substituted or unsubstituted, branched or unbranched acyl. In certain embodiments, R₂ is unsubstituted, unbranched acyl. In certain embodiments, R₂ is —CO₂H. In other embodiments, R₂ is —C(═O)OR_{B}. In certain embodiments, R₂ is —C(═O)OMe. In other embodiments, R₂ is —C(═O)NHR_{B}. In yet other embodiments, R₂ is —C(═O)N(R_{B})₂. In yet other embodiments, R₂ is —CH₂OH. In other embodiments, R₂ is —CHO. In certain embodiment, R₂ is cyclic or acyclic, substituted or unsubstituted, branched or unbranched aliphatic. In other embodiments, R₂ is acyclic, substituted or unsubstituted, branched or unbranched aliphatic. In certain embodiments, R₂ is C₁-C₆ aliphatic. In other embodiments, R₂ is C₁-C₆ alkyl. In certain embodiments, R₂ is methyl, ethyl, iso-propyl, or n-propyl. In certain specific embodiments, R₂ is methyl. In certain embodiments, R₂ is substituted methyl.

In certain embodiments, R₃ is hydrogen. In certain embodiment, R₃ is cyclic or acyclic, substituted or unsubstituted, branched or unbranched aliphatic. In other embodiments, R₃ is acyclic, substituted or unsubstituted, branched or unbranched aliphatic. In certain embodiments, R₃ is C₁-C₆ aliphatic. In other embodiments, R₃ is C₁-C₆ alkyl. In certain embodiments, R₃ is methyl, ethyl, iso-propyl, or n-propyl. In certain specific embodiments, R₃ is methyl. In certain embodiments, R₃ is substituted methyl. In certain embodiments, R₃ is not methyl.

In certain embodiments, R₄ is hydrogen. In certain embodiment, R₄ is cyclic or acyclic, substituted or unsubstituted, branched or unbranched aliphatic. In other embodiments, R₄ is acyclic, substituted or unsubstituted, branched or unbranched aliphatic. In certain embodiments, R₄ is C₁-C₆ aliphatic. In other embodiments, R₄ is C₁-C₆ alkyl. In certain embodiments, R₄ is methyl, ethyl, iso-propyl, or n-propyl. In certain specific embodiments, R₄ is methyl. In certain embodiments, R₄ is substituted methyl. In certain embodiments, R₄ is not methyl.

In certain embodiments, R₅ is hydrogen. In certain embodiment, R₅ is cyclic or acyclic, substituted or unsubstituted, branched or unbranched aliphatic. In other embodiments, R₅ is acyclic, substituted or unsubstituted, branched or unbranched aliphatic. In certain embodiments, R₅ is C₁-C₆ aliphatic. In other embodiments, R₅ is C₁-C₆ alkyl. In certain embodiments, R₅ is methyl, ethyl, iso-propyl, or n-propyl. In certain specific embodiments, R₅ is methyl. In certain embodiments, R₅ is substituted methyl. In certain embodiments, R₅ is not methyl.

In certain embodiments, R₆ is hydrogen. In certain embodiment, R₆ is cyclic or acyclic, substituted or unsubstituted, branched or unbranched aliphatic. In other embodiments, R₆ is acyclic, substituted or unsubstituted, branched or unbranched aliphatic. In certain embodiments, R₆ is C₁-C₆ aliphatic. In other embodiments, R₆ is C₁-C₆ alkyl. In certain embodiments, R₆ is methyl, ethyl, iso-propyl, or n-propyl. In certain specific embodiments, R₆ is methyl. In certain embodiments, R₆ is substituted methyl. In certain embodiments, R₆ is not methyl.

In certain embodiments, R₇ is hydrogen. In certain embodiment, R₇ is cyclic or acyclic, substituted or unsubstituted, branched or unbranched aliphatic. In other embodiments, R₇ is acyclic, substituted or unsubstituted, branched or unbranched aliphatic. In certain embodiments, R₇ is C₁-C₆ aliphatic. In other embodiments, R₇ is C₁-C₆ alkyl. In certain embodiments, R₇ is methyl, ethyl, iso-propyl, or n-propyl. In certain specific embodiments, R₇ is methyl. In certain embodiments, R₇ is substituted methyl. In certain embodiments, R₇ is not methyl.

In certain embodiments, R₈ is cyclic or acyclic, substituted or unsubstituted, branched or unbranched heteroaliphatic. In certain embodiments, R₈ is —OR_{H}. In certain embodiments, R₈ is —OH. In other embodiments, R₈ is ═O. In other embodiments, R₈ is —OC(═O)R_{H}. In other embodiments, R₈ is —OC(═O)OR_{H}. In other embodiments, R₈ is —OC(═O)NHR_{H}. In other embodiments, R₈ is —OC(═O)CH₃. In yet other embodiments, R_{H} is an oxygen protecting group. In certain embodiment, R₈ is cyclic or acyclic, substituted or unsubstituted, branched or unbranched aliphatic. In other embodiments, R₅ is acyclic, substituted or unsubstituted, branched or unbranched aliphatic. In certain embodiments, R₈ is C₁-C₆ aliphatic. In other embodiments, R₈ is C₁-C₆ alkyl. In certain embodiments, R₈ is methyl, ethyl, iso-propyl, or n-propyl. In certain specific embodiments, R₈ is methyl. In certain embodiments, R₈ is substituted methyl:

In certain embodiments, R₉ is hydrogen. In certain embodiments, R₉ is cyclic or acyclic, substituted or unsubstituted, branched or unbranched heteroaliphatic. In certain embodiments, R₉ is ═O. In certain embodiments, R₉ is —OR_{I}. In certain embodiments, R₉ is —OH. In other embodiments, R₉ is —OC(═O)R_{I}. In other embodiments, R₉ is —OC(═O)OR_{I}. In other embodiments, R₉ is —OC(═O)NHR_{I}. In other embodiments, R₉ is —OC(═O)CH₃. In yet other embodiments, R_{I} is an oxygen protecting group. In certain embodiment, R₉ is cyclic or acyclic, substituted or unsubstituted, branched or unbranched aliphatic. In other embodiments, R₉ is acyclic, substituted or unsubstituted, branched or unbranched aliphatic. In certain embodiments, R₉ is C₁-C₆ aliphatic. In other embodiments, R₉ is C₁-C₆ alkyl. In certain embodiments, R₉ is methyl, ethyl, iso-propyl, or n-propyl. In certain specific embodiments, R₉ is methyl. In certain embodiments, R₉ is substituted methyl.

In certain embodiments, R₁₀ is hydrogen. In certain embodiments, R₁₀ is cyclic or acyclic, substituted or unsubstituted, branched or unbranched heteroaliphatic. In certain embodiments, R₁₀ is —N(R_{J})₂. In certain embodiments, R₁₀ is —SR_{J}. In certain embodiments, R₁₀ is —OR_{J}. In certain embodiments, R₁₀ is —OH. In other embodiments, R₁₀ is —OC(═O)R_{J}. In other embodiments, R₁₀ is —OC(═O)OR_{J}. In other embodiments, R₁₀ is —OC(═O)NHR_{J}. In other embodiments, R₁₀ is —OC(═O)CH₃. In yet other embodiments, R_{J} is an oxygen protecting group. In certain embodiment, R₁₀ is cyclic or acyclic, substituted or unsubstituted, branched or unbranched aliphatic. In other embodiments, R₁₀ is acyclic, substituted or unsubstituted, branched or unbranched aliphatic. In certain embodiments, R₁₀ is C₁-C₆ aliphatic. In other embodiments, R₁₀ is C₁-C₆ alkyl. In certain embodiments, R₁₀ is substituted or unsubstituted aryl. In certain embodiments, R₁₀ is substituted or unsubstituted heteroaryl.

In certain embodiments, R₁, R₃, R₄, R₅, R₆, and R₇ are all methyl. In certain embodiments, at least one of R₁, R₃, R₄, R₅, R₆, and R₇ is not methyl. In certain embodiments, R₈ is —OH, —OAc, or —OR_{H}, wherein R_{H} is an oxygen protecting group. In certain embodiments, R₉ is ═O, —OH, —OAc, or —OR₁, wherein R₁ is an oxygen protecting group. In certain embodiments, R₈ is —OH, and R₉ is ═O or —OH.

In certain embodiments, the compound is of formula:

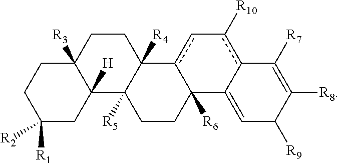

In certain embodiments, the compound is of formula:

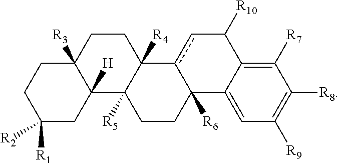

In certain embodiments, the compound is of formula:

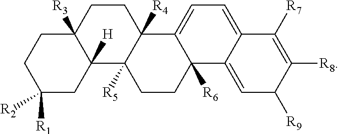

In certain embodiments, the compounds is of formula:

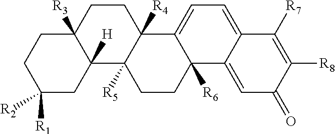

wherein R₉ is ═O.

In other embodiments, the compound is of formula:

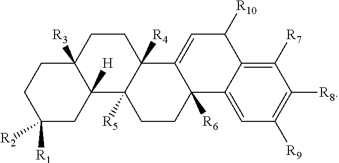

In other embodiments, the compound is of formula:

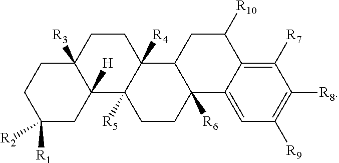

In certain embodiments, the compound is of formula:

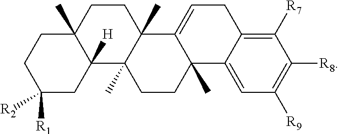

In other embodiments, the compound is of formula:

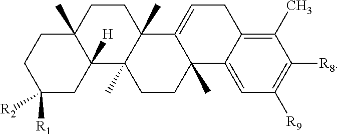

In certain embodiments, the compound is of the formula:

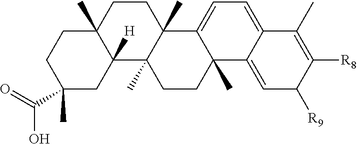

wherein

R₈ is hydroxyl (—OH) or acetyl-protected hydroxyl

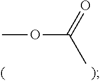

and

R₉ is oxo (═O), hydrogen (—H), or acetyl-protected hydroxyl

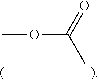

In certain embodiments, the compound is not celastrol, pristimerol, dihydrocelastrol, or dihydrocelastryl diacetate. In certain embodiments, the compound is not celastrol methyl ester.

Gedunin is a structurally similar compound isolated from plants of the Meliaceae family. Gedunin derivatives include deoxygedunin, deacetylgedunin, 7-desacetoxy-6,7-dehydrogedunin, 3-deoxo-3β-acetoxydeoxydihydrogedunin, deacetoxy-7-oxogedunin, deacetylgedunin, dihydro-7-desacetyldeoxygedunin, and 3α-hydroxydeoxodihydrogedunin as well as other compounds described herein. Celastrol, gedunin, and several of their derivatives are cell permeable and have significant activity in cell culture and in vivo.

In one aspect, compounds of the invention include gedunin derivatives of formula:

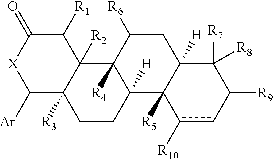

wherein

Ar is a substituted or unsubstituted aryl or heteroaryl moiety;

X is —O—, —NH—, —NR_{X}—, —CH₂—, —CHR_{X}—, or —C(R_{X})₂—, wherein R_{X} is a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; heteroaryloxy; or heteroarylthio moiety;

a dashed line represents either the presence or absence of a bond;

R₁ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{A}; —C(═O)R_{A}; —CHO; —CO₂H; —CO₂R_{A}; —CN; —SCN; —SR_{A}; —SOR_{A}; —SO₂R_{A}; —NO₂; —N₃; —NH₂; —NHR_{A}; —N(R_{A})₂; —NHC(═O)R_{A}; —NR_{A}C(═O)R_{A}; —NR_{A}C(═O)N(R_{A})₂; —OC(═O)OR_{A}; —OC(═O)R_{A}; —C(═O)N(R_{A})₂; —NR_{A}C(═O)OR_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₂ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{B}; —C(═O)R_{B}; —CHO; —CO₂H; —CO₂R_{B}; —CN; —SCN; —SR_{B}; —SOR_{B}; —SO₂R_{B}; —NO₂; —N₃; —NH₂; —NHR_{B}; N(R_{B})₂; —NHC(═O)R_{B}; —NR_{B}C(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₁ and R₂ may be taken together to form an epoxide ring, aziridine ring, cyclopropyl ring, or a bond of a carbon-carbon double bond;

R₃ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{C}; —C(═O)R_{C}; —CHO; —CO₂H; —CO₂R_{C}; —CN; —SCN; —SR_{C}; —SOR_{C}; —SO₂R_{C}; —NO₂; —N₃; —NH₂; —NHR_{C}; —N(R_{C})₂; —NHC(═O)R_{C}; —NR_{C}C(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₄ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{D}; —C(═O)R_{D}; —CHO; —CO₂H; —CO₂R_{D}; —CN; —SCN; —SR_{D}; —SOR_{D}; —SO₂R_{D}; —NO₂; —N₃; —NH₂; —NHR_{D}; —N(R_{D})₂; —NHC(═O)R_{D}; —NR_{D}C(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₅ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{E}; —C(═O)R_{E}; —CHO; —CO₂H; —CO₂R_{E}; —CN; —SCN; —SR_{E}; —SOR_{E}; —SO₂R_{E}; —NO₂; —N₃; —NH₂; —NHR_{E}; —N(R_{E})₂; —NHC(═O)R_{E}; —NR_{E}C(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₆ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{F}; —C(═O)R_{F}; —CHO; —CO₂H; —CO₂R_{F}; —CN; —SCN; —SR_{F}; —SOR_{F}; —SO₂R_{F}; —NO₂; —N₃; —NH₂; —NHR_{F}; —N(R_{F})₂; —NHC(═O)R_{F}; —NR_{F}C(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₇ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{G}; —C(═O)R_{G}; —CHO; —CO₂H; —CO₂R_{G}; —CN; —SCN; —SR_{G}; —SOR_{G}; —SO₂R_{G}; —NO₂; —N₃; —NH₂; —NHR_{G}; —N(R_{G})₂; —NHC(═O)R_{G}; —NR_{G}C(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₈ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{H}; —C(═O)R_{H}; —CHO; —CO₂H; —CO₂R_{H}; —CN; —SCN; —SR_{H}; —SOR_{H}; —SO₂R_{H}; —NO₂; —N₃; —NH₂; —NHR_{H}; —N(R_{H})₂; —NHC(═O)R_{H}; —NR_{H}C(═O)R_{H}; —NR_{H}C(═O)N(R_{H})₂; —OC(═O)OR_{H}; —OC(═O)R_{H}; —OC(═O)N(R_{H})₂; —NR_{H}C(═O)OR_{H}; or —C(R_{H})₃; wherein each occurrence of R_{H} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₉ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{I}; ═O; —C(═O)R_{I}; —CHO; —CO₂H; —CO₂R_{I}; —CN; —SCN; —SR_{I}; —SOR_{I}; —SO₂R_{I}; —NO₂; —N₃; —NH₂; —NHR_{I}; —N(R_{I})₂; —NHC(═O)R_{I}; —NR_{I}C(═O)R_{I}; —NR_{I}C(═O)N(R_{I})₂; —OC(═O)OR_{I}; —OC(═O)R_{I}; —OC(═O)N(R_{I})₂; —NR_{I}C(═O)OR_{I}; or —C(R_{I})₃; wherein each occurrence of R_{I} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;

R₁₀ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{J}; ═O; —C(═O)R_{J}; —CHO; —CO₂H; —CO₂R_{J}; —CN; —SCN; —SR_{J}; —SOR_{J}; —SO₂R_{J}; —NO₂; —N₃; —NH₂; —NHR_{I}; —N(R_{J})₂; —NHC(═O)R_{J}; —NR_{J}C(═O)R_{J}; —NR_{J}C(═O)N(R_{J})₂; —OC(═O)OR_{J}; —OC(═O)R_{J}; —OC(═O)N(R_{R})₂; —NR_{I}C(═O)OR_{J}; or —C(R_{J})₃; wherein each occurrence of R_{J} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety; and pharmaceutically acceptable salts, stereoisomers, tautomers, and pro-drugs thereof.

In certain embodiments, X is —O—. In certain other embodiments, X is —NH—.

In certain embodiments, Ar is a substituted or unsubstituted aryl moiety. In other embodiments, Ar is an unsubstituted aryl moiety. In yet other embodiments, Ar is an unsubstituted phenyl ring. In certain embodiments, Ar is a substituted or unsubstituted heteroaryl moiety. In certain embodiments, Ar is an unsubstituted aryl moiety. In certain embodiments, Ar is a five-membered heteroaryl moiety. In other embodiments, Ar is a six-membered heteroaryl moiety. In certain embodiments, Ar is a furanyl moiety.

In certain embodiments, R₁ is hydrogen. In certain embodiments, R₁ is —OH. In other embodiments, R₁ is —OR_{A}. In other embodiments, R₁ is —OC(═O)R_{A}. In other embodiments, R₁ is —OC(═O)OR_{A}. In other embodiments, R₁ is —OC(═O)NHR_{A}. In other embodiments, R₁ is —OC(═O)CH₃.

In certain embodiments, R₂ is hydrogen. In certain embodiments, R₂ is —OH. In other embodiments, R₂ is —OR_{B}. In other embodiments, R₂ is —OC(═O)R_{B}. In other embodiments, R₂ is —OC(═O)OR_{B}. In other embodiments, R₂ is —OC(═O)NHR_{B}. In other embodiments, R₂ is —OC(═O)CH₃.

In certain embodiments, R₁ and R₂ together form an epoxide ring. In other embodiments, R₁ and R₂ together form a cyclopropyl ring. In yet other embodiments, R₁ and R₂ together form an aziridine ring. In yet other embodiments, R₁ and R₂ together form a bond of a carbon-carbon double bond.

In certain embodiments, R₃ is hydrogen. In certain embodiment, R₃ is cyclic or acyclic, substituted or unsubstituted, branched or unbranched aliphatic. In other embodiments, R₃ is acyclic, substituted or unsubstituted, branched or unbranched aliphatic. In certain embodiments, R₃ is C₁-C₆ aliphatic. In other embodiments, R₃ is C₁-C₆ alkyl. In certain embodiments, R₃ is methyl, ethyl, iso-propyl, or n-propyl. In certain specific embodiments, R₃ is methyl. In certain embodiments, R₃ is substituted methyl. In certain embodiments, R₃ is not methyl.

In certain embodiments, R₄ is hydrogen. In certain embodiment, R₄ is cyclic or acyclic, substituted or unsubstituted, branched or unbranched aliphatic. In other embodiments, R₄ is acyclic, substituted or unsubstituted, branched or unbranched aliphatic. In certain embodiments, R₄ is C₁-C₆ aliphatic. In other embodiments, R₄ is C₁-C₆ alkyl. In certain embodiments, R₄ is methyl, ethyl, iso-propyl, or n-propyl. In certain specific embodiments, R₄ is methyl. In certain embodiments, R₄ is substituted methyl. In certain embodiments, R4₃ is not methyl.

In certain embodiments, R₅ is hydrogen. In certain embodiment, R₅ is cyclic or acyclic, substituted or unsubstituted, branched or unbranched aliphatic. In other embodiments, R₅ is acyclic, substituted or unsubstituted, branched or unbranched aliphatic. In certain embodiments, R₅ is C₁-C₆ aliphatic. In other embodiments, R₅ is C₁-C₆ alkyl. In certain embodiments, R₅ is methyl, ethyl, iso-propyl, or n-propyl. In certain specific embodiments, R₅ is methyl. In certain embodiments, R₅ is substituted methyl. In certain embodiments, R₅ is not methyl.

In certain embodiments, R₆ is hydrogen. In certain embodiments, R₆ is cyclic or acyclic, substituted or unsubstituted, branched or unbranched heteroaliphatic. In certain embodiments, R₆ is ═O. In certain embodiments, R₆ is —OR_{F}. In certain embodiments, R₆ is —OH. In other embodiments, R₆ is —OC(═O)R_{F}. In other embodiments, R₆ is —OC(═O)OR_{F}. In other embodiments, R₆ is —OC(═O)NHR_{F}. In other embodiments, R₆ is —OC(═O)CH₃. In yet other embodiments, R₆ is —OR_{F}, wherein R_{F} is an oxygen protecting group. In certain embodiment, R₆ is cyclic or acyclic, substituted or unsubstituted, branched or unbranched aliphatic. In other embodiments, R₆ is acyclic, substituted or unsubstituted, branched or unbranched aliphatic. In certain embodiments, R₆ is C₁-C₆ aliphatic. In other embodiments, R₆ is C₁-C₆ alkyl. In certain embodiments, R₆ is methyl, ethyl, iso-propyl, or n-propyl. In certain specific embodiments, R₆ is methyl. In certain embodiments, R₆ is substituted methyl.

In certain embodiments, R₇ is hydrogen. In certain embodiment, R₇ is cyclic or acyclic, substituted or unsubstituted, branched or unbranched aliphatic. In other embodiments, R₇ is acyclic, substituted or unsubstituted, branched or unbranched aliphatic. In certain embodiments, R₇ is C₁-C₆ aliphatic. In other embodiments, R₇ is C₁-C₆ alkyl. In certain embodiments, R₇ is methyl, ethyl, iso-propyl, or n-propyl. In certain specific embodiments, R₇ is methyl. In certain embodiments, R₇ is substituted methyl. In certain embodiments, R₇ is not methyl.

In certain embodiments, R₈ is hydrogen. In certain embodiment, R₈ is cyclic or acyclic, substituted or unsubstituted, branched or unbranched aliphatic. In other embodiments, R₈ is acyclic, substituted or unsubstituted, branched or unbranched aliphatic. In certain embodiments, R₈ is C₁-C₆ aliphatic. In other embodiments, R₈ is C₁-C₆ alkyl. In certain embodiments, R₈ is methyl, ethyl, iso-propyl, or n-propyl. In certain specific embodiments, R₈ is methyl. In certain embodiments, R₈ is substituted methyl. In certain embodiments, R₈ is not methyl.

In certain embodiments, R₉ is hydrogen. In certain embodiments, R₉ is cyclic or acyclic, substituted or unsubstituted, branched or unbranched heteroaliphatic. In certain embodiments, R₉ is ═O. In certain embodiments, R₉ is —OR. In certain embodiments, R₉ is —OH. In other embodiments, R₉ is —OC(═O)R_{I}. In other embodiments, R₉ is —OC(═O)OR_{I}. In other embodiments, R₉ is —OC(═O)NHR_{I}. In other embodiments, R₉ is —OC(═O)CH₃. In yet other embodiments, R₉ is —OR_{I}, wherein R_{I} is an oxygen protecting group. In certain embodiment, R₉ is cyclic or acyclic, substituted or unsubstituted, branched or unbranched aliphatic. In other embodiments, R₉ is acyclic, substituted or unsubstituted, branched or unbranched aliphatic. In certain embodiments, R₉ is C₁-C₆ aliphatic. In other embodiments, R₉ is C₁-C₆ alkyl. In certain embodiments, R₉ is methyl, ethyl, iso-propyl, or n-propyl. In certain specific embodiments, R₉ is methyl. In certain embodiments, R₉ is substituted methyl.

In certain embodiments, R₁₀ is hydrogen. In certain embodiments, R₁₀ is cyclic or acyclic, substituted or unsubstituted, branched or unbranched heteroaliphatic. In certain embodiments, R₁₀ is —N(R_{J})₂. In certain embodiments, R₁₀ is —SR_{J}. In certain embodiments, R₁₀ is —OR_{J}. In certain embodiments, R₁₀ is —OH. In other embodiments, R₁₀ is —OC(═O)R_{J}. In other embodiments, R₁₀ is —OC(═O)OR_{J}. In other embodiments, R₁₀ is —OC(═O)NHR_{J}. In other embodiments, R₁₀ is —OC(═O)CH₃. In yet other embodiments, R_{J} is an oxygen protecting group. In certain embodiment, R₁₀ is cyclic or acyclic, substituted or unsubstituted, branched or unbranched aliphatic. In other embodiments, R₁₀ is acyclic, substituted or unsubstituted, branched or unbranched aliphatic. In certain embodiments, R₁₀ is C₁-C₆ aliphatic. In other embodiments, R₁₀ is C₁-C₆ alkyl. In certain embodiments, R₁₀ is substituted or unsubstituted aryl. In certain embodiments, R₁₀ is substituted or unsubstituted heteroaryl.

In certain embodiments, the dashed line represents the absence of a bond. In other embodiments, the dashed line represents a bond of a carbon-carbon double bond.

In certain embodiments, R₃, R₄, R₅, R₇, and R₈ are all methyl. In certain embodiments, at least one of R₃, R₄, R₅, R₇, and R₈ is not methyl.

In certain embodiments, the compound is of formula:

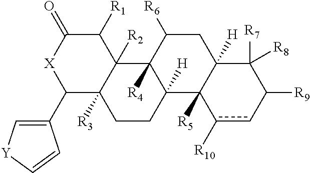

wherein

Y is —O—, —S—, —NH—, or —NR_{Y}—, wherein R_{Y} is a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; heteroaryloxy; or heteroarylthio moiety.

In certain embodiment, the compounds is of formula:

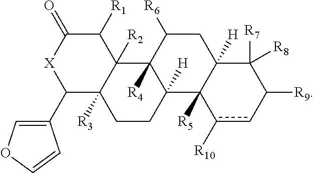

In certain embodiments, the compound is of formula:

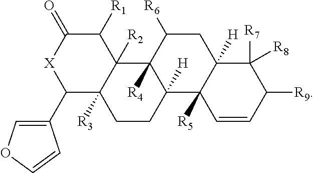

In other embodiments, the compound is of formula:

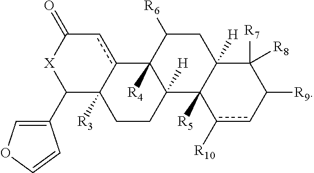

In yet other embodiments, the compound is of formula:

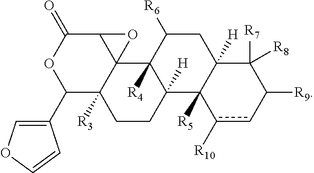

In certain embodiments, the compound is of the formula:

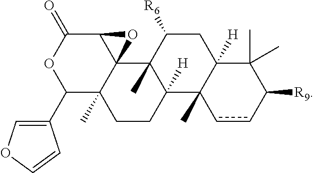

In certain embodiments, the compound is of the formula:

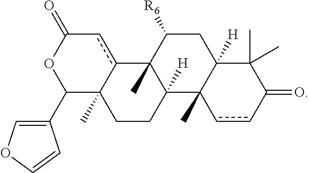

In certain other embodiments, the compound is of the formula:

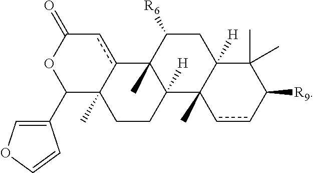

In certain embodiments, the compound is of the formula:

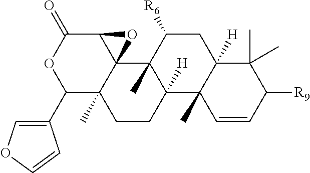

wherein

R₆ is hydrogen (—H); oxo (═O), hydroxyl (—OH), or acetyl-protected hydroxyl

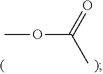

and

R₉ is oxo (═O), or acetyl-protected hydroxyl

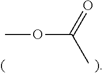

In certain embodiments, the compound is not gedunin, deoxygedunin, deacetylgedunin, 3α-hydroxydeoxodihydrogedunin, deacetoxy-7-oxogedunin, 3-deoxo-3β-acetoxydeoxydehydrogedunin, 7-desacetoxy-6,7-dehydrogedunin, dihydro-7-desacetyldeoxygedunin, or deacetylgedunin.

Certain of compounds described above are natural products, e.g., celastrol and gedunin. These compounds therefore may be purified from their natural state. Preferably, the natural product is isolated from at least one component of its natural state. In certain embodiments, the compound is at least 75%, 80%, 90%, 95%, 98%, or 99% pure. For compounds prepared synthetically or semi-synthetically, the compounds is typically purified from intermediates, side products, starting materials, catalysts, ligands, etc. found in a reaction mixture. In certain embodiments, the compound is at least 75%, 80%, 90%, 95%, 98%, or 99% pure.

Some of the foregoing compounds comprise one or more asymmetric centers, and thus can exist in various isomeric forms, e.g., stereoisomers and/or diastereomers. Thus, inventive compounds and pharmaceutical compositions thereof may be in the form of an individual enantiomer, diastereomer or geometric isomer, or may be in the form of a mixture of stereoisomers. In certain embodiments, the compounds of the invention are enantiopure compounds. In certain other embodiments, mixtures of stereoisomers or diastereomers are provided.

Furthermore, certain compounds, as described herein may have one or more double bonds that can exist as either the Z or E isomer, unless otherwise indicated. The invention additionally encompasses the compounds as individual isomers substantially free of other isomers and alternatively, as mixtures of various isomers, e.g., racemic mixtures of stereoisomers. In addition to the above-mentioned compounds per se, this invention also encompasses pharmaceutically acceptable derivatives of these compounds and compositions comprising one or more compounds of the invention and one or more pharmaceutically acceptable excipients or additives.

Compounds of the invention may be prepared by crystallization of compound of any of the formula above under different conditions and may exist as one or a combination of polymorphs of compound of any general formula above forming part of this invention. For example, different polymorphs may be identified and/or prepared using different solvents, or different mixtures of solvents for recrystallization; by performing crystallizations at different temperatures; or by using various modes of cooling, ranging from very fast to very slow cooling during crystallizations. Polymorphs may also be obtained by heating or melting the compound followed by gradual or fast cooling. The presence of polymorphs may be determined by solid probe NMR spectroscopy, IR spectroscopy, differential scanning calorimetry, powder X-ray diffractogram and/or other techniques. Thus, the present invention encompasses inventive compounds, their derivatives, their tautomeric forms, their stereoisomers, their polymorphs, their pharmaceutically acceptable salts their pharmaceutically acceptable solvates and pharmaceutically acceptable compositions containing them.

### Preparation of the Compounds

Some of the compounds described herein are natural products. For example, celastrol and gedunin are both natural products which can be isolated from the plants that produce them. Other derivatives of celastrol and gedunin are also available by natural products isolation. Using techniques known in the art of natural products isolation including solvent extraction, column chromatography, HPLC, crystallization, etc., these natural products may be purified to the desired state of purity needed for desired use of the compounds. These natural products may also be obtained by total chemical synthesis.

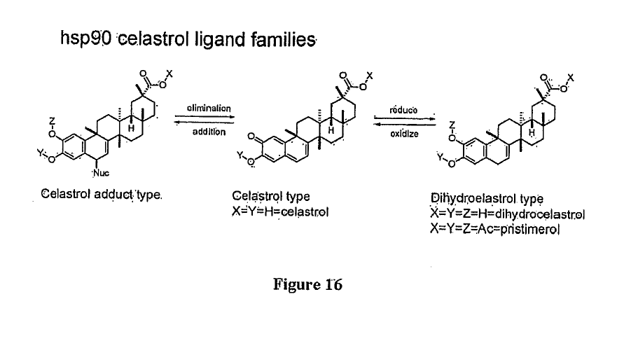

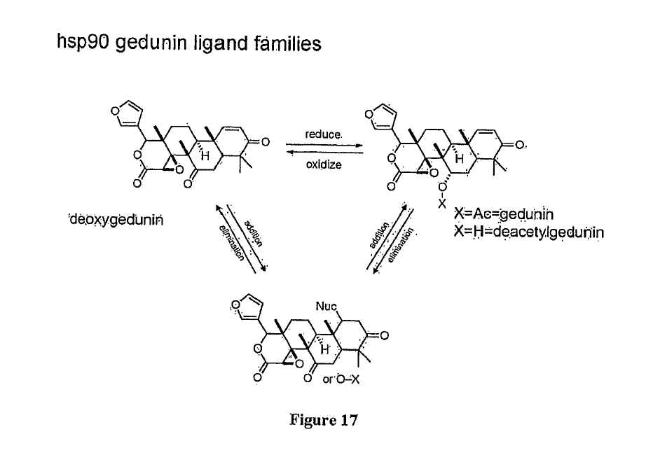

Certain compounds of the invention are derivatives of the natural products celastrol and gedunin. These compounds may be prepared by total synthesis or by semi-synthesis. See, e.g., FIGS. 16 and 17. As would be appreciated by one of skill in this art, the compounds may be prepared by modifying functional groups of the natural product. For example, hydroxyl groups of the natural product may be alkylated, acylated, reduced, or oxidized using synthetic techniques known in the art. Carbonyl groups may be reduced or oxidized. Acyl groups may be removed, reduced, hydrolyzed, trans-esterified, trans-amidated, oxidized, etc. The unsaturated functional groups of the natural product such as carbon-carbon double bonds may be reduced, expoxidized, hydroxylated, oxidized, cyclopronated, alkylated, etc. Various functional group transformations useful in the preparation of the compounds of the invention are described in Smith and March, March's Advanced Organic Chemistry (5^{th} Ed.), New York: John Wiley & Sons, Inc., 2001; and Larock, Comprehensive Organic Transformations, New York: VCH Publishers, Inc., 1989; Carruthers, Some Modern Methods of Organic Synthesis 3^{rd} Ed., Cambridge University Press, 1992; each of which is incorporated herein by reference. Derivatives of celastrol and gedunin may also be prepared by the addition of nucleophiles at electrophilic positions of the molecule. For example, the 1,2-addition, 1,4-addition, or 1,6-addition of a nucleophile to a carbonyl or an unsaturated carbonyl system.

### Pharmaceutical Compositions

As discussed above, the present invention provides novel compounds having antitumor, antibiotic, and/or antiproliferative activity, and thus the inventive compounds are useful for the treatment of cancer, benign tumors, inflammatory diseases (e.g., autoimmune diseases), and infectious diseases.

Accordingly, in another aspect of the present invention, pharmaceutical compositions are provided, which comprise any one of the compounds described herein (or a prodrug, pharmaceutically acceptable salt or other pharmaceutically acceptable derivative thereof), and optionally comprise a pharmaceutically acceptable excipient. In certain embodiments, these compositions optionally further comprise one or more additional therapeutic agents. Alternatively, a compound of this invention may be administered to a patient in need thereof in combination with the administration of one or more other therapeutic agents. For example, additional therapeutic agents for conjoint administration or inclusion in a pharmaceutical composition with a compound of this invention may be an approved chemotherapeutic agent, or it may be any one of a number of agents undergoing approval in the Food and Drug Administration that ultimately obtain approval for the treatment of fungal infections and/or any disorder associated with cellular hyperproliferation. In certain other embodiments, the additional therapeutic agent is an anticancer agent, as discussed in more detail herein. In certain embodiments, the additional therapeutic agent is an Hsp90 inhibitor (e.g., geldanamycin, 17-AAG, monorden (a.k.a., radicicol), IPI-504, DMAG, and novobiocin). In certain other embodiments, the compositions of the invention are useful for the treatment of fungal infections.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a pro-drug or other adduct or derivative of a compound of this invention which upon administration to a patient in need is capable of providing, directly or indirectly, a compound as otherwise described herein, or a metabolite or residue thereof.

As used herein, the term “pharmaceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically acceptable salts of amines, carboxylic acids, and other types of compounds, are well known in the art. For example, Berge et al. describe pharmaceutically acceptable salts in detail in J. Pharmaceutical Sciences, 66:1-19 (1977), incorporated herein by reference. The salts can be prepared in situ during the final isolation and purification of the compounds of the invention, or separately by reacting a free base or free acid function with a suitable reagent, as described generally below. For example, a free base function can be reacted with a suitable acid. Furthermore, where the compounds of the invention carry an acidic moiety, suitable pharmaceutically acceptable salts thereof may, include metal salts such as alkali metal salts, e.g. sodium or potassium salts; and alkaline earth metal salts, e.g. calcium or magnesium salts. Examples of pharmaceutically acceptable, non-toxic acid addition salts are salts of an amino group formed with inorganic acids such as hydrochloric acid, hydrobromic acid, phosphoric acid, sulfuric acid, and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, loweralkyl sulfonate and aryl sulfonate.

Additionally, as used herein, the term “pharmaceutically acceptable ester” refers to esters that hydrolyze in vivo and include those that break down readily in the human body to leave the parent compound or a salt thereof. Suitable ester groups include, for example, those derived from pharmaceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates, and ethylsuccinates.

Furthermore, the term “pharmaceutically acceptable prodrugs” as used herein refers to those prodrugs of the compounds of the present invention which are, within the scope of sound medical judgment, suitable for use in contact with the issues of humans and lower animals with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood. A thorough discussion is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

As described above, the pharmaceutical compositions of the present invention additionally comprise a pharmaceutically acceptable carrier, which, as used herein, includes any and all solvents, diluents, or other liquid vehicle, dispersion or suspension aids, surface active agents, isotonic agents, thickening or emulsifying agents, preservatives, solid binders, lubricants and the like, as suited to the particular dosage form desired. Remington's Pharmaceutical Sciences, Sixteenth Edition, E. W. Martin (Mack Publishing Co., Easton, Pa., 1980) discloses various carriers used in formulating pharmaceutical compositions and known techniques for the preparation thereof. Except insofar as any conventional carrier medium is incompatible with the compounds of the invention, such as by producing any undesirable biological effect or otherwise interacting in a deleterious manner with any other component(s) of the pharmaceutical composition, its use is contemplated to be within the scope of this invention. Some examples of materials which can serve as pharmaceutically acceptable carriers include, but are not limited to, sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatine; talc; excipients such as cocoa butter and suppository waxes; oils such as peanut oil, cottonseed oil; safflower oil, sesame oil; olive oil; corn oil and soybean oil; glycols; such as propylene glycol; esters such as ethyl oleate and ethyl laurate; agar; buffering agents such as magnesium hydroxide and aluminum hydroxide; alginic acid; pyrogenfree water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of the formulator.

Liquid dosage forms for oral administration include, but are not limited to, pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof. Besides inert diluents, the oral compositions can also include adjuvants such as wetting agents, emulsifying and suspending agents, sweetening, flavoring, and perfuming agents.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic parenterally acceptable diluent or solvent, for example, as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

The injectable formulations can be sterilized, for example, by filtration through a bacterial-retaining filter, or by incorporating sterilizing agents in the form of sterile solid compositions which can be dissolved or dispersed in sterile water or other sterile injectable medium prior to use.

In order to prolong the effect of a drug, it is often desirable to slow the absorption of the drug from subcutaneous or intramuscular injection. This may be accomplished by the use of a liquid suspension or crystalline or amorphous material with poor water solubility. The rate of absorption of the drug then depends upon its rate of dissolution that, in turn, may depend upon crystal size and crystalline form. Alternatively, delayed absorption of a parenterally administered drug form is accomplished by dissolving or suspending the drug in an oil vehicle. Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include (poly(orthoesters) and poly(anhydrides). Depot injectable formulations are also prepared by entrapping the drug in liposomes or microemulsions which are compatible with body tissues.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar—agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like. The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions that can be used include polymeric substances and waxes. Solid compositions of a similar type may also be employed as fillers in soft and hard-filled gelatin capsules using such excipients as lactose or milk sugar as well as high molecular weight polethylene glycols and the like.

The active compounds can also be in micro-encapsulated form with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound may be admixed with at least one inert diluent such as sucrose, lactose and starch. Such dosage forms may also comprise, as in normal practice, additional substances other than inert diluents, e.g., tableting lubricants and other tableting aids such as magnesium stearate and microcrystalline cellulose. In the case of capsules, tablets and pills, the dosage forms may also comprise buffering agents. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The present invention encompasses pharmaceutically acceptable topical formulations of inventive compounds. The term “pharmaceutically acceptable topical formulation,” as used herein, means any formulation which is pharmaceutically acceptable for intradermal administration of a compound of the invention by application of the formulation to the epidermis. In certain embodiments of the invention, the topical formulation comprises a carrier system. Pharmaceutically effective carriers include, but are not limited to, solvents (e.g., alcohols, poly alcohols, water), creams, lotions, ointments, oils, plasters, liposomes, powders, emulsions, microemulsions, and buffered solutions (e.g., hypotonic or buffered saline) or any other carrier known in the art for topically administering pharmaceuticals. A more complete listing of art-known carriers is provided by reference texts that are standard in the art, for example, Remington's Pharmaceutical Sciences, 16th Edition, 1980 and 17th Edition, 1985, both published by Mack Publishing Company, Easton, Pa., the disclosures of which are incorporated herein by reference in their entireties. In certain other embodiments, the topical formulations of the invention may comprise excipients. Any pharmaceutically acceptable excipient known in the art may be used to prepare the inventive pharmaceutically acceptable topical formulations. Examples of excipients that can be included in the topical formulations of the invention include, but are not limited to, preservatives, antioxidants, moisturizers, emollients, buffering agents, solubilizing agents, other penetration agents, skin protectants, surfactants, and propellants, and/or additional therapeutic agents used in combination to the inventive compound. Suitable preservatives include, but are not limited to, alcohols, quaternary amines, organic acids, parabens, and phenols. Suitable antioxidants include, but are not limited to, ascorbic acid and its esters, sodium bisulfite, butylated hydroxytoluene, butylated hydroxyanisole, tocopherols, and chelating agents like EDTA and citric acid. Suitable moisturizers include, but are not limited to, glycerine, sorbitol, polyethylene glycols, urea, and propylene glycol. Suitable buffering agents for use with the invention include, but are not limited to, citric, hydrochloric, and lactic acid buffers. Suitable solubilizing agents include, but are not limited to, quaternary ammonium chlorides, cyclodextrins, benzyl benzoate, lecithin, and polysorbates. Suitable skin protectants that can be used in the topical formulations of the invention include, but are not limited to, vitamin E oil, allatoin, dimethicone, glycerin, petrolatum, and zinc oxide.

In certain embodiments, the pharmaceutically acceptable topical formulations of the invention comprise at least a compound of the invention and a penetration enhancing agent. The choice of topical formulation will depend or several factors, including the condition to be treated, the physicochemical characteristics of the inventive compound and other excipients present, their stability in the formulation, available manufacturing equipment, and costs constraints. As used herein the term “penetration enhancing agent” means an agent capable of transporting a pharmacologically active compound through the stratum corneum and into the epidermis or dermis, preferably, with little or no systemic absorption. A wide variety of compounds have been evaluated as to their effectiveness in enhancing the rate of penetration of drugs through the skin. See, for example, Percutaneous Penetration Enhancers, Maibach H. I. and Smith H. E. (eds.), CRC Press, Inc., Boca Raton, Fla. (1995), which surveys the use and testing of various skin penetration enhancers, and Buyuktimkin et al., Chemical Means of Transdermal Drug Permeation Enhancement in Transdermal and Topical Drug Delivery Systems, Gosh T. K., Pfister W. R., Yum S. I. (Eds.), Interpharm Press Inc., Buffalo Grove, Ill. (1997). In certain exemplary embodiments, penetration agents for use with the invention include, but are not limited to, triglycerides (e.g., soybean oil), aloe compositions (e.g., aloe-vera gel), ethyl alcohol, isopropyl alcohol, octolyphenylpolyethylene glycol, oleic acid, polyethylene glycol 400, propylene glycol, N-decylmethylsulfoxide, fatty acid esters (e.g., isopropyl myristate, methyl laurate, glycerol monooleate, and propylene glycol monooleate) and N-methylpyrrolidone.

In certain embodiments, the compositions may be in the form of ointments, pastes, creams, lotions, gels, powders, solutions, sprays, inhalants or patches. In certain exemplary embodiments, formulations of the compositions according to the invention are creams, which may further contain saturated or unsaturated fatty acids such as stearic acid, palmitic acid, oleic acid, palmito-oleic acid, cetyl or oleyl alcohols, stearic acid being particularly preferred. Creams of the invention may also contain a non-ionic surfactant, for example, polyoxy-40-stearate. In certain embodiments, the active component is admixed under sterile conditions with a pharmaceutically acceptable carrier and any needed preservatives or buffers as may be required. Ophthalmic formulation, eardrops, and eye drops are also contemplated as being within the scope of this invention. Additionally, the present invention contemplates the use of transdermal patches, which have the added advantage of providing controlled delivery of a compound to the body. Such dosage forms are made by dissolving or dispensing the compound in the proper medium. As discussed above, penetration enhancing agents can also be used to increase the flux of the compound across the skin. The rate can be controlled by either providing a rate controlling membrane or by dispersing the compound in a polymer matrix or gel.

It will also be appreciated that the compounds and pharmaceutical compositions of the present invention can be formulated and employed in combination therapies, that is, the compounds and pharmaceutical compositions can be formulated with or administered concurrently with, prior to, or subsequent to, one or more other desired therapeutics or medical procedures. The particular combination of therapies (therapeutics or procedures) to employ in a combination regimen will take into account compatibility of the desired therapeutics and/or procedures and the desired therapeutic effect to be achieved. It will also be appreciated that the therapies employed may achieve a desired effect for the same disorder (for example, an inventive compound may be administered concurrently with another immunomodulatory agent, anticancer agent or agent useful for the treatment of psoriasis), or they may achieve different effects (e.g., control of any adverse effects).

For example, other therapies or anticancer agents that may be used in combination with the inventive compounds of the present invention include surgery, radiotherapy (in but a few examples, γ-radiation, neutron beam radiotherapy, electron beam radiotherapy, proton therapy, brachytherapy, and systemic radioactive isotopes, to name a few), endocrine therapy, biologic response modifiers (interferons, interleukins, and tumor necrosis factor (TNF) to name a few), hyperthermia and cryotherapy, agents to attenuate any adverse effects (e.g., antiemetics), and other approved chemotherapeutic drugs, including, but not limited to, alkylating drugs (mechlorethamine, chlorambucil, Cyclophosphamide, Melphalan, Ifosfamide), antimetabolites (Methotrexate), purine antagonists and pyrimidine antagonists (6-Mercaptopurine, 5-Fluorouracil, Cytarabile, Gemcitabine), spindle poisons (Vinblastine, Vincristine, Vinorelbine, Paclitaxel), podophyllotoxins (Etoposide, Irinotecan, Topotecan), antibiotics (Doxorubicin, Bleomycin, Mitomycin), nitrosoureas (Carmustine, Lomustine), inorganic ions (Cisplatin, Carboplatin), enzymes (Asparaginase), and hormones (Tamoxifen, Leuprolide, Flutamide, and Megestrol), to name a few. For a more comprehensive discussion of updated cancer therapies see, The Merck Manual, Seventeenth Ed. 1999, the entire contents of which are hereby incorporated by reference. See also the National Cancer Institute (CNI) website (www.nci.nih.gov) and the Food and Drug Administration (FDA) website for a list of the FDA approved oncology drugs (www.fda.gov/cder/cancer/druglistframe).

In certain embodiments, the pharmaceutical compositions of the present invention further comprise one or more additional therapeutically active ingredients (e.g., chemotherapeutic and/or palliative). For purposes of the invention, the term “Palliative” refers to treatment that is focused on the relief of symptoms of a disease and/or side effects of a therapeutic regimen, but is not curative. For example, palliative treatment encompasses painkillers, antinausea medications and anti-sickness drugs. In addition, chemotherapy, radiotherapy, and surgery can all be used palliatively (that is, to reduce symptoms without going for cure; e.g., for shrinking tumors and reducing pressure, bleeding, pain and other symptoms of cancer).

Additionally, the present invention provides pharmaceutically acceptable derivatives of the inventive compounds, and methods of treating a subject using these compounds, pharmaceutical compositions thereof, or either of these in combination with one or more additional therapeutic agents.

It will also be appreciated that certain of the compounds of present invention can exist in free form for treatment, or where appropriate, as a pharmaceutically acceptable derivative thereof. According to the present invention, a pharmaceutically acceptable derivative includes, but is not limited to, pharmaceutically acceptable salts, esters, salts of such esters, or a prodrug or other adduct or derivative of a compound of this invention which upon administration to a patient in need is capable of providing, directly or indirectly, a compound as otherwise described herein, or a metabolite or residue thereof.

### Research Uses, Pharmaceutical Uses, and Methods of Treatment

According to the present invention, the inventive compounds may be assayed in any of the available assays known in the art for identifying compounds having Hsp90 inhibitor activity, inhibition of protein folding, destabilization of proteins, cytotoxicity, anti-oncogenic activity, antibiotic activity, antifungal activity, and/or antiproliferative activity. For example, the assay may be cellular or non-cellular, in vivo or in vitro, high- or low-throughput format, etc.

Thus, in one aspect, compounds of this invention which are of particular interest include those which:
inhibit Hsp90 activity;
inhibit protein folding;
destabilize proteins (e.g., oncogenic proteins (e.g., BCR/ABL), receptors (e.g., androgen receptor, estrogen receptor, progesterone receptor, EGFR), protein kinases (e.g., FLT3, AKT);
destabilize receptors (e.g., androgen receptors, epidermal growth factor receptor, glucocorticoid receptor, estrogen receptor, progesterone receptor);
inhibit androgen receptor signaling in prostate cancer cells;
inhibit estrogen receptor signaling in breast cancer cells;
inhibit progesterone receptor signaling in breast cancer cells;
destabilize oncogenic proteins;
destabilize kinases;
exhibit a gene signature similar to Hsp90 inhibitors;
cause the mislocalization of proteins in the cell;
exhibit cytotoxicity;
exhibit cytotoxicity towards glucocorticoid receptor (e.g., androgen receptor) expressing cells;
inhibit the induction of the gene signature indicative of glucocorticoid stimulation (e.g., androgen, estrogen);
exhibit cytotoxic or growth inhibitory effect on cancer cell lines maintained in vitro or in animal studies using a scientifically acceptable cancer cell xenograft model; and/or
exhibit a therapeutic profile (e.g., optimum safety and curative effect) that is superior to existing chemotherapeutic agents.

As detailed in the exemplification herein, in assays to determine the ability of compounds to inhibit cancer cell growth certain inventive compounds may exhibit IC₅₀ values ≦100 μM. In certain other embodiments, inventive compounds exhibit IC₅₀ values ≦50M. In certain other embodiments, inventive compounds exhibit IC₅₀ values ≦40 μM. In certain other embodiments, inventive compounds exhibit IC₅₀ values ≦30 μM. In certain other embodiments, inventive compounds exhibit IC₅₀ values ≦20 μM. In certain other embodiments, inventive compounds exhibit IC₅₀ values ≦10 μM. In certain other embodiments, inventive compounds exhibit IC₅₀ values ≦7.5 μM. In certain embodiments, inventive compounds exhibit IC₅₀ values ≦5 μM. In certain other embodiments, inventive compounds exhibit IC₅₀ values ≦2.5 μM. In certain embodiments, inventive compounds exhibit IC₅₀ values ≦1 μM. In certain embodiments, inventive compounds exhibit IC₅₀ values ≦0.75 μM. In certain embodiments, inventive compounds exhibit IC₅₀ values ≦0.5 μM. In certain embodiments, inventive compounds exhibit IC₅₀ values ≦0.25 μM. In certain embodiments, inventive compounds exhibit IC₅₀ values ≦0.1 μM. In certain other embodiments, inventive compounds exhibit IC₅₀ values ≦75 nM. In certain other embodiments, inventive compounds exhibit IC₅₀ values ≦50 nM. In certain other embodiments, inventive compounds exhibit IC₅₀ values ≦25 nM. In certain other embodiments, inventive compounds exhibit IC₅₀ values ≦10 nM. In other embodiments, exemplary compounds exhibited IC₅₀ values ≦7.5 nM. In other embodiments, exemplary compounds exhibited IC₅₀ values ≦5 nM.

### Pharmaceutical Uses and Methods of Treatment

In general, methods of using the compounds of the present invention comprise administering to a subject in need thereof a therapeutically effective amount of a compound of the present invention. As discussed above, the compounds of the invention are inhibitors of Hsp90. Therefore, the compounds are particularly useful in treating cancer dependent upon Hsp90 for survival. Compounds of the invention may be useful in the treatment of cancers such as breast cancer, prostate cancer, ovarian cancer, lung cancer, leukemia, etc. In certain embodiments, the cancer being treated is BCR/ABL chronic myeloid leukemia, a FLT3 mutant leukemia, an EGFR mutant lung cancer, or an AKT mutant cancer. The compounds are also useful in treating any cancer driven by a mutated protein kinase, or any tumor driven by nuclear hormone receptors (e.g., androgen receptor (prostate), estrogen receptor (breast), progesterone receptor (breast)). Accordingly, in yet another aspect, according to the methods of treatment of the present invention, tumor cells are killed, or their growth is inhibited by contacting said tumor cells with an inventive compound or composition, as described herein.

In certain embodiments, the compounds described herein inhibit androgen signaling in prostate cancer cells and thereby lead to cell death. In certain embodiments, the compounds described herein inhibit estrogen or progesterone signaling in breast cancer cells and thereby lead to cell death. A therapeutically effective amount of the compound is administered to cells or a subject in order to inhibit receptor signaling. The inhibition of receptor signaling in these cells then leads to cell death. The method of inducing cell death is particularly useful in treating prostate and breast cancer.

Thus, in another aspect of the invention, methods for the treatment of cancer are provided comprising administering a therapeutically effective amount of an inventive compound, as described herein, to a subject in need thereof. In certain embodiments, a method for the treatment of cancer is provided comprising administering a therapeutically effective amount of an inventive compound, or a pharmaceutical composition comprising an inventive compound to a subject in need thereof, in such amounts and for such time as is necessary to achieve the desired result. In certain embodiments of the present invention a “therapeutically effective amount” of the inventive compound or pharmaceutical composition is that amount effective for killing or inhibiting the growth of tumor cells. The compounds and compositions, according to the method of the present invention, may be administered using any amount and any route of administration effective for killing or inhibiting the growth of tumor cells. Thus, the expression “amount effective to kill or inhibit the growth of tumor cells,” as used herein, refers to a sufficient amount of agent to kill or inhibit the growth of tumor cells. The exact amount required will vary from subject to subject, depending on the species, age, and general condition of the subject, the severity of the infection, the particular anticancer agent, its mode of administration, and the like.

In certain embodiments, the method involves the administration of a therapeutically effective amount of the compound or a pharmaceutically acceptable derivative thereof to a subject (including, but not limited to a human or animal) in need of it. In certain embodiments, the inventive compounds as useful for the treatment of cancer (including, but not limited to, glioblastoma, retinoblastoma, breast cancer, cervical cancer, colon and rectal cancer, leukemia (e.g., CML, AML, CLL, ALL), lymphoma, lung cancer (including, but not limited to small cell lung cancer), melanoma and/or skin cancer, multiple myeloma, non-Hodgkin's lymphoma, ovarian cancer, pancreatic cancer, prostate cancer, gastric cancer, bladder cancer, uterine cancer, kidney cancer, testicular cancer, stomach cancer, brain cancer, liver cancer, or esophageal cancer). In certain embodiments, the cancer is BCR/ABL chromic myeloid leukemia. In other embodiments, the cancer is an FLT3-mutant leukemia. In yet other embodiments, the cancer is an EGFR-mutant leukemia. In still other embodiments, the cancer is an AKT-mutant cancer. In certain embodiments, the cancer is driven by a mutated protein kinase. In other embodiments, the cancer is driven by a nuclear hormone receptor.

In certain embodiments, the inventive anticancer agents are useful in the treatment of cancers and other proliferative disorders, including, but not limited to breast cancer, cervical cancer, leukemia, lung cancer, ovarian cancer, and prostate cancer, to name a few. In certain embodiments, the inventive anticancer agents are active against prostate cancer cells. In certain embodiments, the inventive anticancer agents are active against leukemia cells. In other embodiments, the inventive anticancer agents are active against breast cancer cells. In still other embodiments, the inventive anticancer agents are active against lung cancer cells. In still other embodiments, the inventive anticancer agents are active against solid tumors.

In certain embodiments, the inventive compounds also find use in the prevention of restenosis of blood vessels subject to traumas such as angioplasty and stenting. For example, it is contemplated that the compounds of the invention will be useful as a coating for implanted medical devices, such as tubings, shunts, catheters, artificial implants, pins, electrical implants such as pacemakers, and especially for arterial or venous stents, including balloon-expandable stents. In certain embodiments inventive compounds may be bound to an implantable medical device, or alternatively, may be passively adsorbed to the surface of the implantable device. In certain other embodiments, the inventive compounds may be formulated to be contained within, or, adapted to release by a surgical or medical device or implant, such as, for example, stents, sutures, indwelling catheters, prosthesis, and the like. For example, drugs having antiproliferative and anti-inflammatory activities have been evaluated as stent coatings, and have shown promise in preventing retenosis (See, for example, Presbitero et al., “Drug eluting stents do they make the difference?”, Minerva Cardioangiol, 2002, 50(5):431-442; Ruygrok et al., “Rapamycin in cardiovascular medicine”, Intern. Med. J., 2003, 33(3):103-109; and Marx et al., “Bench to bedside: the development of rapamycin and its application to stent restenosis”, Circulation, 2001, 104(8):852-855, each of these references is incorporated herein by reference in its entirety). Accordingly, without wishing to be bound to any particular theory, Applicant proposes that inventive compounds having antiproliferative effects can be used as stent coatings and/or in stent drug delivery devices, inter alia for the prevention of restenosis or reduction of restenosis rate. Suitable coatings and the general preparation of coated implantable devices are described in U.S. Pat. Nos. 6,099,562; 5,886,026; and 5,304,121; each of which is incorporated herein by reference. The coatings are typically biocompatible polymeric materials such as a hydrogel polymer, polymethyldisiloxane, polycaprolactone, polyethylene glycol, polylactic acid, ethylene vinyl acetate, and mixtures thereof. The coatings may optionally be further covered by a suitable topcoat of fluorosilicone, polysaccarides, polyethylene glycol, phospholipids or combinations thereof to impart controlled release characteristics in the composition. A variety of compositions and methods related to stent coating and/or local stent drug delivery for preventing restenosis are known in the art (see, for example, U.S. Pat. Nos. 6,517,889; 6,273,913; 6,258,121; 6,251,136; 6,248,127; 6,231,600; 6,203,551; 6,153,252; 6,071,305; 5,891,507; 5,837,313 and published U.S. patent application: US2001/0027340, each of which is incorporated herein by reference in its entirety). For example, stents may be coated with polymer-drug conjugates by dipping the stent in polymer-drug solution or spraying the stent with such a solution. In certain embodiment, suitable materials for the implantable device include biocompatible and nontoxic materials, and may be chosen from the metals such as nickel-titanium alloys, steel, or biocompatible polymers, hydrogels, polyurethanes, polyethylenes, ethylenevinyl acetate copolymers, etc. In certain embodiments, the inventive compound is coated onto a stent for insertion into an artery or vein following balloon angioplasty.

The compounds of this invention or pharmaceutically acceptable compositions thereof may also be incorporated into compositions for coating implantable medical devices, such as prostheses, artificial valves, vascular grafts, stents and catheters. Accordingly, the present invention, in another aspect, includes a composition for coating an implantable device comprising a compound of the present invention as described generally above, and in classes and subclasses herein, and a carrier suitable for coating said implantable device. In still another aspect, the present invention includes an implantable device coated with a composition comprising a compound of the present invention as described generally above, and in classes and subclasses herein, and a carrier suitable for coating said implantable device.

Within other aspects of the present invention, methods are provided for expanding the lumen of a body passageway, comprising inserting a stent into the passageway, the stent having a generally tubular structure, the surface of the structure being coated with (or otherwise adapted to release) an inventive compound or composition, such that the passageway is expanded. In certain embodiments, the lumen of a body passageway is expanded in order to eliminate a biliary, gastrointestinal, esophageal, tracheal/bronchial, urethral and/or vascular obstruction.

Methods for eliminating biliary, gastrointestinal, esophageal, tracheal/bronchial, urethral and/or vascular obstructions using stents are known in the art. The skilled practitioner will know how to adapt these methods in practicing the present invention. For example, guidance can be found in U.S. Patent Publication No.: 2003/0004209 in paragraphs [0146]-[0155], which paragraphs are hereby incorporated herein by reference.

Another aspect of the invention relates to a method for inhibiting the growth of multidrug resistant cells in a biological sample or a patient, which method comprises administering to the patient, or contacting said biological sample with a compound of the invention or a composition comprising said compound.

Additionally, the present invention provides pharmaceutically acceptable derivatives of the inventive compounds, and methods of treating a subject using these compounds, pharmaceutical compositions thereof, or either of these in combination with one or more additional therapeutic agents.

Another aspect of the invention relates to a method of treating or lessening the severity of a disease or condition associated with a proliferation disorder in a patient, said method comprising a step of administering to said patient, a compound described herein or a composition comprising said compound.

It will be appreciated that the compounds and compositions, according to the method of the present invention, may be administered using any amount and any route of administration effective for the treatment of cancer and/or disorders associated with cell hyperproliferation. For example, when using the inventive compounds for the treatment of cancer, the expression “effective amount” as used herein, refers to a sufficient amount of agent to inhibit cell proliferation, or refers to a sufficient amount to reduce the effects of cancer. The exact amount required will vary from subject to subject, depending on the species, age, and general condition of the subject, the severity of the diseases, the particular anticancer agent, its mode of administration, and the like.

The compounds of the invention are preferably formulated in dosage unit form for ease of administration and uniformity of dosage. The expression “dosage unit form” as used herein refers to a physically discrete unit of therapeutic agent appropriate for the patient to be treated. It will be understood, however, that the total daily usage of the compounds and compositions of the present invention will be decided by the attending physician within the scope of sound medical judgment. The specific therapeutically effective dose level for any particular patient or organism will depend upon a variety of factors including the disorder being treated and the severity of the disorder; the activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts (see, for example, Goodman and Gilman's, “The Pharmacological Basis of Therapeutics”, Tenth Edition, A. Gilman, J. Hardman and L. Limbird, eds., McGraw-Hill Press, 155-173, 2001, which is incorporated herein by reference in its entirety).

Another aspect of the invention relates to a method for inhibiting Hsp90 activity in a biological sample or a patient, which method comprises administering to the patient, or contacting said biological sample with a compound described herein or a composition comprising said compound.

Furthermore, after formulation with an appropriate pharmaceutically acceptable carrier in a desired dosage, the pharmaceutical compositions of this invention can be administered to humans and other animals orally, rectally, parenterally, intracisternally, intravaginally, intraperitoneally, topically (as by powders, ointments, creams or drops), bucally, as an oral or nasal spray, or the like, depending on the severity of the infection being treated. In certain embodiments, the compounds of the invention may be administered at dosage levels of about 0.001 mg/kg to about 50 mg/kg, from about 0.01 mg/kg to about 25 mg/kg, or from about 0.1 mg/kg to about 10 mg/kg of subject body weight per day, one or more times a day, to obtain the desired therapeutic effect. It will also be appreciated that dosages smaller than 0.001 mg/kg or greater than 50 mg/kg (for example 50-100 mg/kg) can be administered to a subject. In certain embodiments, compounds are administered orally or parenterally.

### Treatment Kits

In other embodiments, the present invention relates to a kit for conveniently and effectively carrying out the methods in accordance with the present invention. In general, the pharmaceutical pack or kit comprises one or more containers filled with one or more of the ingredients of the pharmaceutical compositions of the invention. Such kits are especially suited for the delivery of solid oral forms such as tablets or capsules. Such a kit preferably includes a number of unit dosages, and may also include a card having the dosages oriented in the order of their intended use. If desired, a memory aid can be provided, for example in the form of numbers, letters, or other markings or with a calendar insert, designating the days in the treatment schedule in which the dosages can be administered. Alternatively, placebo dosages, or calcium dietary supplements, either in a form similar to or distinct from the dosages of the pharmaceutical compositions, can be included to provide a kit in which a dosage is taken every day. Optionally associated with such container(s) can be a notice in the form prescribed by a governmental agency regulating the manufacture, use or sale of pharmaceutical products, which notice reflects approval by the agency of manufacture, use or sale for human administration.

### EQUIVALENTS

The representative examples which follow are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples which follow and the references to the scientific and patent literature cited herein. It should further be appreciated that, unless otherwise indicated, the entire contents of each of the references cited herein are incorporated herein by reference to help illustrate the state of the art. The following examples contain important additional information, exemplification and guidance which can be adapted to the practice of this invention in its various embodiments and the equivalents thereof.

These and other aspects of the present invention will be further appreciated upon consideration of the following Examples, which are intended to illustrate certain particular embodiments of the invention but are not intended to limit its scope, as defined by the claims.

### EXAMPLES

### Example 1

### Gene Expression Signature-Based Chemical Genomic Prediction Identifies a Novel Class of HSP90 Pathway Modulators

### Introduction

Androgen receptor (AR)-mediated signaling represents a critical pathway in prostate cancer progression (Feldman et al., 2001). Hormonal therapies that reduce circulating androgen levels and inhibit the androgen receptor will initially block prostate cancer growth. Eventually, however, such therapies give rise to fatal drug-resistant, or hormone-refractory, disease. Hormone-refractory prostate cancers commonly show reactivation of AR-mediated signaling through a number of mechanisms (Chen et al., 2004, Feldman et al., 2001, Linja et al., 2001). Androgen-independent tumors often show expression of AR and of AR-induced genes such as PSA. Approximately one- to two-fifths of androgen-independent tumors exhibit increased AR expression after androgen ablation (Linja et al., 2001, Visakorpi et al., 1995), and such AR overexpression appears to allow prostate cancer growth in the face of decreased androgen levels (Chen et al., 2004). Critically, overall expression patterns of androgen-independent tumors are more similar to those of untreated androgen-dependent primary cancers than to those of tumors after neoadjuvant androgen deprivation, suggesting reactivation of AR-mediated transcription (Holzbeierlein et al., 2004).

Though androgen signaling is critical to prostate cancer progression, our ability to modulate AR-mediated signaling programs is limited. Secondary hormonal therapies beyond androgen ablation primarily target ligand-mediated activation of AR, but none appear to be permanently effective against AR signaling-mediated cancer progression (Lam et al., 2006). Additional therapies are in development that may target both AR-mediated signaling and cooperative signaling pathways. Heat shock protein 90 (HSP90) inhibitors, for example, suppress AR signaling and other fundamental oncogenic pathways by promoting degradation of hormone receptors, kinases, and other client proteins (Whitesell et al., 2005). In general, the current lack of effective AR signaling inhibitors highlights the need for modulators of AR signaling across the full spectrum of AR biology.

Discovery of compounds that modulate complex cancer phenotypes such as androgen independence and signaling represents a challenging problem in chemical biology. Gene expression-based chemical discovery has the potential to identify compounds that convert one biological state, as defined by its gene expression signature, to that of a more desirable state without first assaying or identifying each critical effector in the process (Stegmaier et al., 2004). In cancer biology, gene expression-based screening (GE-HTS) allows identification of compounds that revert undesired oncogenic states to those of more nonmalignant or drug-sensitive states. Broadly, gene expression-based chemical discovery represents a strategy for identifying modulators of biological processes with little a priori information about their underlying mechanisms.

An additional problem in chemical biology, perhaps more significant than chemical discovery itself, is the identification of compounds' targets following cell-based discovery (di Bernardo et al., 2005, Gardner et al., 2003). Recent work has applied unbiased gene expression-based approaches to prediction of chemical activity and targets in bacteria and yeast (di Bernardo et al., 2005, Gardner et al., 2003, Parsons et al., 2004). Nonetheless, chemical genomic prediction has not been applied to complex mammalian systems.

Here we illustrate a robust, generalizable approach for chemical genomic discovery and prediction in mammalian cells. Given the limited means available to identify modulators of critical AR signaling pathways and their mechanisms, we set out to discover AR signaling inhibitors using a gene expression signature-based screening approach. Of the hits that emerged, celastrol and gedunin compounds represent a structurally similar group of natural products with a history of medicinal and anticancer use. To investigate the target activity of these compounds, we used an approach to connect the activities of celastrol and gedunin to drugs with known biological activities at the gene expression level, using a compendium of gene expression profiles of drug treatment. Celastrol and gedunin both invoked gene expression signatures highly similar to those of existing HSP90 inhibitors. Subsequent work validated this gene expression-based activity prediction. However, celastrol and gedunin do not act directly on the HSP90 ATP-binding pocket, unlike most existing HSP90 inhibitors. Instead, they act synergistically with existing HSP90 inhibitors to suppress HSP90 client signaling and viability. In all, we demonstrate the discovery of HSP90 functional inhibition through a generalizable gene expression-based approach for compound discovery and elucidation.

### Results

### Gene Expression-Based Screen Identifies Inhibitors of Androgen Receptor (AR) Activation Signature

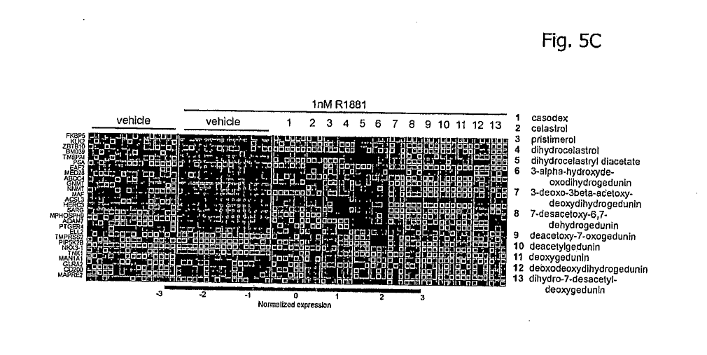

Because of the paucity of effective AR-mediated signaling inhibitors, we set out to identify new inhibitors of AR activation using a gene expression signature-based screening approach (Stegmaier et al., 2004). GE-HTS identifies compounds that convert a gene expression signature representing one state to that of another, using a high-throughput bead-based method to quantify the gene expression signatures (FIG. 5A; Peck et al., 2006). Here, we asked whether GE-HTS could be used to identify androgen signaling modulators that revert the signature of the androgen-activated state to the signature of the quiescent, androgen-deprived state in prostate cancer cells.

Toward that end, we first defined the gene expression signature of AR activation in the LNCaP prostate cancer cell line, a common in vitro model of AR-mediated signaling in prostate cancer (Chen et al., 2004). The signature was defined by identifying genes that are activated or repressed by androgen stimulation (0.1 nM R1881, 24 hr) relative to androgen deprivation, using microarray-based gene expression profiling (Febbo et al., 2005). The AR activation signature was refined to 27 genes that showed robust activation or inhibition of expression upon androgen stimulation as measured in our GE-HTS bead-based assay (FIG. 5B). The final 27 gene signature therefore represents a gene set that associates with androgen signaling at a selected level of robustness.

Next, we asked whether the multigene GE-HTS approach provides significant advantages over conventional screening approaches for androgen signaling inhibitors. We found that the GE-HTS method performed better than a single reporter assay due to the robustness provided by a multigene readout. Compared to a single-gene readout using the best marker gene in the microarray data, the 27 gene signature decreased the false-positive rate of our screen 14-fold and the false-negative rate 7-fold, as determined by leave-one-out cross-validation using weighted voting and K-nearest neighbors analysis. Further, GE-HTS allows the assay of endogenous AR-mediated gene induction and repression, rather than expression in a non-chromatin reporter system.

GE-HTS screening was then carried out for compounds that convert the AR activation signature to the androgen-deprived signature. Compound libraries comprising approximately 2500 compounds and enriched in drugs and natural products were screened. LNCaP cells were treated for 24 hr with synthetic androgen R1881 and compound for the GE-HTS screen. In parallel, the libraries were screened for their effects on LNCaP viability over 3 days using a luminescent ATP quantitation assay.

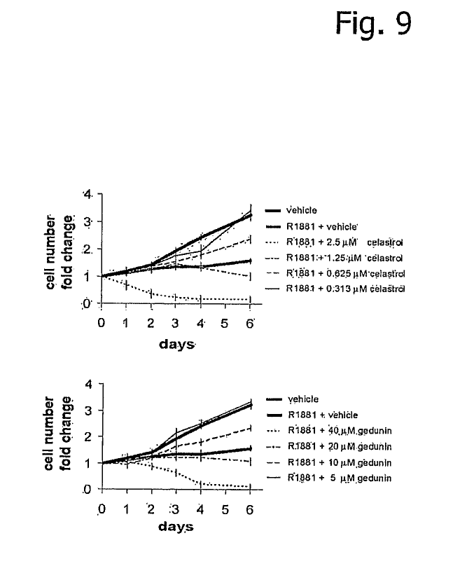

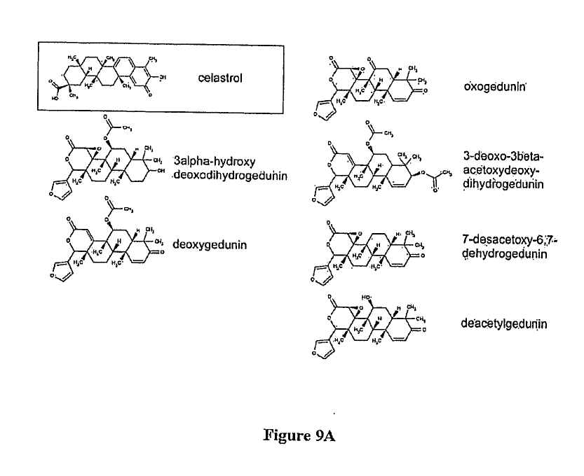

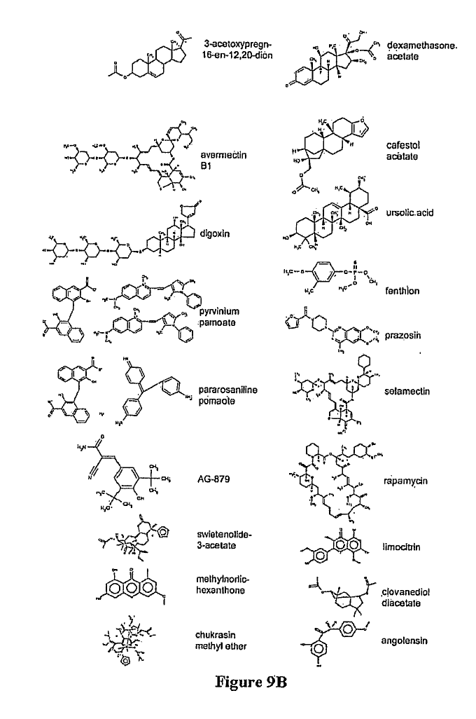

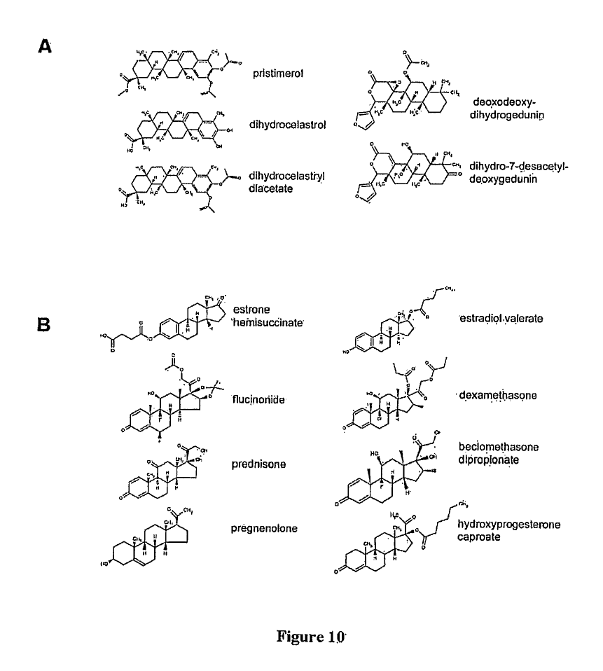

The screen identified more than 20 compounds that robustly suppress the androgen signaling signature without causing severe toxicity in vitro, while another 30 were found to mildly inhibit the signature (FIGS. 9 and 10; Table 1). Compounds that inhibit the androgen signaling signature were identified using three analytic metrics: summed gene expression, K-nearest neighbors, and naive Bayes classification. These metrics incorporate both supervised and unsupervised approaches as well as parametric and nonparametric statistics. Strong hits were defined as compounds that induced the androgen deprivation signature in at least two of three replicates by all three measures at p<0.05. Weak hits were defined as compounds that induced the androgen deprivation signature in at least two of three replicates by only two measures (p<0.05). These hits were subsequently filtered to remove compounds that inhibited cell growth by more than 50% over 3 days.

TABLE 1
GE-HTS screen hits.

| | GE-HTS screen # replicates that score as the androgon deprived state (n = 3) | | | | | viability screen | |
|---|---|---|---|---|---|---|---|
| | unweighted | | | | | viability (% [ATP] | |
| | summed | weighted | | Naïve Bayes | | normalized to | |
| | score | summed score | | classifier | screen day 1 | R1881-treated | c.v. of |
| Name | (P < 0.05) | (P < 0.05) | KNN | (P < 0.05) | or day 2 | control) | viability |

i. Controls

| | | | | | | | |
|---|---|---|---|---|---|---|---|
| androgen treated (n = 1986) | | | 1950 | | 0 and 2 | 100.0 | 10.2 |
| androgen deprivation (n = 1986) | | | 1959 | | 1 and 2 | 63.2 | 7.8 |
| casodex (n = 336) | 257 | 261 | 283 | 268 | 1 and 2 | | |

ii. Strong hits (stringent)

| | | | | | | | |
|---|---|---|---|---|---|---|---|
| digoxin | 3 | 3 | 3 | 3 | 1 | 104.5 | 13.5 |
| prazosin | 3 | 3 | 3 | 3 | 1 | 59.5 | 4.5 |
| celastrol | 3 | 3 | 3 | 3 | 1 | 51.6 | 16.0 |
| pyrvinium pamoate | 3 | 3 | 3 | 3 | 1 | 50.4 | 21.3 |
| pararosaniline pamoate | 3 | 3 | 3 | 3 | 1 | 52.5 | 13.9 |
| deacetoxy-7-oxogedunin | 3 | 3 | 3 | 3 | 2 | 69.0 | 5.2 |
| ursolic acid | 3 | 3 | 3 | 3 | 2 | 80.5 | 6.7 |
| 3-deoxo-3beta- | | | | | | | |
| acetoxydeoxydihydrogedunin | 3 | 3 | 3 | 3 | 2 | 51.0 | 6.9 |
| 7-desacetoxy-6,7-dehydrogedunin | 3 | 3 | 3 | 3 | 2 | 63.1 | 8.7 |
| fenthion | 3 | 3 | 3 | 3 | 2 | 50.5 | 9.2 |
| cafestol acetate | 3 | 3 | 3 | 3 | 2 | 60.8 | 9.5 |
| chukrasin methyl ether | 3 | 3 | 3 | 3 | 2 | 60.1 | 13.2 |
| angolensin | 3 | 3 | 3 | 3 | 2 | 57.7 | 13.4 |
| 3alpha- | | | | | | | |
| hydroxydeoxodihydrogedunin | 3 | 3 | 3 | 3 | 2 | 55.9 | 14.9 |
| clovanediol diacetate | 3 | 3 | 3 | 3 | 2 | 87.4 | 15.2 |
| selamectin | 2 | 2 | 3 | 3 | 1 | 81.1 | 12.2 |
| dexamethasone acetate | 2 | 2 | 3 | 2 | 1 | 90.1 | 31.7 |
| avermectin B1 | 2 | 2 | 3 | 2 | 1 | 53.7 | 3.5 |
| limocitrin | 3 | 3 | 2 | 3 | 2 | 63.2 | 18.3 |
| deoxygedunin | 3 | 3 | 2 | 2 | 2 | 63.3 | 6.7 |
| rapamycin | 2 | 3 | 3 | 3 | 2 | 59.4 | 0.4 |
| 3-acetoxypregn-16-en-12,20-dione | 2 | 3 | 3 | 3 | 2 | 65.3 | 6.2 |
| methylnortichexanthone | 2 | 2 | 3 | 2 | 2 | 60.1 | 4.0 |
| deacetylgedunin | 2 | 2 | 3 | 2 | 2 | 67.8 | 9.8 |
| AG-879 | 2 | 2 | 3 | 2 | 2 | 54.6 | 20.6 |
| swietenolide-3-acetate | 2 | 2 | 2 | 2 | 2 | 72.2 | 2.7 |

iii. Weak hits (relaxed)

| | | | | | | | |
|---|---|---|---|---|---|---|---|
| butacaine | 2 | 2 | 0 | 2 | 1 | 77.4 | 44.1 |
| terfenadine | 1 | 1 | 2 | 2 | 1 | 86.9 | 1.8 |
| trifluoperazine | 0 | 2 | 3 | 3 | 1 | 82.6 | 33.5 |
| estrone hemisuccinate | 0 | 1 | 3 | 3 | 1 | 71.9 | 7.6 |
| fluocinonide | 0 | 1 | 2 | 2 | 1 | 76.6 | 15.7 |
| cetrimonium bromide | 0 | 0 | 3 | 3 | 1 | 109.7 | 3.9 |
| dimenhydrinate | 0 | 0 | 3 | 3 | 1 | 102.9 | 8.1 |
| pregnenolone | 0 | 0 | 3 | 2 | 1 | 91.5 | 5.1 |
| estradiol valerate | 0 | 0 | 3 | 3 | 1 | 84.5 | 14.3 |
| pramoxine | 0 | 0 | 3 | 3 | 1 | 82.1 | 8.3 |
| dexamethasone | 0 | 0 | 3 | 3 | 1 | 73.2 | 26.2 |
| hydroxyprogesterone caproate | 0 | 0 | 3 | 2 | 1 | 65.7 | 5.5 |
| beclomethasone dipropionate | 0 | 0 | 3 | 2 | 1 | 64.8 | 13.0 |
| monensin A | 0 | 0 | 2 | 2 | 1 | 113.6 | 22.8 |
| nystatin | 0 | 0 | 2 | 2 | 1 | 74.5 | 26.1 |
| exalamide | 0 | 0 | 2 | 2 | 1 | 73.9 | 20.3 |
| prednisone | 0 | 0 | 2 | 2 | 1 | 66.2 | 18.3 |
| abienol | 3 | 3 | 2 | 1 | 2 | 64.1 | 6.7 |
| 11-oxoursolic acid acetate | 3 | 2 | 3 | 1 | 2 | 83.5 | 6.7 |
| pristimerol | 3 | 1 | 3 | 1 | 2 | 50.2 | 17.4 |
| methyl parathione | 3 | 0 | 3 | 1 | 2 | 65.8 | 14.2 |
| deoxodeoxydihydrogedunin | 2 | 2 | 0 | 3 | 2 | 51.8 | 12.2 |
| dihydro-7-desacetyldeoxygedunin | 2 | 1 | 3 | 2 | 2 | 75.6 | 3.5 |
| isoliquiritigenin | 2 | 1 | 2 | 1 | 2 | 87.0 | 2.6 |
| harmol hydrochloride | 2 | 0 | 3 | 0 | 2 | 71.6 | 24.2 |
| dihydrocelastrol | 2 | 0 | 3 | 0 | 2 | 53.1 | 14.1 |
| acacetin diacetate | 2 | 0 | 2 | 0 | 2 | 112.6 | 10.9 |
| atovaquone | 1 | 1 | 3 | 2 | 2 | 76.6 | 8.9 |
| dihydrocelastryl diacetate | 3 | 0 | 2 | 0 | 2 | 50.7 | 9.9 |

This file lists all hits found in the GE-HTS screens of NINDS, SpecPlus, and BioMol libraries for androgen signaling signature inhibitors.

Many of the identified androgen signaling signature inhibitors have provocative activities. They include prazosin, a drug currently used for treatment of benign prostatic hyperplasia (Walsh, 1996), and the mTOR inhibitor rapamycin, which is currently in clinical trials as a treatment for advanced prostate cancer (Majumder et al., 2005). Dexamethasone acetate was also found to strongly inhibit the androgen signaling signature, and a range of other glucocorticoids were identified as weak inhibitors; glucocorticoids are currently used for their systemic effects in prostate cancer treatment but may also have a direct effect on prostate cancer cell signaling (Lam et al., 2006). Most notably, a large set of celastrol and gedunin natural products made up more than a quarter of the identified AR signaling inhibitors (FIG. 5C), and these compounds were therefore studied in greater detail.

Celastrol, Gedunin, and Derivatives Represent a Structurally Related Group of Natural Products that Inhibit Androgen Signaling

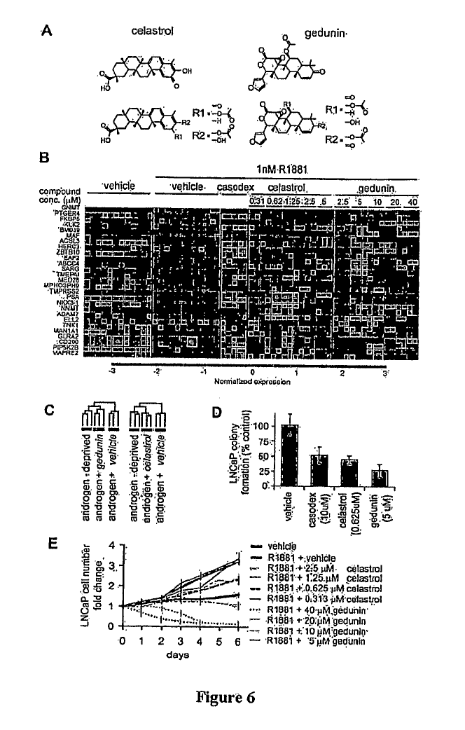

The celastrol and gedunin triterpenoids represent a dominant family of structurally similar compounds that emerged from our GE-HTS screen (FIGS. 5C and 6A). Celastrol and six gedunin derivatives showed strong inhibition of the androgen signaling signature (FIG. 5C), while two gedunin derivatives and three celastrol derivatives also showed weak inhibitory activity (Table 1). Celastrol and gedunin are natural products derived from plants of the Celastracae and Meliacae families that have been used therapeutically for several millennia, though little is known about their cellular targets (Padma, 2005, Ushiro et al., 1997). Celastrol and gedunin compounds show structural similarity (FIG. 6A; FIGS. 9A and 10A). Moreover, celastrol and gedunin invoked similar global gene expression changes, when we assayed the gene expression effects of celastrol (1.25 μM, 6 hr) and gedunin (20 μM, 6 hr) by genome-wide DNA microarray. The genes regulated by celastrol and gedunin were highly overlapping (p<10⁻¹⁸, Fisher's exact test). Celastrol, gedunin, and their derivatives therefore represent a family of AR signaling inhibitors with similar structure and activity at the gene expression level.

To validate the effect of celastrol and gedunin on AR-mediated signaling, we first established that they inhibit the GE-HTS androgen signaling signature in a concentration-dependent manner in LNCaP cells (FIG. 6B). Because natural products often contain impurities, we verified that celastrol and gedunin used for this work were >98% and >99% pure, respectively, by HPLC and NMR. Celastrol- and gedunin-induced inhibition was seen both with and without 12 hours pretreatment with androgen (FIG. 6B). Celastrol and gedunin therefore inhibit the androgen signaling signature outside the screen context.

We next asked whether celastrol and gedunin inhibit the broader program of androgen signaling beyond the GE-HTS signature. To address this question, we compared the genome-wide gene expression profiles of androgen-stimulated LNCaP cells treated with celastrol (1.25 μM) and gedunin (20 μM) for 24 hr to those of androgen-stimulated and androgen-deprived cells. Hierarchical clustering indicated that androgen-responsive gene expression (Febbo et al., 2005) of compound-treated androgen-stimulated cells is more similar to that of androgen-deprived cells than to that of vehicle-treated androgen-stimulated cells (FIG. 6C). Celastrol and gedunin treatment therefore invoked a broader gene expression program similar to that induced by androgen deprivation, though differences between them can still be seen.

To investigate the cellular consequences of celastrol- and gedunin-mediated inhibition, we assessed whether celastrol and gedunin activity results in decreased cell growth, consistent with AR inhibition. First, we determined whether the compounds inhibit adherent growth of androgen-stimulated LNCaP cells by luminescent assay of ATP levels. The compounds mimic the growth-inhibitory effects of androgen deprivation around the EC₅₀ of androgen signaling inhibition (FIGS. 6D and 6E). Second, the compounds' effects on anchorage-independent growth of LNCaP cells was assayed in soft agar (FIG. 6D). Celastrol (0.625 μM) and gedunin inhibited anchorage-independent growth to a similar degree as the AR competitive antagonist bicalutamide (casodex). In addition to reducing colony number, celastrol and gedunin inhibited colony size. Celastrol and gedunin therefore inhibit adherent and anchorage-independent growth of LNCaP cells, likely, in part, due to suppression of AR signaling.

### Gene Expression Compendium of Drug Effects Identifies Hsp90-Inhibitory Activity of Celastrol and Gedunin

While celastrol and gedunin clearly inhibit AR-mediated signaling, their target and mechanism are not obvious. Indeed, a major challenge in cell-based chemical biology and chemical genomics is the identification of compounds' targets (Gardner et al., 2003). We hypothesized that gene expression signatures could be used to identify compound action based on the similarity of such compound-induced signatures to signatures of existing drugs of known mechanism. We therefore employed a collection of gene expression profiles of drug-treated cell lines that was developed in our lab, termed the Connectivity Map (Lamb et al., 2006). This database comprises 453 genome-wide Affymetrix expression profiles derived from the treatment of human cell lines with 164 small molecules, primarily FDA-approved drugs. A 6 hr treatment time was chosen in an attempt to capture the primary, and potentially mechanistic, effects of the compounds rather than the downstream phenotypic consequences.

In order to use the Connectivity Map to gain insight into celastrol and gedunin function, we first defined a gene expression signature of celastrol and gedunin activity. The expression signatures of celastrol and gedunin were derived by expression profiling of RNA from LNCaP cells treated with celastrol (1.25 μM), gedunin (20 μM), and vehicle (DMSO) for 6 hr; signatures were defined using comparative marker selection to identify transcripts that distinguished between the compound- and vehicle-treated profiles by the signal-to-noise (SNR) metric. The enrichment of these signatures in the gene expression profiles of the Connectivity Map database was then assessed using a gene enrichment metric, the connectivity score, based on the Kolmogorov-Smirnov statistic (Lamb et al., 2003). Out of 164 compounds represented by the Connectivity Map, celastrol was the top match for the gedunin signature and the fourth-ranked match for the celastrol signature (Table 2). The enrichment of the LNCaP celastrol signature in the MCF7 celastrol gene expression profile validates our ability to identify true similarities using the Connectivity Map and their cell line independence. Moreover, the enrichment of the gedunin signature in the celastrol profile demonstrates similarity between celastrol and gedunin activities.

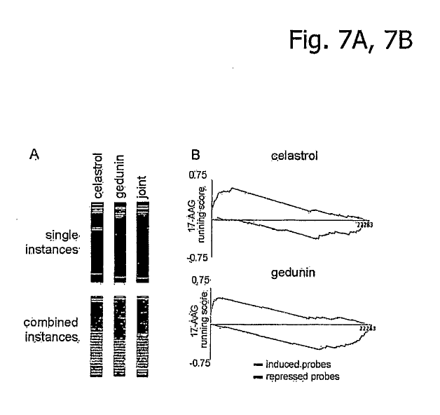

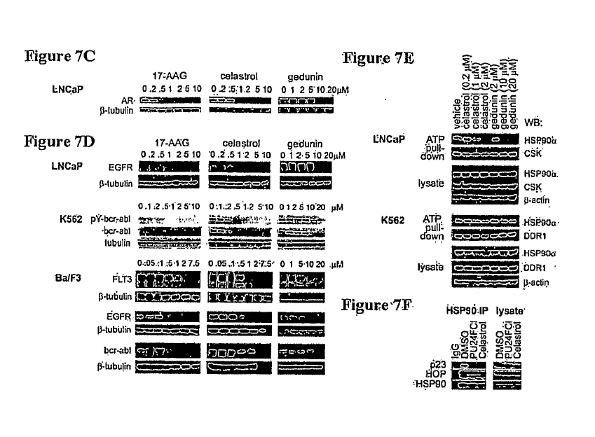

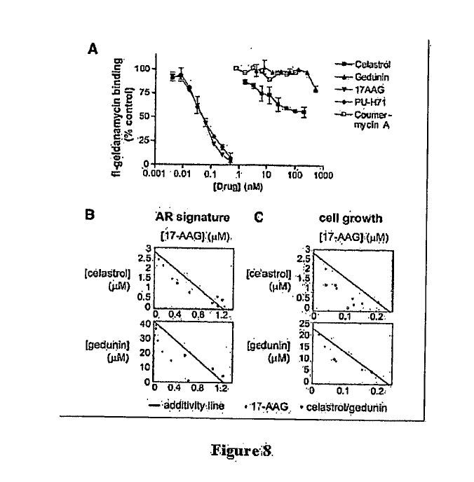

To generate hypotheses regarding celastrol and gedunin targets, the Connectivity Map was used to identify known drugs with highly similar gene expression effects. The celastrol and gedunin signatures showed very strong similarity to the gene expression profiles of four HSP90 inhibitors: geldanamycin (n=6), 17-dimethylaminoethylamino-17-demethoxy-geldanamycin (17-DMAG; n=2), 7-allylamino-17-demethoxygeldanamycin (17-AAG; n=18), and monorden (radicicol; n=10) (FIG. 7A; Table 2). Geldanamycin and its derivatives induced gene expression profiles that were highly enriched with celastrol and gedunin signature genes at 6 hr, as shown by the high enrichment score ranking of these compounds relative to other compounds in the Connectivity Map database (FIG. 7A). For example, celastrol- and gedunin-induced genes were enriched in the 17-AAG profile (FIG. 7B, green), whereas celastrol- and gedunin-repressed genes were similarly repressed by 17-AAG (FIG. 7B, red). In contrast, a signature of randomly selected genes did not show enrichment over this 17-AAG profile (FIG. 7B). The radicicol profiles were similarly enriched, albeit to a lesser extent. The similarity of celastrol and gedunin activities to HSP90 inhibition is supported by the significant number of replicates (FIG. 7A, single instances) and the number of different HSP90 inhibitors (FIG. 7A, combined instances) that show this enrichment. The signatures of 24 hours celastrol and gedunin treatment also showed similarity to the HSP90 inhibitor profiles, though to a somewhat lesser degree. HSP90 inhibition therefore represents a major gene expression signature invoked by celastrol and gedunin. More generally, this work illustrates a robust approach for using gene expression signatures to gain insight into chemical activity.

TABLE 2
Connectivity map query results.

| | | | | | |
|---|---|---|---|---|---|
| | # | gedunin | gedunin | celastrol | celastrol |
| name | instances | score | rank | score | rank |

i. Connectivity map compounds with multiple instances (replicates)

| | | | | | | |
|---|---|---|---|---|---|---|
| TAM-4 | | 2 | 0.911 | 3 | 0.877 | 3 |
| geldanamycin | HSP90 inhibitor | 6 | 0.74 | 4 | 0.676 | 11 |
| 17-dimethylamino-geldanamycin | HSP90 inhibitor | 2 | 0.73 | 5 | 0.766 | 6 |
| 17-allylamino-geldanamycin | HSP90 inhibitor | 18 | 0.702 | 6 | 0.736 | 8 |
| calmidazolium | | 2 | 0.619 | 7 | 0.334 | 42 |
| 15-delta prostaglandin J2 | | 5 | 0.565 | 9 | 0.76 | 7 |
| oxaprozin | | 2 | 0.562 | 10 | 0.445 | 30 |
| trifluoperazine | | 3 | 0.559 | 11 | 0.438 | 31 |
| vorinostat | | 2 | 0.554 | 13 | 0 | 122 |
| thioridazine | | 4 | 0.522 | 16 | 0.308 | 46 |
| monorden | HSP90 inhibitor | 10 | 0.511 | 17 | 0.409 | 36 |
| prochlorperazine | | 3 | 0.507 | 19 | 0.559 | 15 |
| docosahexaenoic acid ethyl ester | | 2 | 0.493 | 21 | 0.572 | 14 |
| pyrvinium | | 2 | 0.476 | 23 | 0.518 | 22 |
| butein | | 2 | 0.45 | 27 | 0.295 | 47 |
| trichostatin A | | 12 | 0.427 | 29 | −0.02 | 141 |
| carbamazepine | | 3 | 0.407 | 30 | 0.339 | 41 |
| resveratrol | | 5 | 0.4 | 31 | 0.266 | 49 |
| LM-1685 | | 3 | 0.371 | 34 | 0.118 | 67 |
| 5253409 | | 2 | 0.365 | 35 | 0.801 | 5 |
| fluphenazine | | 4 | 0.34 | 40 | 0.481 | 25 |
| 5248896 | | 2 | 0.312 | 41 | 0.445 | 29 |
| 5255229 | | 2 | 0.307 | 42 | 0 | 83 |
| raloxifene | | 3 | 0.292 | 44 | 0 | 85 |
| nordihydroguaiaretic acid | | 5 | 0.286 | 45 | 0.264 | 50 |
| W-13 | | 2 | 0.283 | 46 | 0 | 117 |
| 5211181 | | 2 | 0.273 | 47 | 0.521 | 21 |
| 5182598 | | 2 | 0.266 | 48 | 0.966 | 1 |
| novobiocin | HSP90 inhibitor | 6 | 0.261 | 49 | 0.167 | 61 |
| chlorpromazine | | 4 | 0.218 | 50 | 0.08 | 74 |
| tamoxifen | | 3 | 0.216 | 51 | 0 | 130 |
| mercaptopurine | | 2 | 0.21 | 52 | −0.121 | 150 |
| rottlerin | | 3 | 0.209 | 53 | 0.355 | 40 |
| colchicine | | 2 | 0.192 | 54 | 0.391 | 37 |
| 5252917 | | 2 | 0.192 | 55 | 0.582 | 12 |
| fulvestrant | | 7 | 0.19 | 56 | 0.069 | 75 |
| arachidonyltrifluoromethane | | 2 | 0.185 | 57 | 0.239 | 51 |
| sulindac | | 2 | 0.177 | 58 | 0.207 | 57 |
| depudecin | | 2 | 0.176 | 59 | 0.22 | 56 |
| haloperidol | | 6 | 0.172 | 60 | 0.131 | 66 |
| felodipine | | 3 | 0.171 | 61 | 0.171 | 60 |
| troglitazone | | 6 | 0.167 | 62 | 0.42 | 34 |
| nifedipine | | 2 | 0.149 | 63 | 0.153 | 65 |
| rofecoxib | | 6 | 0.148 | 64 | 0.088 | 72 |
| valproic acid | | 18 | 0.136 | 65 | 0.086 | 73 |
| ionomycin | | 3 | 0.129 | 66 | 0.556 | 17 |
| wortmannin | | 8 | 0.126 | 67 | −0.324 | 155 |
| thalidomide | | 2 | 0.121 | 68 | −0.256 | 153 |
| ciclosporin | | 2 | 0.121 | 69 | 0.414 | 35 |
| celecoxib | | 5 | 0.114 | 70 | −0.01 | 140 |
| iloprost | | 3 | 0.101 | 71 | 0.098 | 70 |
| cobalt chloride | | 3 | 0.1 | 72 | 0.269 | 48 |
| genistein | | 7 | 0.097 | 73 | 0.153 | 64 |
| TTNPB | | 2 | 0.095 | 74 | 0 | 97 |
| SC-58125 | | 4 | 0.09 | 75 | −0.112 | 149 |
| indometacin | | 4 | 0.087 | 76 | 0.228 | 53 |
| monastrol | | 8 | 0.069 | 77 | 0.226 | 55 |
| copper sulfate | | 4 | 0.066 | 78 | 0.113 | 68 |
| pirinixic acid | | 5 | 0.055 | 79 | 0.226 | 54 |
| alpha-estradiol | | 6 | 0.045 | 80 | −0.066 | 145 |
| tacrolimus | | 3 | 0 | 81 | 0 | 110 |
| rosiglitazone | | 4 | 0 | 86 | 0.098 | 69 |
| colforsin | | 2 | 0 | 90 | 0 | 129 |
| deferoxamine | | 3 | 0 | 95 | −0.175 | 151 |
| 5279552 | | 2 | 0 | 98 | 0.556 | 18 |
| 4,5-dianilinophthalimide | | 2 | 0 | 102 | 0 | 89 |
| LY-294002 | | 17 | 0 | 103 | −0.512 | 158 |
| blebbistatin | | 2 | 0 | 107 | 0 | 87 |
| fludrocortisone | | 2 | 0 | 108 | 0 | 98 |
| clofibrate | | 2 | 0 | 109 | 0 | 93 |
| sirolimus | | 10 | 0 | 113 | −0.283 | 154 |
| fasudil | | 2 | 0 | 115 | 0 | 100 |
| dexamethasone | | 3 | 0 | 123 | 0 | 108 |
| ikarugamycin | | 3 | 0 | 124 | 0.172 | 59 |
| imatinib | | 2 | 0 | 126 | 0.163 | 62 |
| chlorpropamide | | 2 | 0 | 128 | 0 | 111 |
| staurosporine | | 4 | 0 | 138 | 0.092 | 71 |
| acetylsalicylic acid | | 3 | 0 | 139 | 0.154 | 63 |
| sodium phenylbutyrate | | 7 | −0.029 | 144 | −0.083 | 146 |
| tetraethylenepentamine | | 6 | −0.092 | 145 | −0.039 | 144 |
| metformin | | 5 | −0.108 | 146 | −0.029 | 142 |
| bucladesine | | 3 | −0.144 | 147 | 0 | 123 |
| diclofenac | | 2 | −0.165 | 148 | −0.197 | 152 |
| estradiol | | 10 | −0.166 | 149 | 0.017 | 76 |
| tretinoin | | 8 | −0.206 | 150 | −0.084 | 147 |
| arachidonic acid | | 3 | −0.222 | 151 | 0 | 120 |
| NU-1025 | | 2 | −0.222 | 152 | −0.362 | 156 |
| exisulind | | 2 | −0.277 | 153 | −0.038 | 143 |
| clozapine | | 2 | −0.291 | 154 | 0 | 118 |
| MK-886 | | 2 | −0.336 | 155 | 0 | 94 |
| Y-27632 | | 2 | −0.353 | 156 | 0 | 107 |
| prazosin | | 2 | −0.358 | 157 | 0.231 | 52 |
| 5230742 | | 2 | −0.401 | 158 | 0.203 | 58 |
| quercetin | | 2 | −0.431 | 159 | −0.111 | 148 |
| benserazide | | 2 | −0.441 | 160 | 0 | 78 |

ii. All Connectivity Map compounds

| | | | | | | |
|---|---|---|---|---|---|---|
| celastrol | | 1 | 0.997 | 1 | 0.85 | 4 |
| MG-132 | | 1 | 0.912 | 2 | 0.902 | 2 |
| TAM-4 | | 2 | 0.911 | 3 | 0.877 | 3 |
| geldanamycin | HSP90 inhibitor | 6 | 0.74 | 4 | 0.676 | 11 |
| 17-dimethylamino-geldanamycin | HSP90 inhibitor | 2 | 0.73 | 5 | 0.766 | 6 |
| 17-allylamino-geldanamycin | HSP90 inhibitor | 18 | 0.702 | 6 | 0.736 | 8 |
| calmidazolium | | 2 | 0.619 | 7 | 0.334 | 42 |
| clotrimazole | | 1 | 0.577 | 8 | 0.557 | 16 |
| 15-delta prostaglandin J2 | | 5 | 0.565 | 9 | 0.76 | 7 |
| oxaprozin | | 2 | 0.562 | 10 | 0.445 | 30 |
| trifluoperazine | | 3 | 0.559 | 11 | 0.438 | 3.1 |
| BW-B70C | | 1 | 0.557 | 12 | 0.719 | 10 |
| vorinostat | | 2 | 0.554 | 13 | 0 | 122 |
| tyrphostin AG-1478 | | 1 | 0.529 | 14 | 0 | 127 |
| HC toxin | | 1 | 0.526 | 15 | 0 | 90 |
| thioridazine | | 4 | 0.522 | 16 | 0.308 | 46 |
| monorden | HSP90 inhibitor | 10 | 0.511 | 17 | 0.409 | 36 |
| 5666823 | | 1 | 0.509 | 18 | 0.446 | 28 |
| prochlorperazine | | 3 | 0.507 | 19 | 0.559 | 15 |
| pararosaniline | | 1 | 0.496 | 20 | 0.579 | 13 |
| docosahexaenoic acid ethyl ester | | 2 | 0.493 | 21 | 0.572 | 14 |
| cytochalasin B | | 1 | 0.476 | 22 | 0 | 125 |
| pyrvinium | | 2 | 0.476 | 23 | 0.518 | 22 |
| (−)-catechin | | 1 | 0.457 | 24 | 0 | 113 |
| pentamidine | | 1 | 0.456 | 25 | 0 | 101 |
| 5286656 | | 1 | 0.456 | 26 | 0.725 | 9 |
| butein | | 2 | 0.45 | 27 | 0.295 | 47 |
| nocodazole | | 1 | 0.436 | 28 | 0.517 | 23 |
| trichostatin A | | 12 | 0.427 | 29 | −0.02 | 141 |
| carbamazepine | | 3 | 0.407 | 30 | 0.339 | 41 |
| resveratrol | | 5 | 0.4 | 31 | 0.266 | 49 |
| 5162773 | | 1 | 0.387 | 32 | 0.532 | 20 |
| 2-deoxy-D-glucose | | 1 | 0.378 | 33 | 0 | 116 |
| LM-1685 | | 3 | 0.371 | 34 | 0.118 | 67. |
| 5253409 | | 2 | 0.365 | 35 | 0.801 | 5 |
| HNMPA-(AM)3 | | 1 | 0.365 | 36 | 0 | 103 |
| azathioprine | | 1 | 0.361 | 37 | 0.552 | 19 |
| 5140203 | | 1 | 0.354 | 38 | 0 | 114 |
| 5213008 | | 1 | 0.348 | 39 | 0.472 | 26 |
| fluphenazine | | 4 | 0.34 | 40 | 0.481 | 25 |
| 5248896 | | 2 | 0.312 | 41 | 0.445 | 29 |
| 5255229 | | 2 | 0.307 | 42 | 0 | 83 |
| oligomycin | | 1 | 0.305 | 43 | −0.491 | 157 |
| raloxifene | | 3 | 0.292 | 44 | 0 | 85 |
| nordihydroguaiaretic acid | | 5 | 0.286 | 45 | 0.264 | 50 |
| W-13 | | 2 | 0.283 | 46 | 0 | 117 |
| 5211181 | | 2 | 0.273 | 47 | 0.521 | 21 |
| 5182598 | | 2 | 0.266 | 48 | 0.966 | 1 |
| novobiocin | HSP90 inhibitor | 6 | 0.261 | 49 | 0.167 | 61 |
| chlorpromazine | | 4 | 0.218 | 50 | 0.08 | 74 |
| tamoxifen | | 3 | 0.216 | 51 | 0 | 130 |
| mercaptopurine | | 2 | 0.21 | 52 | −0.121 | 150 |
| rottlerin | | 3 | 0.209 | 53 | 0.355 | 40 |
| colchicine | | 2 | 0.192 | 54 | 0.391 | 37 |
| 5252917 | | 2 | 0.192 | 55 | 0.582 | 12 |
| fulvestrant | | 7 | 0.19 | 56 | 0.069 | 75 |
| arachidonyltrifluoromethane | | 2 | 0.185 | 57 | 0.239 | 51 |
| sulindac | | 2 | 0.177 | 58 | 0.207 | 57 |
| depudecin | | 2 | 0.176 | 59 | 0.22 | 56 |
| haloperidol | | 6 | 0.172 | 60 | 0.131 | 66 |
| felodipine | | 3 | 0.171 | 61 | 0.171 | 60 |
| troglitazone | | 6 | 0.167 | 62 | 0.42 | 34 |
| nifedipine | | 2 | 0.149 | 63 | 0.153 | 65 |
| rofecoxib | | 6 | 0.148 | 64 | 0.088 | 72 |
| valproic acid | | 18 | 0.136 | 65 | 0.086 | 73 |
| ionomycin | | 3 | 0.129 | 66 | 0.556 | 17 |
| wortmannin | | 8 | 0.126 | 67 | −0.324 | 155 |
| thalidomide | | 2 | 0.121 | 68 | −0.256 | 153 |
| ciclosporin | | 2 | 0.121 | 69 | 0.414 | 35 |
| celecoxib | | 5 | 0.114 | 70 | −0.01 | 140 |
| iloprost | | 3 | 0.101 | 71 | 0.098 | 70 |
| cobalt chloride | | 3 | 0.1 | 72 | 0.269 | 48 |
| genistein | | 7 | 0.097 | 73 | 0.153 | 64 |
| TTNPB | | 2 | 0.095 | 74 | 0 | 97 |
| SC-58125 | | 4 | 0.09 | 75 | −0.112 | 149 |
| indometacin | | 4 | 0.087 | 76 | 0.228 | 53 |
| monastrol | | 8 | 0.069 | 77 | 0.226 | 55 |
| copper sulfate | | 4 | 0.066 | 78 | 0.113 | 68 |
| pirinixic acid | | 5 | 0.055 | 79 | 0.226 | 54 |
| alpha-estradiol | | 6 | 0.045 | 80 | −0.066 | 145 |
| tacrolimus | | 3 | 0 | 81 | 0 | 110 |
| topiramate | | 1 | 0 | 82 | 0 | 115 |
| oxamic acid | | 1 | 0 | 83 | −0.654 | 163 |
| 3-aminobenzamide | | 1 | 0 | 84 | −0.587 | 160 |
| 1,5-isoquinolinediol | | 1 | 0 | 85 | −0.608 | 162 |
| rosiglitazone | | 4 | 0 | 86 | 0.098 | 69 |
| demecolcine | | 1 | 0 | 87 | 0 | 121 |
| 12,13-EODE | | 1 | 0 | 88 | 0 | 124 |
| yohimbine | | 1 | 0 | 89 | 0 | 126 |
| colforsin | | 2 | 0 | 90 | 0 | 129 |
| dexverapamil | | 1 | 0 | 91 | 0 | 133 |
| quinpirole | | 1 | 0 | 92 | 0 | 134 |
| probucol | | 1 | 0 | 93 | 0.332 | 43 |
| phentolamine | | 1 | 0 | 94 | 0 | 135 |
| deferoxamine | | 3 | 0 | 95 | −0.175 | 151 |
| 3-hydroxy-DL-kynurenine | | 1 | 0 | 96 | 0 | 136 |
| exemestane | | 1 | 0 | 97 | 0.427 | 33 |
| 5279552 | | 2 | 0 | 98 | 0.556 | 18 |
| paclitaxel | | 1 | 0 | 99 | 0.319 | 45 |
| 5109870 | | 1 | 0 | 100 | 0 | 88 |
| dopamine | | 1 | 0 | 101 | −0.596 | 161 |
| 4,5-dianilinophthalimide | | 2 | 0 | 102 | 0 | 89 |
| LY-294002 | | 17 | 0 | 103 | −0.512 | 158 |
| 5151277 | | 1 | 0 | 104 | 0 | 112 |
| fisetin | | 1 | 0 | 105 | 0 | 91 |
| decitabine | | 1 | 0 | 106 | 0 | 92 |
| blebbistatin | | 2 | 0 | 107 | 0 | 87 |
| fludrocortisone | | 2 | 0 | 108 | 0 | 98 |
| clofibrate | | 2 | 0 | 109 | 0 | 93 |
| DL-PPMP | | 1 | 0 | 110 | 0 | 138 |
| prednisolone | | 1 | 0 | 111 | 0 | 95 |
| flufenamic acid | | 1 | 0 | 112 | 0 | 96 |
| sirolimus | | 10 | 0 | 113 | −0.283 | 154 |
| amitriptyline | | 1 | 0 | 114 | 0 | 139 |
| fasudil | | 2 | 0 | 115 | 0 | 100 |
| tomelukast | | 1 | 0 | 116 | 0.388 | 38 |
| nitrendipine | | 1 | 0 | 117 | 0 | 99 |
| phenformin | | 1 | 0 | 118 | 0.355 | 39 |
| dimelhyloxalylglycine | | 1 | 0 | 119 | 0.326 | 44 |
| minocycline | | 1 | 0 | 120 | 0 | 104 |
| phenyl biguanide | | 1 | 0 | 121 | 0 | 105 |
| U0125 | | 1 | 0 | 122 | 0 | 106 |
| dexamethasone | | 3 | 0 | 123 | 0 | 108 |
| ikarugamycin | | 3 | 0 | 124 | 0.172 | 59 |
| mesalazine | | 1 | 0 | 125 | 0 | 109 |
| imatinib | | 2 | 0 | 126 | 0.163 | 62 |
| splitomicin | | 1 | 0 | 127 | −0.525 | 159 |
| chlorpropamide | | 2 | 0 | 128 | 0 | 111 |
| sulfasalazine | | 1 | 0 | 129 | 0 | 86 |
| doxycycline | | 1 | 0 | 130 | 0 | 84 |
| 5114445 | | 1 | 0 | 131 | 0 | 82 |
| 5152487 | | 1 | 0 | 132 | 0 | 81 |
| 5149715 | | 1 | 0 | 133 | 0 | 80 |
| 5186223 | | 1 | 0 | 134 | 0 | 79 |
| 5186324 | | 1 | 0 | 135 | 0 | 119 |
| sulindac sulfide | | 1 | 0 | 136 | 0 | 77 |
| gefitinib | | 1 | 0 | 137 | −0.713 | 164 |
| staurosporine | | 4 | 0 | 138 | 0.092 | 71 |
| acetylsalicylic acid | | 3 | 0 | 139 | 0.154 | 63 |
| tolbutamide | | 1 | 0 | 140 | 0.497 | 24 |
| tyrphostin AG-825 | | 1 | 0 | 141 | 0 | 137 |
| verapamil | | 1 | 0 | 142 | 0 | 131 |
| monensin | | 1 | 0 | 143 | 0.454 | 27 |
| sodium phenylbutyrate | | 7 | −0.029 | 144 | −0.083 | 146 |
| tetraethylenepentamine | | 6 | −0.092 | 145 | −0.039 | 144 |
| metformin | | 5 | −0.108 | 146 | −0.029 | 142 |
| bucladesine | | 3 | −0.144 | 147 | 0 | 123 |
| diclofenac | | 2 | −0.165 | 148 | −0.197 | 152 |
| estradiol | | 10 | −0.166 | 149 | 0.017 | 76 |
| tretinoin | | 8 | −0.206 | 150 | −0.084 | 147 |
| arachidonic acid | | 3 | −0.222 | 151 | 0 | 120 |
| NU-1025 | | 2 | −0.222 | 152 | −0.362 | 156 |
| exisulind | | 2 | −0.277 | 153 | −0.038 | 143 |
| clozapine | | 2 | −0.291 | 154 | 0 | 118 |
| MK-886 | | 2 | −0.336 | 155 | 0 | 94 |
| Y-27632 | | 2 | −0.353 | 156 | 0 | 107 |
| prazosin | | 2 | −0.358 | 157 | 0.231 | 52 |
| 5230742 | | 2 | −0.401 | 158 | 0.203 | 58 |
| quercetin | | 2 | −0.431 | 159 | −0.111 | 148 |
| benserazide | | 2 | −0.441 | 160 | 0 | 78 |
| N-phenylanthranilic acid | | 1 | −0.472 | 161 | 0 | 128 |
| butirosin | | 1 | −0.55 | 162 | 0 | 132 |
| tioguanine | | 1 | −0.613 | 163 | 0 | 102 |
| phenanthridinone | | 1 | −1 | 164 | 0.429 | 32 |

This file contains the full list of compounds in our expression profile database that showed enrichment or under-enrichment with the celastrol and gedunin signatures (1.25 μM celastrol at 6 h, 20 μM gedunin at 6 hours).

### Celastrol and Gedunin Inhibit Hsp90 Pathway

Having used the Connectivity Map to generate the hypothesis that celastrol and gedunin function as HSP90 inhibitors, we next sought to validate this hypothesis. Since AR is an HSP90 client protein, celastrol- and gedunin-mediated inhibition of HSP90 could explain the observed suppression of androgen signaling. HSP90 inhibition induces degradation of AR and other client proteins and thereby targets multiple, cooperative oncogenic signaling pathways.

We first asked whether celastrol and gedunin decrease the levels of AR itself. Both celastrol and gedunin were found to decrease AR protein levels in a concentration-dependent manner (FIG. 7C). Celastrol decreased AR levels in LNCaP cells at 0.5 μM and above, while gedunin decreases their levels at 10 μM and above. Almost complete ablation of AR levels was seen at 1 μM celastrol and 20 μM gedunin. These concentration-dependent effects on AR are consistent with the observed inhibitory effects on AR-mediated signaling. Notably, HSP90 inhibitors 17-AAG and geldanamycin suppressed the androgen signaling signature as well.

To more broadly establish the effects of celastrol and gedunin on the HSP90 pathway, we tested whether these compounds decrease the protein levels of other HSP90 clients. Celastrol and gedunin treatment lowered the protein levels of FLT3, EGFR, and BCR-ABL1 in a concentration-dependent manner in several cell lines (FIG. 7D). These findings demonstrate that celastrol and gedunin decrease the levels of a range of HSP90 client proteins.

Given their inhibition of HSP90 clients, we next asked whether celastrol and gedunin affect HSP90 activity itself. To assess the effects on HSP90 activity within a cellular context, we treated LNCaP and K562 cells with celastrol or gedunin for 24 hr and subsequently tested the cellular HSP90's ATP-binding activity. ATP-binding activity was assayed by ATP-polyacrylamide pulldown of HSP90 from cell lysates, followed by western blot-based quantification (Bali et al., 2005). This assay identifies HSP90 inhibition, both direct and indirect, that alters HSP90 ATP-binding activity in cell lines (Bali et al., 2005, Soti et al., 2002). We found that celastrol and gedunin treatment inhibited the ATP-binding activity of HSP90α in both cell lines (FIG. 7E). In contrast, compound treatment did not affect the ATP-binding activity of the kinases CSK and DDR1, which are not HSP90 clients. The decrease in ATP binding by HSP90 cannot be accounted for by changes in HSP90 level (FIG. 7E). Celastrol and gedunin therefore inhibit HSP90 activity itself in a cellular context, either directly or indirectly.

Celastrol, as the more potent compound, was then tested for effects on HSP90's functional interactions with cochaperones. Consistent with its reduction of HSP90 ATP-binding activity, celastrol treatment reduced HSP90 interaction with the cochaperone p23 in SKBR-3 cells, as determined by coimmunoprecipitation with HSP90 (FIG. 7F). The N-terminal inhibitor PU-H71 (He et al., 2006, Vilenchik et al., 2004) had the same effect. p23 interacts with the ATP-bound form of HSP90 and helps stabilize the mature steroid receptor-HSP90 complex (Felts et al., 2003). Geldanamycin and other HSP90 inhibitors are known to inhibit p23 association with HSP90 (Felts et al., 2003). In contrast, HSP90-HOP interaction was unaltered by celastrol and other HSP90 inhibitors (FIG. 7F). Thus, celastrol inhibits the functional interactions of HSP90 and suppresses HSP90 client levels.

Celastrol and Gedunin Modulate HSP90 Activity by a Mechanism that is Distinct from that of Existing HSP90 ATP-Binding Pocket Inhibitors

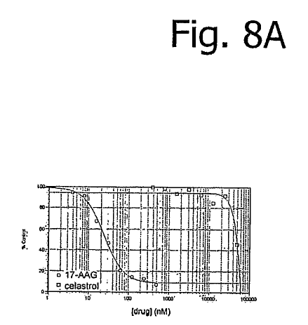

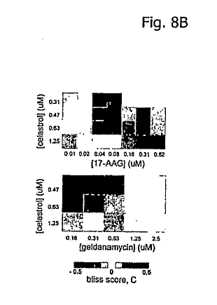

Since celastrol and gedunin inhibit HSP90 pathway function, we asked whether celastrol and gedunin act by competitively binding to the ATP-binding pocket of HSP90, the mechanism common to most HSP90 inhibitors (Whitesell et al., 2005). We first tested whether celastrol or gedunin could compete with Cy3B-geldanamycin for binding to the ATP-binding pocket of purified HSP90α by fluorescence polarization assay (Kim et al., 2004, Llauger-Bufi et al., 2003). In contrast to the earlier ATP-binding activity assay, this experiment tested the ability of celastrol and gedunin to directly inhibit small molecule binding to the ATP pocket of purified HSP90 when combined in vitro. Neither celastrol nor gedunin significantly competed with geldanamycin binding to recombinant HSP90α at concentrations up to ˜100 μM, with compound addition before and after geldanamycin addition (FIG. 8A). The N-terminal inhibitors 17-AAG and PU-H71, on the other hand, competed with geldanamycin binding at low concentrations in vitro (FIG. 8A) (He et al., 2006, Vilenchik et al., 2004). These results indicate that celastrol and gedunin act on HSP90 by a different mechanism than existing N-terminal HSP90 inhibitors.

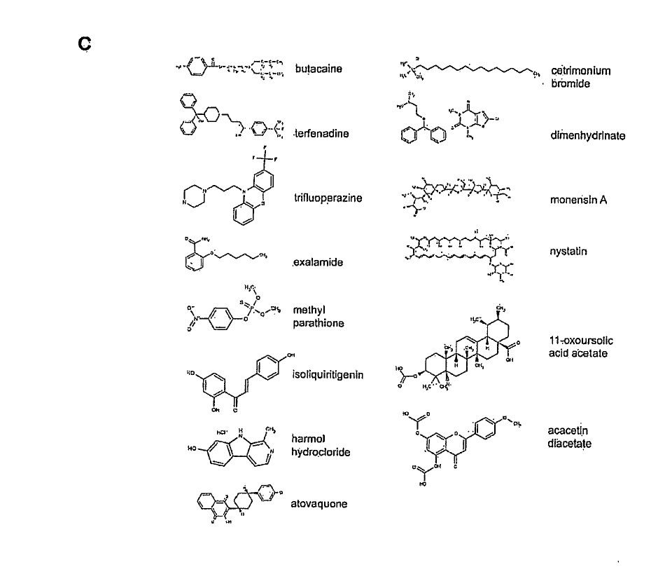

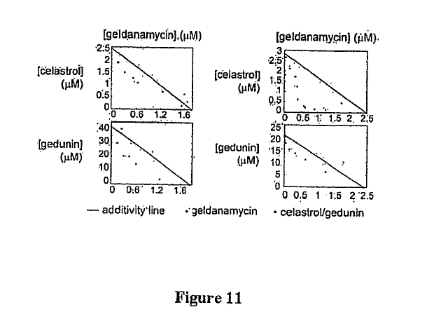

If celastrol and gedunin act on HSP90 function via a distinct mechanism from HSP90 ATP-binding site inhibition (Bagatell et al., 2005), they might act synergistically with existing HSP90 inhibitors. We therefore tested the combinatorial effects of these compounds with HSP90 inhibitors on HSP90 client signaling and viability. We found that celastrol and gedunin show mild synergy with geldanamycin and 17-AAG in inhibiting the androgen signaling signature, as shown by isobologram analysis (FIGS. 8B and 11). Celastrol and gedunin also synergistically inhibit cell growth, assayed by ATP level, with geldanamycin and 17-AAG at low concentrations (FIGS. 8C and 11). Celastrol and gedunin therefore act synergistically with existing modes of HSP90 ATP-binding site inhibition to inhibit HSP90 client signaling and viability in a cellular context, consistent with their inhibition of HSP90 via a distinct mechanism.

### Discussion

Chemical genomics has the potential to identify modulators of complex cancer phenotypes and predict their activities with little prior knowledge about the underlying mechanisms. Here we report a chemical genomic screen for modulators of AR-mediated signaling modulators, a critical cancer signaling pathway. To investigate the activity of the resulting celastrol and gedunin family of hits, a gene expression-based approach was used to identify similar known drug activities and predicted that these compounds act as HSP90 pathway inhibitors. We then validated this hypothesis by demonstrating that celastrol and gedunin destabilize HSP90 clients including AR and inhibit HSP90 function. Moreover, celastrol and gedunin act outside the HSP90 ATP-binding pocket targeted by most HSP90 inhibitors and act synergistically with these inhibitors.

Given the central role that HSP90 and its clients play in cancer biology, celastrol and gedunin compounds represent a significant new set of HSP90 pathway modulators. The work presented here identifies celastrol- and gedunin-mediated inhibition of HSP90 client activity including AR (Yang et al., 2006) and illustrates its broad downstream effects on AR-regulated gene expression (Georget et al., 2002, Waza et al., 2005). Celastrol and gedunin are further shown to affect HSP90 activity and interactions. The decrease in HSP90's ATP-binding activity and HSP90-p23 interaction could result from a shift to the ADP complexed form of HSP90, which directs client proteins to the proteasome (Bali et al., 2005, Felts et al., 2003, Soti et al., 2002). Indeed, celastrol treatment is known to cause accumulation of ubiquitinated proteins (Yang et al., 2006); such accumulation can result from HSP90 inhibition and stress response, and the subsequent redirection of proteins through the proteasomal pathway (Mimnaugh et al., 2004). Consistent with HSP90-inhibitory activity, celastrol has also been shown to induce HSP70 levels (Westerheide et al., 2004), a hallmark of HSP90 inhibition by existing ansamycin antibiotic HSP90 inhibitors as well as stress and heat shock response (Murakami et al., 1991). Celastrol has additionally been shown to suppress hERG potassium channel activity by inhibiting hERG maturation (Sun et al., 2006), which is seen with existing HSP90 inhibitors and is hypothesized to result from HSP90 inhibition (Ficker et al., 2003). Both celastrol and existing HSP90 inhibitors appear to be active in neurodegenerative disease models (Wang et al., 2005, Waza et al., 2005) where, notably, 17-AAG inhibits neurodegeneration induced by polyglutamine expansion of AR. Last, both celastrol and gedunin also have noted antimalarial activity, as have other HSP90 inhibitors (Figueiredo et al., 1998, MacKinnon et al., 1997). These observations can be unified by the present discovery of celastrol and gedunin's HSP90-inhibitory activity.

Celastrol and gedunin compounds have the potential to provide new modes of HSP90 inhibition. Celastrol and gedunin act outside the N-terminal ATP-binding pocket of HSP90 and therefore inhibit HSP90 function by a mechanism that is distinct from that of most existing HSP90 inhibitors. Few compounds inhibit HSP90 through mechanisms outside this N-terminal domain (Bali et al., 2005, Kovacs et al., 2005, Marcu et al., 2000). Only two other existing drugs, cisplatin and novobiocin, act directly on HSP90 outside this fold by binding the C-terminal domain, and even these only induce HSP90 inhibition at high concentrations at which other mechanisms of action likely predominate (Marcu et al., 2000, Whitesell et al., 2005). While our work demonstrates that celastrol and gedunin inhibit HSP90 function by acting outside the ATP-binding pocket, it remains to be determined whether they act directly or indirectly on HSP90. Induction of heat shock response or other regulatory mechanisms could, for example, indirectly inhibit HSP90 function. Future work may address the mechanistic details of this HSP90 modulation.

Because celastrol and gedunin inhibit HSP90 function through a different mechanism than N-terminal HSP90 inhibitors, celastrol and gedunin compounds may have significant therapeutic and scientific potential. Triterpenoid derivatives of the celastrol and gedunin family compounds may serve as a starting point for development of drugs that prove useful both in combination with existing HSP90 inhibitors or alone, in the advent of resistance against existing inhibitors. Scientifically, celastrol and gedunin may afford further insight into HSP90 biology by providing tools to probe HSP90 function; several significant HSP90 interactors have been discovered through synthetic screens for genes that confer hypersensitivity to geldanamycin-mediated inhibition, for example (Zhao et al., 2005). Thus, celastrol and gedunin offer a unique window into HSP90 inhibition with broad therapeutic and scientific possibilities.

Beyond HSP90 modulation, this work addresses a significant problem in chemical biology: the discovery of modulators of complex cancer phenotypes and the molecular activities underlying these modulators. We have demonstrated a combined chemical genomic approach to compound discovery and characterization based wholly on gene expression. This strategy provides a useful endpoint for drug and activity screening, since assaying associative effects can serve as a proxy for assaying causal effects. Nonetheless, the strength of the gene expression, as opposed to other readouts, as an assay may vary depending on the biology underlying the state being studied.

Significantly, we have applied a robust approach for chemical activity prediction that uses gene expression signature enrichment analysis to identify similar known drug activities. Compendia of gene expression profiles have been previously used to identify gene targets of drug perturbations in both bacteria (Gardner et al., 2003) and yeast (di Bernardo et al., 2005, Hughes et al., 2000, Parsons et al., 2004), but such work has not previously been extended to mammalian systems. The approach presented here identifies a target pathway of two uncharacterized compounds in a manner robust to ad hoc experimental decisions including cell context and treatment parameters. Notably, though some connectivity is dependent upon appropriate context (for example, estrogen signaling requiring estrogen receptor expression), there appears to be cell line independence in the majority of the cases examined (Lamb et al., 2006). One caveat to this approach is that it requires that the activity of query compounds be represented among the profiled drug activities. Our approach additionally may not distinguish between direct and indirect compound activities in all cases, though this may afford a nuanced view. In sum, this work demonstrates a promising chemical genomic strategy for discovering modulators of complex cancer phenotypes and subsequently establishing their mechanisms of action.

### Experimental Procedures

### Reagents and Cell Lines

Celastrol (Calbiochem) and gedunin (Gaia Chemicals) were solubilized in DMSO. LNCaP.FGC cells (ATCC) were grown in RPMI 1640 (ATCC) with 10% FBS. Ba/F3 cells stably expressing human FLT3, EGFR, and BCR-ABL1 were grown in RPMI 1640, 10% FBS, and 2 ng/ml IL-3. SKBr3 cells were grown in a 1:1 DME:F12 plus 10% FBS.

### Gene Expression Profiling and Analysis

The mRNA expression profiles of celastrol- and gedunin-treated cells were determined by Affymetrix U133A microarray analysis in triplicate. RNA was isolated by Trizol extraction from LNCaP cells treated with vehicle, 1.25 μM celastrol, or 20 μM gedunin (1) for 24 hr in RPMI, 10% charcoal-stripped FBS, and 1 nM R1881 or vehicle, following androgen deprivation in charcoal-stripped media for 2 days, and (2) for 6 hr in RPMI with 10% FBS. IVT, labeling, hybridization, and washing were carried out on the Affymetrix High-Throughput Array platform using HT_HG-U133A preproduction arrays (early access version; part number 520276) for all but the 24 hr gedunin samples. U133A version 2 arrays were used for the 24 hr gedunin samples for technical reasons. Raw data were processed by RMA. For hierarchical clustering, a 169 probe set of androgen-regulated genes was defined and used to perform average linkage clustering (see below). Raw data are available at www.broad.mit.edulcgi-bin/cancer/publications/pub_menu.cgi/ and NCBI's Gene Expression Omnibus (GEO; www.ncbi.nlm.nih.gov/geo/; accession numbers GSE5505 to GSE5508).

### Gene Expression Signature Analysis

### Androgen Signaling Signature

The androgen signaling signature was developed from independent Affymetrix U133A profiles of LNCaP cells treated with 0.1 nM R1881 over a 24 hr time course (Febbo et al., 2005). Class neighbors analysis was used to identify genes that are differentially expressed upon R1881 androgen treatment relative to vehicle by the SNR metric (Golub et al., 1999, Reich et al., 2006). The top marker genes were tested for differential expression between androgen-stimulated and -deprived states by GE-HTS assay. The 27 genes with the most robust discrimination by SNR were chosen for the GE-HTS androgen signaling signature (Table 3). Two normalization controls, SRP72 and KIAA0676, were selected from genes with moderate expression levels that varied little over the R1881 time course.

TABLE 3
GE-HTS probe sequences.

| | probe based | |
|---|---|---|
| Symbol | on | UG and description |
| FKBP5 | NM_004117 | Hs.407190, FK506-binding protein 5 |
| Kallikrein2 | AF188745. | Hs.181350 kallikrein 2, prostatic |
| ELL2 | NM_012081.3 | Hs.192221, RNA POLYMERASE II, ELONGATION FACTOR, 2 |
| TMPRSS2 | NM_005656.2 | Hs.439309, Transmembrane protease, serine 2 (TMPRSS2) |
| ZBTB10 | NM_023929.2 | Hs.205742, Zinc finger and BTB domain containing 10 (ZBTB10) |
| BM039 | NM_018455.3 | Hs.283532 uncharacterized bone marrow protein BM039 |
| TMEPAI | NM_020182.3 | Hs.517155, Transmembrane, prostate androgen induced (TMEPAI) |
| NKX3-1 | AF247704 | Hs.55999 NK3 transcription factor related, locus 1 (NKX3-1) |
| KLK3 | NM_001648. | Hs.171995 kallikrein 3, (prostate specific antigen) |
| EAF2 | NM_018456.4 | Hs.477325 ELL associated factor 2 (EAF2) |
| MED28 | AK024944.1 | Hs.33032 Mediator of RNA polymerase II txn subunit 28 homolog (MED28) |
| ABCC4 | NM_005845. | Hs.508423 ATP-binding cassette, sub-family C, member 4 (ABCC4) |
| SARG | NM_023938 | Hs.32417 Specifically androgen-regulated protein (SARG) |
| GNMT | NM_018960. | Hs.144914 glycine N-methyltransferase |
| MPHOSPH9 | X98258 | Hs.507175 M-phase phosphoprotein 9 (MPHOSPH9) |
| NNMT | NM_006169.1 | Hs.503911 nicotinamide N-methyltransferase |
| ADAM7 | AF215824 | Hs.166003 a disintegrin and metalloproteinase domain 7 |
| TNK1 | NM_003985.1 | Hs.203420 tyrosine kinase, non-receptor, 1 |
| MAN1A1 | BG287153. | Hs.102788 Mannosidase, alpha, class 1A, member 1 (MAN1A1) |
| GLRA2 | NM_002063. | Hs.2700 glycine receptor, alpha 2 |
| CD200 | AF063591 | Hs.79015 CD200 antigen (identified by monoclonal antibody MRC OX-2) |
| MAPRE2 | BE671156. | Hs.532824 Microtubule-associated protein, RP/EB family, 2 (MAPRE2) |
| PIP5K2B | NM_003559. | Hs.260603 Phosphatidylinositol-4-phosphate 5-kinase, type II, beta (PIP5K2B) |
| AR | NM_000044.2 | Hs.496240 Androgen receptor (DHT receptor) (AR) |
| SRP72 | NM_006947.2 | Hs.237825 signal recognition particle 72 kD |
| KIAA0676 | AK026096. | Hs.155829 KIAA0676 protein (KIAA0676) |
| ACSL3 | NM_004457.3 | Hs.471461 acyl-CoA synthetase long-chain family member 3 (ACSL3), variant 1 |
| MAF | AF055376.1 | Hs.134859 V-maf musculoaponeurotic fibrosarcoma oncogene homolog (MAF) |
| HERC3 | NM_014606.1 | Hs.35804 hect domain and RLD 3 |
| PTGER4 | NM_000958.2 | Hs.199248 prostaglandin E receptor 4 (subtype EP4) |

| Symbol | UG code | complement of 40 bp target |
|---|---|---|
| FKBP5 | Hs.407190 | TATGACGCCACGCCAAGGAGGGAAGAGTCCCAGTGAACTC |
| Kallikrein2 | Hs.181350 | CCTTGTGGAATGCAGCTGACCCAGCTGATAGAGGAAGTAG |
| ELL2 | Hs.192221 | AAGCTGCTACTCCTAGTAGGCCAAACGCTCAGGTTAAACA |
| TMPRSS2 | Hs.439309 | AGGGACCTTGGAAACAGTTGGCACTGTAAGGTGCTTGCTC |
| ZBTB10 | Hs.205742 | AGCTGGCACTAGTCAAGATGGAGGTGATGCTGGTACTTCA |
| BM039 | Hs.283532 | GGCGCAATACACCGCTTCTGGGTCAGGAGTTAGAAGCTAC |
| TMEPAI | Hs.517155 | CATCCCAAGACAGCCAGCAGGTTGTCTCTGGAAACGACCA |
| NKX3-1 | Hs.55999 | CACGTGCTGCTGACACCGACCGGAGTACTAGCCAGCACAA |
| KLK3 | Hs.171995 | GCTGGACACTGTCCATGAAGCACTGAGCAGAAGCTGGAGG |
| EAF2 | Hs.477325 | TAGTGAGGATAGTTCTAGTGACTCAGAAGATGAAGATTGC |
| MED28 | Hs.33032 | TATAGGGTGTTTGTAGAAGGGATAAATGGGTTACCTAATG |
| ABCC4 | Hs.508423 | ACTGCCAAAATACATTGACCGTAGTAGCTGTTCAACTCCT |
| SARG | Hs.32417 | TTGAAGAGATGCAAGAGGGCCCAGTGAGGACATCCGCCTC |
| GNMT | Hs.144914 | ATAAGAGTGACTTGACCAAGGACGTCACAACATCAGTGCT |
| MPHOSPH9 | Hs.507175 | TCCTGTGTCTACATTACAACGTACCAATCCAAGAAAGCAA |
| NNMT | Hs.503911 | AATCCTTTAAGGAGATCGTCGTCACTGACTACTCAGACCA |
| ADAM7 | Hs.166003 | GCAGATCCCAATCAAAGTGCCAAGTGAGCTTGAAGTTGGA |
| TNK1 | Hs.203420 | CATTTGATGCTGGTAGTATGGATTATGAGATGGACTAGCC |
| MAN1A1 | Hs.102788 | TACACATGTAAGTTGTATGGCAGTTTACAGAACTCAATGA |
| GLRA2 | Hs.2700 | GTGTGTCCTGAACAGTGTAGCTCAGGTCAGCTTGAACTTT |
| CD200 | Hs.79015 | CTAGAATCCTTGGTTCACTGCTGTCTTCATGTGTTCTATG |
| MAPRE2 | Hs.532824 | AGAGGGTATGGTTAGGTACGGGTCTTCCTGCCTCATTCCT |
| PIP5K2B | Hs.260603 | TTCGGAGCCTGCCACCCAGGCCCTCAGAACTGAGCCACAG |
| AR | Hs.496240 | CCCTTTCAGATGTCTTCTGCCTGTTATAACTCTGCACTAC |
| SRP72 | Hs.237825 | TCACATTGAACAGCTGTGAGACAGACATATTGAGATGCCT |
| KIAA0676 | Hs.155829 | TGCCAGGTCAGATGGAGACGCAGAACCTGCTGGTGCAAGC |
| ACSL3 | Hs.471461 | AGGTCTGTGTAAAGTAAGGGGAGTGTTAGGAGCAGCCAGG |
| MAF | Hs.134859 | TGAGTTACTAACTAACCACGCGTGTTGTTCCTATGTGCTT |
| HERC3 | Hs.35804 | AGGTTAATTGATAGACCCACCACCTCTTGCACTCTCGCTT |
| PTGER4 | Hs.199248 | GTCAGAAGTGCAGAATTGGGGCACTTAATGGTCACCTTGT |

| Symbol | upstream probe |
|---|---|
| FKBP5 | TAA TAC GAC TCA CTA TAG GGTACAAATCATCAATCACTTTAATCTATGACGCCACGCCAAGGAG |
| Kallikrein2 | TAA TAC GAC TCA CTA TAG GGTACACTTTCTTTCTTTCTTTCTTT CCTTGTGGAATGCAGCTGAC |
| ELL2 | TAA TAC GAC TCA CTA TAG GGCAATAAACTATACTTCTTCACTAA AAGCTGCTACTCCTAGTAGG |
| TMPRSS2 | TAA TAC GAC TCA CTA TAG GGCTACTATACATCTTACTATACTTT AGGGACCTTGGAAACAGTTG |
| ZBTB10 | TAA TAC GAC TCA CTA TAG GGATACTTCATTCATTCATCAATTCA AGCTGGCACTAGTCAAGATG |
| BM039 | TAA TAC GAC TCA CTA TAG GGAATCAATCTTCATTCAAATCATCA GTGTACTACTCCCAGACTCC |
| TMEPAI | TAA TAC GAC TCA CTA TAG GGAATCCTTTCTTTAATCTCAAATCA CATCCCAAGACAGCCAGCAG |
| NKX3-1 | TAA TAC GAC TCA CTA TAG GGTTCAATCATTCAAATCTCAACTTT CACGTGCTGCTGACACCGAC |
| KLK3 | TAA TAC GAC TCA CTA TAG GGTCAATTACCTTTTCAATACAATAC GCTGGACACTGTCCATGAAG |
| EAF2 | TAATACGACTCACTATAGGGCTTTTCAATTACTTCAAATCTTCATAGTGAGGATAGTTCTAGTG |
| MED28 | TAATACGACTCACTATAGGGTTACTCAAAATCTACACTTTTTCATATAGGGTGTTTGTAGAAGG |
| ABCC4 | TAA TAC GAC TCA CTA TAG GGCTTTTCAAATCAATACTCAACTTT ACTGCCAAAATACATTGACC |
| SARG | TAA TAC GAC TCA CTA TAG GGAATCTTACTACAAATCCTTTCTTT TTGAAGAGATGCAAGAGGGC |
| GNMT | TAA TAC GAC TCA CTA TAG GGCAATTTCATCATTCATTCATTTCA ATAAGAGTGACTTGACCAAG |
| MPHOSPH9 | TAA TAC GAC TCA CTA TAG GGCTTTTCATCTTTTCATCTTTCAAT TCCTGTGTCTACATTACAAC |
| NNMT | TAA TAC GAC TCA CTA TAG GGTCAATCATTACACTTTTCAACAAT AATCCTTTAAGGAGATCGTC |
| ADAM7 | TAA TAC GAC TCA CTA TAG GGTACACAATCTTTTCATTACATCAT GCAGATCCCAATCAAAGTGC |
| TNK1 | TAATACGACTCACTATAGGGCTTTCTACATTATTCACAACATTACATTTGATGCTGGTAGTATG |
| MAN1A1 | TAA TAC GAC TCA CTA TAG GGCTATCTTCATATTTCACTATAAAC TACACATGTAAGTTGTATGG |
| GLRA2 | TAATACGACTCACTATAGGGTCATTTCACAATTCAATTACTCAAGTGTGTCCTGAACAGTGTAG |
| CD200 | TAA TAC GAC TCA CTA TAG GGCTTCTCATTAACTTACTTCATAAT CTAGAATCCTTGGTTCACTG |
| MAPRE2 | TAA TAC GAC TCA CTA TAG GGAAACAAACTTCACATCTCAATAAT AGAGGGTATGGTTAGGTACG |
| PIP5K2B | TAA TAC GAC TCA CTA TAG GGTCATCAATCTTTCAATTTACTTAC TTCGGAGCCTGCCACCCAGG |
| AR | TAA TAC GAC TCA CTA TAG GGCAATATACCAATATCATCATTTAC CCCTTTCAGATGTCTTCTGC |
| SRP72 | TAA TAC GAC TCA CTA TAG GGTCATTTCAATCAATCATCAACAATTCACATTGAACAGCTGTGAG |
| KIAA0676 | TAA TAC GAC TCA CTA TAG GGTAATTATACATCTCATCTTCTACATGCCAGGTCAGATGGAGACG |
| ACSL3 | TAATACGACTCACTATAGGG CTATTACACTTTAAACATCAATAC AGGTCTGTGTAAAGTAAGGG |
| MAF | TAATACGACTCACTATAGGG CTATCTATCTAACTATCTATATCA TGAGTTACTAACTAACCACG |
| HERC3 | TAATACGACTCACTATAGGG AATCTACACTAACAATTTCATAAC AGGTTAATTGATAGACCCAC |
| PTGER4 | TAATACGACTCACTATAGGG CTATCTTTAAACTACAAATCTAAC GTCAGAAGTGCAGAATTGGG |

| Symbol | downstream probe | barcode/Flexmap tag number |
|---|---|---|
| FKBP5 | GGAAGAGTCCCAGTGAACTCTCC CTT TAG TGA GGG TTA AT | LUA-11 GATTAAAGTGATTGATGATTTGTA |
| Kallikrein2 | CCAGCTGATAGAGGAAGTAGTCC CTT TAG TGA GGG TTA AT | LUA-12 AAAGAAAGAAAGAAAGAAAGTGTA |
| ELL2 | CCAAACGCTCAGGTTAAACATCC CTT TAG TGA GGG TTA AT | LUA-13 TTAGTGAAGAAGTATAGTTTATTG |
| TMPRSS2 | GCAGTGTAAGGTGCTTGCTCTCC CTT TAG TGA GGG TTA AT | LUA-14 AAAGTATAGTAAGATGTATAGTAG |
| ZBTB10 | GAGGTGATGCTGGTACTTCATCC CTT TAG TGA GGG TTA AT | LUA-15 TGAATTGATGAATGAATGAAGTAT |
| BM039 | GTACGCCTTCACGTCCTCCTTCC CTT TAG TGA GGG TTA AT | LUA-16 TGATGATTTGAATGAAGATTGATT |
| TMEPAI | GTTGTCTCTGGAAACGACCATCC CTT TAG TGA GGG TTA AT | LUA-21 TGATTTGAGATTAAAGAAAGGATT |
| NKX3-1 | CGGAGTACTAGCCAGCACAATCC CTT TAG TGA GGG TTA AT | LUA-23 AAAGTTGAGATTTGAATGATTGAA |
| KLK3 | CACTGAGCAGAAGCTGGAGGTCC CTT TAG TGA GGG TTA AT | LUA-24 GTATTGTATTGAAAAGGTAATTGA |
| EAF2 | ACTCAGAAGATGAAGATTGCTCCCTTTAGTGAGGGTTAAT | LUA-25 TGAAGATTTGAAGTAATTGAAAAG |
| MED28 | GATAAATGGGTTACCTAATGTCCCTTTAGTGAGGGTTAAT | LUA-26 TGAAAAAGTGTAGATTTTGAGTAA |
| ABCC4 | GTAGTAGCTGTTCAACTCCTTCC CTT TAG TGA GGG TTA AT | LUA-27 AAAGTTGAGTATTGATTTGAAAAG |
| SARG | CCAGTGAGGACATCCGCCTCTCC CTT TAG TGA GGG TTA AT | LUA-29 AAAGAAAGGATTTGTAGTAAGATT |
| GNMT | GACGTCACAACATCAGTGCTTCC CTT TAG TGA GGG TTA AT | LUA-35 TGAAATGAATGAATGATGAAATTG |
| MPHOSPH9 | GTACCAATCCAAGAAAGCAATCC CTT TAG TGA GGG TTA AT | LUA-37 ATTGAAAGATGAAAAGATGAAAAG |
| NNMT | GTCACTGACTACTCAGACCATCC CTT TAG TGA GGG TTA AT | LUA-38 ATTGTTGAAAAGTGTAATGATTGA |
| ADAM7 | CAAGTGAGCTTGAAGTTGGATCC CTT TAG TGA GGG TTA AT | LUA-39 ATGATGTAATGAAAAGATTGTGTA |
| TNK1 | GATTATGAGATGGACTAGCCTCCCTTTAGTGAGGGTTAAT | LUA-40 TAATGTTGTGAATAATGTAGAAAG |
| MAN1A1 | CAGTTTACAGAACTCAATGATCC CTT TAG TGA GGG TTA AT | LUA-42 GTTTATAGTGAAATATGAAGATAG |
| GLRA2 | CTCAGGTCAGCTTGAACTTTTCCCTTTAGTGAGGGTTAAT | LUA-45 TTGAGTAATTGAATTGTGAAATGA |
| CD200 | CTGTCTTCATGTGTTCTATGTCC CTT TAG TGA GGG TTA AT | LUA-47 ATTATGAAGTAAGTTAATGAGAAG |
| MAPRE2 | GGTCTTCCTGCCTCATTCCTTCC CTT TAG TGA GGG TTA AT | LUA-48 ATTATTGAGATGTGAAGTTTGTTT |
| PIP5K2B | CCCTCAGAACTGAGCCAGAGTCC CTT TAG TGA GGG TTA AT | LUA-49 GTAAGTAAATTGAAAGATTGATGA |
| AR | CTGTTATAACTCTGCACTACTCC CTT TAG TGA GGG TTA AT | LUA-50 GTAAATGATGATATTGGTATATTG |
| SRP72 | ACAGACATATTGAGATGCCTTCC CTT TAG TGA GGG TTA AT | LUA-51 ATTGTTGATGATTGATTGAAATGA |
| KIAA0676 | CAGAACCTGCTGGTGCAAGCTCC CTT TAG TGA GGG TTA AT | LUA-53 TGTAGAAGATGAGATGTATAATTA |
| ACSL3 | GAGTGTTAGGAGCAGCCAGG TCCCTTTAGTGAGGGTTAAT | LUA-92 GTATTGATGTTTAAAGTGTAATAG |
| MAF | CGTGTTGTTCCTATGTGCTT TCCCTTTAGTGAGGGTTAAT | LUA-78 TGATATAGATAGTTAGATAGATAG |
| HERC3 | CACCTCTTGCACTCTCGCTT TCCCTTTAGTGAGGGTTAAT | LUA-99 GTTATGAAATTGTTAGTGTAGATT |
| PTGER4 | GCACTTAATGGTCACCTTGT TCCCTTTAGTGAGGGTTAAT | LUA-100 GTTAGATTTGTAGTTTAAAGATAG |

### Celastrol and Gedunin Signatures

The celastrol and gedunin signatures were developed from RMA-processed microarray data from LNCaP cells treated with 1.25 μM celastrol or 20 μM gedunin for 6 hours. Comparative marker selection was used to identify markers that distinguished celastrol- and/or gedunin-treated samples from vehicle-treated samples by the median SNR (Golub et al., 1999). The top 50 markers that increased and decreased relative to vehicle-treated controls were used as the signatures.

### GE-HTS Androgen Signaling Signature Assay

The GE-HTS assay was carried out as described (Peck et al., 2006) using AR signature probes (Table 3).

### GE-HTS and Viability Screens

NINDS, Biomol, and SpecPlus libraries (www.broad.mit.edu/chembio/platform/screening/compound_libraries/index.htm/) were screened using GE-HTS androgen signaling and viability assays. After 2 days androgen deprivation, LNCaP cells were treated with compounds (˜20 μM) or vehicle (DMSO) plus 1 nM R1881 for 24 hr for the GE-HTS screen and for 3 days for the viability screen. Raw GE-HTS expression levels were filtered and normalized as described herein. Compounds were scored by weighted and unweighted “summed score” metrics, KNN classifier, and naive Bayes classifier to identify candidate modulators that induced the androgen deprivation signature. For heat map visualization, screen data were normalized between libraries using the mean SRP72 value of the androgen-deprived vehicle controls. Viability and soft agar assays

Adherent cell growth was measured by luminescent assay of ATP level (CellTiterGlo, Promega). LNCaP cells were grown in charcoal-stripped media for 2 days prior to simultaneous treatment with 1 nM R1881 and the relevant compound. Synergy was assessed by analyzing the IC50 of one drug over a range of concentrations of the other drug and vice versa. The resulting concentration pairs were visualized by isobologram (Gessner, 1995). Anchorage independence was measured by soft agar assay (Hahn et al., 1999). Compounds were added to both agar layers. Colonies were scored after 3 weeks.

### Connectivity Map Analysis for Drug Activity

The current version of the Connectivity Map data set (build01) contains genome-wide expression data for 453 treatment and vehicle control pairs, representing 164 distinct small molecules. Cell treatments and Affymetrix profiling were predominantly carried out in MCF7 cells with 6 hr treatments as detailed (Table 4) (Lamb et al., 2006). Enrichment of the induced and repressed genes of a signature within each Connectivity Map treatment profile was estimated with a metric based on the Kolmogorov-Smirnov statistic as described (Lamb et al., 2003, Lamb et al., 2006). Connectivity Map data are available at www.broad.mit.edu/cmap/ and GEO (accession number GSE5258).

TABLE 4
Connectivity map data and treatment descriptions.

| in- | | | concen- | dura- | | | | |
|---|---|---|---|---|---|---|---|---|
| stance | batch | | tration | tion | | | | |
| id | id | cmap_name | (M) | (h) | cell¹ | array² | perturbation_scan_id | vehicle_scan_id³ |
| 1 | 1 | metformin | 0.00001 | 6 | MCF7 | HG-U133A | EC2003090503AA | EC2003090502AA |
| 2 | 1 | metformin | 0.00001 | 6 | MCF7 | HG-U133A | EC2003090504AA | EC2003090502AA |
| 3 | 1 | metformin | 0.0000001 | 6 | MCF7 | HG-U133A | EC2003090505AA | EC2003090502AA |
| 4 | 1 | metformin | 0.001 | 6 | MCF7 | HG-U133A | EC2003090506AA | EC2003090502AA |
| 21 | 2 | phenformin | 0.00001 | 6 | MCF7 | HG-U133A | EC2003091104AA | EC2003091102AA |
| 22 | 2 | phenyl | 0.00001 | 6 | MCF7 | HG-U133A | EC2003091105AA | EC2003091102AA |
| | | biguanide | | | | | | |
| 23 | 2 | valproic acid | 0.001 | 6 | MCF7 | HG-U133A | EC2003091106AA | EC2003091102AA |
| 61 | 2a | metformin | 0.00001 | 6 | MCF7 | HG-U133A | EC2003091103AA | EC2003091102AA |
| 121 | 5 | estradiol | 0.00000001 | 6 | MCF7 | HG-U133A | EC2003092303AA | EC2003092302AA |
| 122 | 5 | alpha- | 0.00000001 | 6 | MCF7 | HG-U133A | EC2003092304AA | EC2003092302AA |
| | | estradiol | | | | | | |
| 123 | 5 | dexamethasone | 0.000001 | 6 | MCF7 | HG-U133A | EC2003092305AA | EC2003092302AA |
| 124 | 5 | mesalazine | 0.0001 | 6 | MCF7 | HG-U133A | EC2003092306AA | EC2003092302AA |
| 141 | 6 | chlorpropamide | 0.0001 | 6 | MCF7 | HG-U133A | EC2003092503AA | EC2003092502AA |
| 142 | 6 | tolbutamide | 0.0001 | 6 | MCF7 | HG-U133A | EC2003092504AA | EC2003092502AA |
| 143 | 6 | tamoxifen | 0.000001 | 6 | MCF7 | HG-U133A | EC2003092505AA | EC2003092502AA |
| 144 | 6 | chlorpropamide | 0.0001 | 6 | MCF7 | HG-U133A | EC2003092506AA | EC2003092502AA |
| 161 | 7a | verapamil | 0.00001 | 6 | MCF7 | HG-U133A | EC2003100103AA | EC2003100102AA |
| 164 | 7 | dexverapamil | 0.00001 | 6 | MCF7 | HG-U133A | EC2003100104AA | EC2003100102AA |
| 165 | 7 | exemestane | 0.00000001 | 6 | MCF7 | HG-U133A | EC2003100105AA | EC2003100102AA |
| 166 | 7 | rofecoxib | 0.00001 | 6 | MCF7 | HG-U133A | EC2003100106AA | EC2003100102AA |
| 167 | 8 | amitriptyline | 0.000001 | 6 | MCF7 | HG-U133A | EC2003100203AA | EC2003100202AA |
| 168 | 8 | sulindac | 0.0001 | 6 | MCF7 | HG-U133A | EC2003100205AA | EC2003100202AA |
| 169 | 8 | tacrolimus | 0.000001 | 6 | MCF7 | HG-U133A | EC2003100206AA | EC2003100202AA |
| 201 | 13 | 15-delta | 0.00001 | 6 | MCF7 | HG-U133A | EC2003103003AA | EC2003103002AA |
| | | prostaglandin | | | | | | |
| | | J2 | | | | | | |
| 202 | 13 | raloxifene | 0.0000001 | 6 | MCF7 | HG-U133A | EC2003103004AA | EC2003103002AA |
| 203 | 13 | nordihydro- | 0.000001 | 6 | MCF7 | HG-U133A | EC2003103005AA | EC2003103002AA |
| | | guaiaretic | | | | | | |
| | | acid | | | | | | |
| 204 | 13 | sulfasalazine | 0.0001 | 6 | MCF7 | HG-U133A | EC2003103006AA | EC2003103002AA |
| 205 | 16 | rofecoxib | 0.00001 | 6 | MCF7 | HG-U133A | EC2003110403AA | EC2003110402AA |
| 206 | 16 | celecoxib | 0.00001 | 6 | MCF7 | HG-U133A | EC2003110404AA | EC2003110402AA |
| 207 | 16 | LM-1685 | 0.00001 | 6 | MCF7 | HG-U133A | EC2003110405AA | EC2003110402AA |
| 208 | 16 | SC-58125 | 0.00001 | 6 | MCF7 | HG-U133A | EC2003110406AA | EC2003110402AA |
| 221 | 17 | 17-allylamino- | 0.0000001 | 6 | MCF7 | HG-U133A | EC2003120403AA | EC2003120402AA |
| | | geldanamycin | | | | | | |
| 222 | 17 | tomelukast | 0.000001 | 6 | MCF7 | HG-U133A | EC2003120404AA | EC2003120402AA |
| 223 | 17 | TTNPB | 0.0000001 | 6 | MCF7 | HG-U133A | EC2003120405AA | EC2003120402AA |
| 224 | 17 | tretinoin | 0.000001 | 6 | MCF7 | HG-U133A | EC2003120406AA | EC2003120402AA |
| 251 | 18 | rofecoxib | 0.00001 | 6 | MCF7 | HG-U133A | EC2003121103AA | EC2003121102AA |
| 252 | 18 | celecoxib | 0.00001 | 6 | MCF7 | HG-U133A | EC2003121104AA | EC2003121102AA |
| 253 | 18 | LM-1685 | 0.00001 | 6 | MCF7 | HG-U133A | EC2003121105AA | EC2003121102AA |
| 254 | 18 | SC-58125 | 0.00001 | 6 | MCF7 | HG-U133A | EC2003121106AA | EC2003121102AA |
| 255 | 19 | dexamethasone | 0.000001 | 6 | MCF7 | HG-U133A | EC2003121203AA | EC2003121202AA |
| 256 | 19 | rofecoxib | 0.00001 | 6 | MCF7 | HG-U133A | EC2003121204AA | EC2003121202AA |
| 258 | 19 | LY-294002 | 0.00001 | 6 | MCF7 | HG-U133A | EC2003121206AA | EC2003121202AA |
| 261 | 20 | ciclosporin | 0.000001 | 6 | MCF7 | HG-U133A | EC2004010803AA | EC2004010802AA |
| 262 | 20 | indometacin | 0.00002 | 6 | MCF7 | HG-U133A | EC2004010804AA | EC2004010802AA |
| 263 | 20 | clofibrate | 0.00015 | 6 | MCF7 | HG-U133A | EC2004010805AA | EC2004010802AA |
| 264 | 20 | MK-886 | 0.000001 | 6 | MCF7 | HG-U133A | EC2004010806AA | EC2004010802AA |
| 265 | 21 | prednisolone | 0.000001 | 6 | MCF7 | HG-U133A | EC2004010809AA | EC2004010808AA |
| 266 | 21 | thalidomide | 0.0001 | 6 | MCF7 | HG-U133A | EC2004010810AA | EC2004010808AA |
| 267 | 21 | genistein | 0.000001 | 6 | MCF7 | HG-U133A | EC2004010811AA | EC2004010808AA |
| 268 | 21 | genistein | 0.000001 | 6 | MCF7 | HG-U133A | EC2004010812AA | EC2004010808AA |
| 281 | 22a | fludrocortisone | 0.000001 | 6 | MCF7 | HG-U133A | EC2003122303AA | EC2003122302AA |
| 282 | 22a | fludrocortisone | 0.000001 | 6 | MCF7 | HG-U133A | EC2003122304AA | EC2003122302AA |
| 283 | 22a | quercetin | 0.000001 | 6 | MCF7 | HG-U133A | EC2003122305AA | EC2003122302AA |
| 284 | 22a | tacrolimus | 0.000001 | 6 | MCF7 | HG-U133A | EC2003122306AA | EC2003122302AA |
| 307 | 23 | sulindac | 0.00005 | 6 | MCF7 | HG-U133A | EC2003122309AA | EC2003122308AA |
| 308 | 23 | sulindac | 0.00005 | 6 | MCF7 | HG-U133A | EC2003122310AA | EC2003122308AA |
| | | sulfide | | | | | | |
| 309 | 23 | exisulind | 0.00005 | 6 | MCF7 | HG-U133A | EC2003122311AA | EC2003122308AA |
| 310 | 23 | fulvestrant | 0.00000001 | 6 | MCF7 | HG-U133A | EC2003122312AA | EC2003122308AA |
| 311 | 24 | monastrol | 0.0001 | 6 | MCF7 | HG-U133A | EC2003122315AA | EC2003122314AA |
| 312 | 24 | staurosporine | 0.000001 | 6 | MCF7 | HG-U133A | EC2004010817AA | EC2003122314AA |
| 313 | 24 | NU-1025 | 0.0001 | 6 | MCF7 | HG-U133A | EC2003122317AA | EC2003122314AA |
| 314 | 24 | exisulind | 0.00005 | 6 | MCF7 | HG-U133A | EC2003122318AA | EC2003122314AA |
| 315 | 25 | acetylsalicylic | 0.0001 | 6 | MCF7 | HG-U133A | EC2004011603AA | EC2004011602AA |
| | | acid | | | | | | |
| 316 | 25 | flufenamic acid | 0.00001 | 6 | MCF7 | HG-U133A | EC2004011604AA | EC2004011602AA |
| 317 | 25 | N- | 0.00001 | 6 | MCF7 | HG-U133A | EC2004011605AA | EC2004011602AA |
| | | phenylanthra- | | | | | | |
| | | nilic acid | | | | | | |
| 318 | 25 | LY-294002 | 0.00001 | 6 | MCF7 | HG-U133A | EC2004011606AA | EC2004011602AA |
| 325 | 26b | monorden | 0.0000001 | 6 | MCF7 | HG-U133A | EC2004012203AA | EC2004021313AA |
| 326 | 26b | sirolimus | 0.0000001 | 6 | MCF7 | HG-U133A | EC2004012204AA | EC2004021313AA |
| 327 | 26b | arachidonyl- | 0.00001 | 6 | MCF7 | HG-U133A | EC2004012205AA | EC2004021313AA |
| | | trifluoro- | | | | | | |
| | | methane | | | | | | |
| 328 | 26b | LY-294002 | 0.00001 | 6 | MCF7 | HG-U133A | EC2004012206AA | EC2004021313AA |
| 331 | 28 | trichostatin A | 0.0000001 | 6 | MCF7 | HG-U133A | EC2004012209AA | EC2004012208AA |
| 332 | 28 | trichostatin A | 0.0000001 | 6 | MCF7 | HG-U133A | EC2004012210AA | EC2004012208AA |
| 333 | 28 | diclofenac | 0.00001 | 6 | MCF7 | HG-U133A | EC2004012211AA | EC2004012208AA |
| 334 | 28 | mercaptopurine | 0.0001 | 6 | MCF7 | HG-U133A | EC2004012212AA | EC2004012208AA |
| 335 | 29 | nifedipine | 0.00001 | 6 | MCF7 | HG-U133A | EC2004010603AA | EC2004010602AA |
| 336 | 29 | nitrendipine | 0.00001 | 6 | MCF7 | HG-U133A | EC2004010604AA | EC2004010602AA |
| 337 | 29 | felodipine | 0.00001 | 6 | MCF7 | HG-U133A | EC2004010605AA | EC2004010602AA |
| 338 | 29 | azathioprine | 0.0001 | 6 | MCF7 | HG-U133A | EC2004010606AA | EC2004010602AA |
| 341 | 31 | sodium | 0.0001 | 6 | MCF7 | HG-U133A | EC2004021303AA | EC2004021302AA |
| | | phenylbutyrate | | | | | | |
| 342 | 31 | novobiocin | 0.0001 | 6 | MCF7 | HG-U133A | EC2004021304AA | EC2004021302AA |
| 343 | 31 | fasudil | 0.00001 | 6 | MCF7 | HG-U133A | EC2004021305AA | EC2004021302AA |
| 344 | 31 | 2-deoxy-D- | 0.01 | 6 | MCF7 | HG-U133A | EC2004021306AA | EC2004021302AA |
| | | glucose | | | | | | |
| 345 | 33 | valproic acid | 0.01 | 6 | MCF7 | HG-U133A | EC2004021309AA | EC2004021308AA |
| 346 | 33 | valproic acid | 0.002 | 6 | MCF7 | HG-U133A | EC2004021310AA | EC2004021308AA |
| 347 | 33 | valproic acid | 0.0005 | 6 | MCF7 | HG-U133A | EC2004021311AA | EC2004021308AA |
| 348 | 33 | valproic acid | 0.00005 | 6 | MCF7 | HG-U133A | EC2004021312AA | EC2004021308AA |
| 361 | 35 | LY-294002 | 0.00001 | 6 | HL60 | HG-U133A | EC2004030503AA | EC2004030502AA |
| 362 | 35 | sirolimus | 0.0000001 | 6 | HL60 | HG-U133A | EC2004030504AA | EC2004030502AA |
| 363 | 35 | sodium | 0.0001 | 6 | HL60 | HG-U133A | EC2004030505AA | EC2004030502AA |
| | | phenylbutyrate | | | | | | |
| 364 | 35 | trichostatin A | 0.0000001 | 6 | HL60 | HG-U133A | EC2004030506AA | EC2004030502AA |
| 365 | 36 | estradiol | 0.0000001 | 6 | MCF7 | HG-U133A | EC2004041403AA | EC2004041402AA |
| 366 | 36 | imatinib | 0.00001 | 6 | MCF7 | HG-U133A | EC2004041404AA | EC2004041402AA |
| 367 | 36 | fulvestrant | 0.000001 | 6 | MCF7 | HG-U133A | EC2004041405AA | EC2004041402AA |
| 368 | 36 | pirinixic acid | 0.0001 | 6 | MCF7 | HG-U133A | EC2004041406AA | EC2004041402AA |
| 369 | 37 | rosiglitazone | 0.00001 | 6 | HL60 | HG-U133A | EC2004032003AA | EC2004032002AA |
| 370 | 37 | troglitazone | 0.00001 | 6 | HL60 | HG-U133A | EC2004032004AA | EC2004032002AA |
| 371 | 37 | rofecoxib | 0.00001 | 6 | HL60 | HG-U133A | EC2004032005AA | EC2004032002AA |
| 373 | 38 | estradiol | 0.00000001 | 6 | ssMCF7 | HG-U133A | EC2004030509AA | EC2004030508AA |
| 374 | 38 | dexamethasone | 0.000001 | 6 | ssMCF7 | HG-U133A | EC2004030510AA | EC2004030508AA |
| 375 | 38 | tamoxifen | 0.000001 | 6 | ssMCF7 | HG-U133A | EC2004030511AA | EC2004030508AA |
| 376 | 38 | raloxifene | 0.0000001 | 6 | ssMCF7 | HG-U133A | EC2004030512AA | EC2004030508AA |
| 377 | 39 | celecoxib | 0.00001 | 6 | MCF7 | HG-U133A | EC2004030515AA | EC2004030514AA |
| 378 | 39 | tacrolimus | 0.000001 | 6 | MCF7 | HG-U133A | EC2004030516AA | EC2004030514AA |
| 379 | 39 | cobalt chloride | 0.0001 | 6 | MCF7 | HG-U133A | EC2004032007AA | EC2004030514AA |
| 380 | 39 | tamoxifen | 0.000001 | 6 | MCF7 | HG-U133A | EC2004030518AA | EC2004030514AA |
| 381 | 40 | 17-allylamino- | 0.000001 | 6 | MCF7 | HG-U133A | EC2004041409AA | EC2004041408AA |
| | | geldanamycin | | | | | | |
| 382 | 40 | genistein | 0.00001 | 6 | MCF7 | HG-U133A | EC2004041410AA | EC2004041408AA |
| 383 | 40 | cobalt chloride | 0.0001 | 6 | MCF7 | HG-U133A | EC2004041411AA | EC2004041408AA |
| 384 | 40 | tretinoin | 0.000001 | 6 | MCF7 | HG-U133A | EC2004041412AA | EC2004041408AA |
| 387 | 41 | estradiol | 0.00000001 | 6 | HL60 | HG-U133A | EC2004032010AA | EC2004032009AA |
| 388 | 41 | raloxifene | 0.0000001 | 6 | HL60 | HG-U133A | EC2004032011AA | EC2004032009AA |
| 389 | 41 | wortmannin | 0.00000001 | 6 | HL60 | HG-U133A | EC2004032012AA | EC2004032009AA |
| 390 | 41 | tretinoin | 0.000001 | 6 | HL60 | HG-U133A | EC2004032013AA | EC2004032009AA |
| 401 | 42 | LY-294002 | 0.00001 | 6 | ssMCF7 | HG-U133A | EC2004041603AA | EC2004041602AA |
| 402 | 42 | sirolimus | 0.0000001 | 6 | ssMCF7 | HG-U133A | EC2004041604AA | EC2004041602AA |
| 403 | 42 | alpha- | 0.00000001 | 6 | ssMCF7 | HG-U133A | EC2004041605AA | EC2004041602AA |
| | | estradiol | | | | | | |
| 404 | 42 | wortmannin | 0.00000001 | 6 | ssMCF7 | HG-U133A | EC2004041606AA | EC2004041602AA |
| 405 | 43 | tetraethylene- | 0.00001 | 6 | MCF7 | HG-U133A | EC2004041609AA | EC2004041608AA |
| | | pentamine | | | | | | |
| 406 | 43 | tetraethylene- | 0.0001 | 6 | MCF7 | HG-U133A | EC2004041610AA | EC2004041608AA |
| | | pentamine | | | | | | |
| 407 | 43 | sodium | 0.001 | 6 | MCF7 | HG-U133A | EC2004041611AA | EC2004041608AA |
| | | phenylbutyrate | | | | | | |
| 408 | 43 | sodium | 0.001 | 6 | MCF7 | HG-U133A | EC2004041612AA | EC2004041608AA |
| | | phenylbutyrate | | | | | | |
| 409 | 44 | valproic acid | 0.001 | 6 | HL60 | HG-U133A | EC2004042303AA | EC2004042302AA |
| 410 | 44 | valproic acid | 0.01 | 6 | HL60 | HG-U133A | EC2004042304AA | EC2004042302AA |
| 411 | 44 | sodium | 0.001 | 6 | HL60 | HG-U133A | EC2004042305AA | EC2004042302AA |
| | | phenylbutyrate | | | | | | |
| 412 | 44 | tetraethylene- | 0.0001 | 6 | HL60 | HG-U133A | EC2004042306AA | EC2004042302AA |
| | | pentamine | | | | | | |
| 413 | 45 | trichostatin A | 0.0000001 | 6 | ssMCF7 | HG-U133A | EC2004050703AA | EC2004050702AA |
| 414 | 45 | estradiol | 0.00000001 | 6 | ssMCF7 | HG-U133A | EC2004050704AA | EC2004050702AA |
| 415 | 45 | nordihydro- | 0.000001 | 6 | ssMCF7 | HG-U133A | EC2004050706AA | EC2004050702AA |
| | | guaiaretic | | | | | | |
| | | acid | | | | | | |
| 416 | 46 | clozapine | 0.00001 | 6 | MCF7 | HG-U133A | EC2004042309AA | EC2004042308AA |
| 417 | 46 | thioridazine | 0.00001 | 6 | MCF7 | HG-U133A | EC2004042310AA | EC2004042308AA |
| 418 | 46 | haloperidol | 0.00001 | 6 | MCF7 | HG-U133A | EC2004042311AA | EC2004042308AA |
| 419 | 46 | chlorpromazine | 0.00001 | 6 | MCF7 | HG-U133A | EC2004042312AA | EC2004042308AA |
| 421 | 53 | trifluoperazine | 0.00001 | 6 | MCF7 | HG-U133A | EC2004042315AA | EC2004042314AA |
| 422 | 53 | thioridazine | 0.000001 | 6 | MCF7 | HG-U133A | EC2004042316AA | EC2004042314AA |
| 423 | 53 | staurosporine | 0.0000001 | 6 | MCF7 | HG-U133A | EC2004042317AA | EC2004042314AA |
| 424 | 53 | LY-294002 | 0.0000001 | 6 | MCF7 | HG-U133A | EC2004042318AA | EC2004042314AA |
| 425 | 54 | staurosporine | 0.00000001 | 6 | MCF7 | HG-U133A | EC2004050709AA | EC2004050708AA |
| 426 | 54 | chlorpromazine | 0.000001 | 6 | MCF7 | HG-U133A | EC2004050710AA | EC2004050708AA |
| 427 | 54 | iloprost | 0.000001 | 6 | MCF7 | HG-U133A | EC2004050711AA | EC2004050703AA |
| 428 | 54 | 17-allylamino- | 0.000001 | 6 | MCF7 | HG-U133A | EC2004050712AA | EC2004050708AA |
| | | geldanamycin | | | | | | |
| 429 | 55 | LY-294002 | 0.00001 | 6 | PC3 | HG-U133A | EC2004052403AA | EC2004052402AA |
| 430 | 55 | rosiglitazone | 0.00001 | 6 | PC3 | HG-U133A | EC2004052404AA | EC2004052402AA |
| 431 | 55 | troglitazone | 0.00001 | 6 | PC3 | HG-U133A | EC2004052405AA | EC2004052402AA |
| 432 | 55 | 17-allylamino- | 0.000001 | 6 | PC3 | HG-U133A | EC2004052406AA | EC2004052402AA |
| | | geldanamycin | | | | | | |
| 433 | 56 | valproic acid | 0.001 | 6 | PC3 | HG-U133A | EC2004052409AA | EC2004052408AA |
| 434 | 56 | sodium | 0.001 | 6 | PC3 | HG-U133A | EC2004052410AA | EC2004052408AA |
| | | phenylbutyrate | | | | | | |
| 435 | 66 | novobiocin | 0.0001 | 6 | PC3 | HG-U133A | EC2004052411AA | EC2004052408AA |
| 436 | 56 | fasudil | 0.00001 | 6 | PC3 | HG-U133A | EC2004052412AA | EC2004052408AA |
| 437 | 58 | novobiocin | 0.0001 | 6 | MCF7 | HG-U133A | EC2004052415AA | EC2004052414AA |
| 438 | 58 | copper sulfate | 0.0001 | 6 | MCF7 | HG-U133A | EC2004052416AA | EC2004052414AA |
| 439 | 56 | oxamic acid | 0.01 | 6 | MCF7 | HG-U133A | EC2004052417AA | EC2004052414AA |
| 440 | 58 | W-13 | 0.00001 | 6 | MCF7 | HG-U133A | EC2004052418AA | EC2004052414AA |
| 441 | 59 | arachidonic | 0.00001 | 6 | MCF7 | HG-U133A | EC2004060203AA | CL2004060801AA |
| | | acid | | | | | | |
| 442 | 59 | oligomycin | 0.000001 | 6 | MCF7 | HG-U133A | EC2004060204AA | CL2004080801AA |
| 443 | 59 | arachidonic | 0.00001 | 6 | MCF7 | HG-U133A | EC2004060205AA | CL2004060801AA |
| | | acid | | | | | | |
| 444 | 59 | clofibrate | 0.0001 | 6 | MCF7 | HG-U133A | EC2004060206AA | CL2004060801AA |
| 445 | 60 | diclofenac | 0.00001 | 6 | PC3 | HG-U133A | EC2004060209AA | EC2004060208AA |
| 446 | 60 | 15-delta | 0.00001 | 6 | PC3 | HG-U133A | EC2004060210AA | EC2004060208AA |
| | | prostaglandin | | | | | | |
| | | J2 | | | | | | |
| 447 | 60 | tretinoin | 0.000001 | 6 | PC3 | HG-U133A | EC2004060211AA | EC2004060208AA |
| 448 | 60 | trichostatin A | 0.0000001 | 6 | PC3 | HG-U133A | EC2004060212AA | EC2004060208AA |
| 449 | 61 | monorden | 0.0000001 | 6 | PC3 | HG-U133A | EC2004060215AA | EC2004060214AA |
| 450 | 61 | 17-allylamino- | 0.000001 | 6 | PC3 | HG-U133A | EC2004060216AA | EC2004060214AA |
| | | geldanamycin | | | | | | |
| 451 | 61 | TTNPB | 0.0000001 | 6 | PC3 | HG-U133A | EC2004060217AA | EC2004060214AA |
| 452 | 61 | indometacin | 0.0001 | 6 | PC3 | HG-U133A | EC2004060218AA | EC2004060214AA |
| 453 | 62 | indometacin | 0.0001 | 6 | MCF7 | HG-U133A | EC2004070109AA | EC2004070108AA |
| 454 | 62 | cobalt chloride | 0.0001 | 6 | MCF7 | HG-U133A | EC2004070110AA | EC2004070108AA |
| 455 | 62 | prochlor- | 0.00001 | 6 | MCF7 | HG-U133A | EC2004070111AA | EC2004070108AA |
| | | perazine | | | | | | |
| 456 | 62 | quinpirole | 0.000001 | 6 | MCF7 | HG-U133A | EC2004070113AA | EC2004070108AA |
| 457 | 63 | tetraethylene- | 0.0001 | 6 | PC3 | HG-U133A | EC2004070103AA | EC2004070102AA |
| | | pentamine | | | | | | |
| 458 | 63 | valproic acid | 0.001 | 6 | PC3 | HG-U133A | EC2004070104AA | EC2004070102AA |
| 459 | 63 | copper sulfate | 0.0001 | 6 | PC3 | HG-U133A | EC2004070105AA | EC2004070102AA |
| 460 | 63 | deferoxamine | 0.0001 | 6 | PC3 | HG-U133A | EC2004070106AA | EC2004070102AA |
| 461 | 65 | LY-294002 | 0.00001 | 6 | PC3 | HG-U133A | CL2004060804AA | CL2004060803AA |
| 462 | 65 | troglitazone | 0.00001 | 6 | PC3 | HG-U133A | CL2004060805AA | CL2004060803AA |
| 463 | 65 | rofecoxib | 0.00001 | 6 | PC3 | HG-U133A | CL2004060806AA | CL2004060803AA |
| 464 | 65 | pirinixic acid | 0.0001 | 6 | PC3 | HG-U133A | CL2004060807AA | CL2004060803AA |
| 481 | 66 | pirinixic acid | 0.0001 | 6 | PC3 | HG-U133A | EC2004070115AA | EC2004070114AA |
| 482 | 66 | celecoxib | 0.00001 | 6 | PC3 | HG-U133A | EC2004070116AA | EC2004070114AA |
| 483 | 66 | imatinib | 0.00001 | 6 | PC3 | HG-U133A | EC2004070117AA | EC2004070114AA |
| 484 | 66 | monorden | 0.0000001 | 6 | PC3 | HG-U133A | EC2004070118AA | EC2004070114AA |
| 485 | 67 | deferoxamine | 0.0001 | 6 | MCF7 | HG-U133A | EC2004070121AA | EC2004070120AA |
| 488 | 67 | calmidazolium | 0.000005 | 6 | MCF7 | HG-U133A | EC2004070122AA | EC2004070120AA |
| 487 | 67 | pirinixic acid | 0.0001 | 6 | MCF7 | HG-U133A | EC2004070123AA | EC2004070120AA |
| 488 | 67 | iloprost | 0.000001 | 6 | MCF7 | HG-U133A | EC2004070124AA | EC2004070120AA |
| 489 | 68 | monorden | 0.0000001 | 6 | MCF7 | HG-U133A | EC2004071403AA | EC2004071402AA |
| 490 | 68 | fluphenazine | 0.00001 | 6 | MCF7 | HG-U133A | EC2004071404AA | EC2004071402AA |
| 491 | 68 | dopamine | 0.000001 | 6 | MCF7 | HG-U133A | EC2004071405AA | EC2004071402AA |
| 492 | 68 | haloperidol | 0.00001 | 6 | MCF7 | HG-U133A | EC200407140SAA | EC2004071402AA |
| 493 | 69 | monorden | 0.0000001 | 6 | SKMEL5 | HG-U133A | EC2004071409AA | EC2004071408AA |
| 494 | 69 | fluphenazine | 0.00001 | 6 | SKMEL5 | HG-U133A | EC2004071410AA | EC2004071408AA |
| 495 | 69 | pirinixic acid | 0.0001 | 6 | SKMEL5 | HG-U133A | EC2004071411AA | EC2004071408AA |
| 496 | 69 | iloprost | 0.000001 | 6 | SKMEL5 | HG-U133A | EC2004071412AA | EC2004071408AA |
| 497 | 70 | valproic acid | 0.001 | 6 | ssMCF7 | HG-U133A | EC2004071415AA | EC2004071414AA |
| 498 | 70 | tetraethylene- | 0.0001 | 6 | ssMCF7 | HG-U133A | EC2004071416AA | EC2004071414AA |
| | | pentamine | | | | | | |
| 499 | 70 | novobiocin | 0.0001 | 6 | ssMCF7 | HG-U133A | EC2004071417AA | EC2004071414AA |
| 500 | 70 | copper sulfate | 0.0001 | 6 | ssMCF7 | HG-U133A | EC2004071418AA | EC2004071414AA |
| 501 | 71 | LY-294002 | 0.00001 | 6 | SKMEL5 | HG-U133A | EC2004071421AA | EC2004071420AA |
| 502 | 71 | sodium | 0.0002 | 6 | SKMEL5 | HG-U133A | EC2004071422AA | EC2004071420AA |
| | | phenylbutyrate | | | | | | |
| 503 | 71 | indometacin | 0.0001 | 6 | SKMEL5 | HG-U133A | EC2004071423AA | EC2004071420AA |
| 504 | 71 | troglitazone | 0.00001 | 6 | SKMEL5 | HG-U133A | EC2004071424AA | EC2004071420AA |
| 505 | 73 | 17-allylamino- | 0.000001 | 6 | SKMEL5 | HG-U133A | EC2004073003AA | EC2004073002AA |
| | | geldanamycin | | | | | | |
| 506 | 73 | wortmannin | 0.00000001 | 6 | SKMEL5 | HG-U133A | EC2004073004AA | EC2004073002AA |
| 507 | 73 | SC-58125 | 0.00001 | 6 | SKMEL5 | HG-U133A | EC2004073005AA | EC2004073002AA |
| 508 | 73 | staurosporine | 0.00000001 | 6 | SKMEL5 | HG-U133A | EC2004073006AA | EC2004073002AA |
| 521 | 74 | 17-allylamino- | 0.000001 | 6 | ssMCF7 | HG-U133A | EC2004073009AA | EC2004073008AA |
| | | geldanamycin | | | | | | |
| 523 | 74 | fulvestrant | 0.000001 | 6 | ssMCF7 | HG-U133A | EC2004073011AA | EC2004073008AA |
| 524 | 74 | nordihydro- | 0.000001 | 6 | ssMCF7 | HG-U133A | EC2004073012AA | EC2004073008AA |
| | | guaiaretic | | | | | | |
| | | acid | | | | | | |
| 541 | 75 | gefitinib | 0.00001 | 6 | HL60 | HG-U133A | EC2004073015AA | EC2004073014AA |
| 542 | 75 | SC-58125 | 0.00001 | 6 | HL60 | HG-U133A | EC2004073016AA | EC2004073014AA |
| 543 | 75 | 1,5- | 0.0001 | 6 | HL60 | HG-U133A | EC2004073017AA | EC2004073014AA |
| | | isoquino- | | | | | | |
| | | linediol | | | | | | |
| 544 | 75 | monorden | 0.0000001 | 6 | HL60 | HG-U133A | EC2004073018AA | EC2004073014AA |
| 564 | 79 | 15-delta | 0.00001 | 6 | SKMEL5 | HG-U133A | EC2004111124AA | EC20D4111120AA |
| | | prostaglandin | | | | | | |
| | | J2 | | | | | | |
| 573 | 82 | deferoxamine | 0.0001 | 12 | MCF7 | HG-U133A | EC2004081915AA | EC2004081914AA |
| 574 | 82 | tetraethylene- | 0.0001 | 12 | MCF7 | HG-U133A | EC2004081916AA | EC2004081914AA |
| | | pentamine | | | | | | |
| 575 | 82 | copper sulfate | 0.0001 | 12 | MCF7 | HG-U133A | EC2004081917AA | EC2004081914AA |
| 576 | 82 | novobiocin | 0.0001 | 12 | MCF7 | HG-U133A | EC2004081918AA | EC2004081914AA |
| 578 | 86 | 4,5- | 0.00001 | 6 | PC3 | HG-U133A | EC2004111116AA | EC2004111114AA |
| | | dianilino- | | | | | | |
| | | phthalimide | | | | | | |
| 579 | 86 | fisetin | 0.00005 | 6 | PC3 | HG-U133A | EC2004111117AA | EC2004111114AA |
| 582 | 87 | butein | 0.00001 | 6 | PC3 | HG-U133A | EC2005020904AA | EC2005020902AA |
| 583 | 87 | HNMPA- | 0.000005 | 6 | PC3 | HG-U133A | EC2005020905AA | EC2005020902AA |
| | | (AM)3 | | | | | | |
| 584 | 87 | dimethyloxalyl- | 0.001 | 6 | PC3 | HG-U133A | EC2005020906AA | EC2005020902AA |
| | | glycine | | | | | | |
| 590 | 94 | 3-amino- | 0.01 | 6 | MCF7 | HG-U133A | EC2004112404AA | EC2004112402AA |
| | | benzamide | | | | | | |
| 591 | 94 | bucladesine | 0.00002 | 6 | MCF7 | HG-U133A | EC2004112405AA | EC2004112402AA |
| 592 | 94 | probucol | 0.00001 | 6 | MCF7 | HG-U133A | EC2004112406AA | EC2004112402AA |
| 593 | 95 | geldanamycin | 0.000001 | 6 | MCF7 | HG-U133A | EC2004111103AA | EC2004111102AA |
| 594 | 95 | arachidonyl- | 0.00001 | 6 | MCF7 | HG-U133A | EC2004111104AA | EC2004111102AA |
| | | trifluoro | | | | | | |
| | | methane | | | | | | |
| 595 | 95 | resveratrol | 0.00005 | 6 | MCF7 | HG-U133A | EC2004111105AA | EC2004111102AA |
| 596 | 95 | monastrol | 0.0001 | 6 | MCF7 | HG-U133A | EC2004111106AA | EC2004111102AA |
| 601 | 96 | MK-886 | 0.000001 | 6 | MCF7 | HG-U133A | EC2004102603AA | EC2004102602AA |
| 602 | 96 | ciclosporin | 0.000001 | 6 | MCF7 | HG-U133A | EC2004102604AA | EC2004102602AA |
| 603 | 96 | nifedipine | 0.00001 | 6 | MCF7 | HG-U133A | EC2004102605AA | EC2004102602AA |
| 604 | 96 | arachidonic | 0.00001 | 6 | MCF7 | HG-U133A | EC2004102606AA | EC2004102602AA |
| | | acid | | | | | | |
| 605 | 98 | monastrol | 0.00002 | 6 | MCF7 | HG-U133A | EC2004111109AA | EC2004111108AA |
| 606 | 98 | thalidomide | 0.0001 | 6 | MCF7 | HG-U133A | EC2004111110AA | EC2004111108AA |
| 607 | 98 | butein | 0.00001 | 6 | MCF7 | HG-U133A | EC2004111111AA | EC2004111108AA |
| 608 | 98 | NU-1025 | 0.0001 | 6 | MCF7 | HG-U133A | EC2004111112AA | EC2004111108AA |
| 609 | 101 | 5666823 | 0.0001 | 6 | MCF7 | HG-U133A | EC2004112409AA | EC2004112408AA |
| 610 | 101 | monastrol | 0.0001 | 6 | MCF7 | HG-U133A | EC2004112410AA | EC2004112408AA |
| 611 | 101 | geldanamycin | 0.000001 | 6 | MCF7 | HG-U133A | EC2004112411AA | EC2004112408AA |
| 612 | 101 | LM-1685 | 0.00001 | 6 | MCF7 | HG-U133A | EC2004112412AA | EC2004112408AA |
| 614 | 103 | monastrol | 0.00002 | 6 | MCF7 | HG-U133A | EC2004120710AA | EC2004120708AA |
| 621 | 107 | nocodazole | 0.000001 | 6 | MCF7 | HG-U133A | EC2004121703AA | EC2004121702AA |
| 622 | 107 | resveratrol | 0.00005 | 6 | MCF7 | HG-U133A | EC2004121704AA | EC2004121702AA |
| 624 | 107 | 4,5- | 0.00001 | 6 | MCF7 | HG-U133A | EC2004121706AA | EC2004121702AA |
| | | dianilino- | | | | | | |
| | | phthalimide | | | | | | |
| 627 | 108 | monastrol | 0.0001 | 6 | MCF7 | HG-U133A | EC2004121711AA | EC2004121708AA |
| 629 | 109 | valproic acid | 0.001 | 6 | SKMEL5 | HG-U133A | EC2005010703AA | EC2005010702AA |
| 630 | 109 | colchicine | 0.000001 | 6 | SKMEL5 | HG-U133A | EC2005010704AA | EC2005010702AA |
| 631 | 109 | benserazide | 0.00001 | 6 | SKMEL5 | HG-U133A | EC2005010705AA | EC2005010702AA |
| 632 | 109 | novobiocin | 0.0001 | 6 | SKMEL5 | HG-U133A | EC2005010706AA | EC2005010702AA |
| 638 | 111 | genistein | 0.00001 | 6 | MCF7 | HG-U133A | EC2005020910AA | EC2005020908AA |
| 639 | 111 | pentamidine | 0.0001 | 6 | MCF7 | HG-U133A | EC2005020911AA | EC2005020908AA |
| 640 | 111 | paclitaxel | 0.0000001 | 6 | MCF7 | HG-U133A | EC2005020912AA | EC2005020908AA |
| 641 | 112 | benserazide | 0.00001 | 6 | MCF7 | HG-U133A | EC2005020915AA | EC2005020914AA |
| 842 | 112 | tioguanine | 0.00001 | 6 | MCF7 | HG-U133A | EC2005020916AA | EC2005020914AA |
| 643 | 112 | W-13 | 0.00001 | 6 | MCF7 | HG-U133A | EC2005020917AA | EC2005020914AA |
| 644 | 112 | colchicine | 0.0000001 | 6 | MCF7 | HG-U133A | EC2005020918AA | EC2005020914AA |
| 661 | 90 | splitomicin | 0.00002 | 6 | PC3 | HG-U133A | EC2005030703AA | EC2005030702AA |
| 662 | 90 | resveratrol | 0.00005 | 6 | PC3 | HG-U133A | EC2005030704AA | EC2005030702AA |
| 663 | 90 | U0125 | 0.000001 | 6 | PC3 | HG-U133A | EC2005030705AA | EC2005030702AA |
| 664 | 90 | docosa- | 0.0001 | 6 | PC3 | HG-U133A | EC2005030706AA | EC2005030702AA |
| | | hexaenoic | | | | | | |
| | | acid ethyl | | | | | | |
| | | ester | | | | | | |
| 665 | 116 | estradiol | 0.00000001 | 6 | PC3 | HG-U133A | EC2005030715AA | EC2005030714AA |
| 666 | 116 | butirosin | 0.00001 | 6 | PC3 | HG-U133A | EC2005030716AA | EC2005030714AA |
| 667 | 116 | mercaptopurine | 0.00001 | 6 | PC3 | HG-U133A | EC2005030717AA | EC2005030714AA |
| 668 | 116 | monastrol | 0.0001 | 6 | PC3 | HG-U133A | EC2005030718AA | EC2005030714AA |
| 681 | 117 | monastrol | 0.0001 | 6 | MCF7 | HG-U133A | EC2005030721AA | EC2005030720AA |
| 702 | 110b | alpha- | 0.00000001 | 6 | PC3 | HG-U133A | EC2005033010AA | EC2005033008AA |
| | | estradiol | | | | | | |
| 703 | 110b | genistein | 0.00001 | 6 | PC3 | HG-U133A | EC2005033011AA | EC2005033008AA |
| 704 | 110b | fulvestrant | 0.000001 | 6 | PC3 | HG-U133A | EC2005033012AA | EC2005033008AA |
| 762 | 119 | alpha- | 0.00000001 | 6 | MCF7 | HG-U133A | EC2005061004AA | EC2005061002AA |
| | | estradiol | | | | | | |
| 782 | 120 | estradiol | 0.00000001 | 6 | HL60 | HG-U133A | EC2005081604AA | EC2005081602AA |
| 783 | 120 | colforsin | 0.00005 | 6 | HL60 | HG-U133A | EC2005081605AA | EC2005081602AA |
| 825 | 504 | rottlerin | 0.00001 | 6 | MCF7 | HT_HG-U133A | 5202764005790181113004.H02 | .H01.G02.E03.D04.B05.A06 |
| 826 | 504 | prazosin | 0.00001 | 6 | MCF7 | HT_HG-U133A | 5202764005790181113004.H03 | .H01.G02.E03.D04.B05.A06 |
| 828 | 504 | 5252917 | 0.000014 | 6 | MCF7 | HT_HG-U133A | 5202764005790181113004.H05 | .H01.G02.E03.D04.B05.A06 |
| 831 | 504 | 17-allylamino- | 0.000001 | 6 | MCF7 | HT_HG-U133A | 5202764005790181113004.G03 | .H01.G02.E03.D04.B05.A06 |
| | | geldanamycin | | | | | | |
| 832 | 504 | Y-27632 | 0.000003 | 6 | MCF7 | HT_HG-U133A | 5202764005790181113004.G04 | .H01.G02.E03.D04.B05.A06 |
| 833 | 504 | 5255229 | 0.000013 | 6 | MCF7 | HT_HG-U133A | 5202764005790181113004.G05 | .H01.G02.E03.D04.B05.A06 |
| 834 | 504 | 5211181 | 0.000012 | 6 | MCF7 | HT_HG-U133A | 5202764005790181113004.G06 | .H01.G02.E03.D04.B05.A06 |
| 835 | 504 | carbamazepine | 0.0000001 | 6 | MCF7 | HT_HG-U133A | 5202764005790181113004.F02 | .H01.G02.E03.D04.B05.A06 |
| 836 | 504 | monorden | 0.0000001 | 6 | MCF7 | HT_HG-U133A | 5202764005790181113004.F03 | .H01.G02.E03.D04.B05.A06 |
| 837 | 504 | blebbistatin | 0.000017 | 6 | MCF7 | HT_HG-U133A | 5202764005790181113004.F04 | .H01.G02.E03.D04.B05.A06 |
| 838 | 504 | 5248896 | 0.000011 | 6 | MCF7 | HT_HG-U133A | 5202764005790181113004.F05 | .H01.G02.E03.D04.B05.A06 |
| 839 | 504 | 5224221 | 0.000012 | 6 | MCF7 | HT_HG-U133A | 5202764005790181113004.F06 | .H01.G02.E03.D04.B05.A06 |
| 841 | 504 | resveratrol | 0.00001 | 6 | MCF7 | HT_HG-U133A | 5202764005790181113004.E02 | .H01.G02.E03.D04.B05.A06 |
| 842 | 504 | bucladesine | 0.000002 | 6 | MCF7 | HT_HG-U133A | 5202764005790181113004.E04 | .H01.G02.E03.D04.B05.A06 |
| 843 | 504 | 5279552 | 0.000022 | 6 | MCF7 | HT_HG-U133A | 5202764005790181113004.E05 | .H01.G02.E03.D04.B05.A06 |
| 844 | 504 | 5253409 | 0.000017 | 6 | MCF7 | HT_HG-U133A | 5202764005790181113004.E06 | .H01.G02.E03.D04.B05.A06 |
| 848 | 504 | felodipine | 0.00001 | 6 | MCF7 | HT_HG-U133A | 5202764005790181113004.D05 | .H01.G02.E03.D04.B05.A06 |
| 849 | 504 | tretinoin | 0.000001 | 6 | MCF7 | HT_HG-U133A | 5202764005790181113004.D06 | .H01.G02.E03.D04.B05.A06 |
| 862 | 504 | 5230742 | 0.000017 | 6 | MCF7 | HT_HG-U133A | 5202764005790181113004.C04 | .H01.G02.E03.D04.B05.A06 |
| 863 | 504 | oxaprozin | 0.0003 | 6 | MCF7 | HT_HG-U133A | 5202764005790181113004.C06 | .H01.G02.E03.D04.B05.A06 |
| 864 | 504 | geldanamycin | 0.000001 | 6 | MCF7 | HT_HG-U133A | 5202764005790181113004.C06 | .H01.G02.E03.D04.B05.A06 |
| 866 | 504 | ikarugamycin | 0.000002 | 6 | MCF7 | HT_HG-U133A | 5202764005790181113004.B02 | .H01.G02.E03.D04.B05.A06 |
| 868 | 504 | 5182598 | 0.000025 | 6 | MCF7 | HT_HG-U133A | 5202764005790181113004.B04 | .H01.G02.E03.D04.B05.A06 |
| 869 | 504 | wortmannin | 0.000001 | 6 | MCF7 | HT_HG-U133A | 5202764005790181113004.B06 | .H01.G02.E03.D04.B05.A06 |
| 870 | 504 | pyrvinium | 0.00000125 | 6 | MCF7 | HT_HG-U133A | 5202764005790181113004.A01 | .H01.G02.E03.D04.B05.A06 |
| 871 | 504 | ionomycin | 0.000002 | 6 | MCF7 | HT_HG-U133A | 5202764005790181113004.A02 | .H01.G02.E03.D04.B05.A06 |
| 873 | 504 | trichostatin A | 0.000001 | 6 | MCF7 | HT_HG-U133A | 5202764005790181113004.A04 | .H01.G02.E03.D04.B05.A06 |
| 874 | 504 | depudecin | 0.000001 | 6 | MCF7 | HT_HG-U133A | 5202764005790181113004.A05 | .H01.G02.E03.D04.B05.A06 |
| 881 | 505 | docosa- | 0.0001 | 6 | MCF7 | HT_HG-U133A | 5202764005790181113004.H08 | .H07.G08.E09.D10.B11.A12 |
| | | hexaenoic | | | | | | |
| | | acid ethyl | | | | | | |
| | | ester | | | | | | |
| 882 | 505 | ionomycin | 0.000002 | 6 | MCF7 | HT_HG-U133A | 5202764005790181113004.H09 | .H07.G08.E09.D10.B11.A12 |
| 885 | 505 | 5186223 | 0.000012 | 6 | MCF7 | HT_HG-U133A | 5202764005790181113004.H12 | .H07.G08.E09.D10.B11.A12 |
| 887 | 505 | celastrol | 0.0000025 | 6 | MCF7 | HT_HG-U133A | 5202764005790181113004.G09 | .H07.G08.E09.D10.B11.A12 |
| 889 | 505 | 5286656 | 0.00005 | 6 | MCF7 | HT_HG-U133A | 5202764005790181113004.G11 | .H07.G08.E09.D10.B11.A12 |
| 890 | 505 | 5149715 | 0.00001 | 6 | MCF7 | HT_HG-U133A | 5202764005790181113004.G12 | .H07.G08.E09.D10.B11.A12 |
| 892 | 505 | 5162773 | 0.000007 | 6 | MCF7 | HT_HG-U133A | 5202764005790181113004.F08 | .H07.G08.E09.D10.B11.A12 |
| 893 | 505 | pararosaniline | 0.00001 | 6 | MCF7 | HT_HG-U133A | 5202764005790181113004.F09 | .H07.G08.E09.D10.B11.A12 |
| 896 | 505 | 5152487 | 0.00001 | 6 | MCF7 | HT_HG-U133A | 5202764005790181113004.F12 | .H07.G08.E09.D10.B11.A12 |
| 898 | 505 | 5213008 | 0.000018 | 6 | MCF7 | HT_HG-U133A | 5202764005790181113004.E08 | .H07.G08.E09.D10.B11.A12 |
| 900 | 505 | 5186324 | 0.000002 | 6 | MCF7 | HT_HG-U133A | 5202764005790181113004.E11 | .H07.G08.E09.D10.B11.A12 |
| 901 | 505 | 5114445 | 0.00001 | 6 | MCF7 | HT_HG-U133A | 5202764005790181113004.E12 | .H07.G08.E09.D10.B11.A12 |
| 903 | 505 | 5151277 | 0.000014 | 6 | MCF7 | HT_HG-U133A | 5202764005790181113004.D08 | .H07.G08.E09.D10.B11.A12 |
| 904 | 505 | 5109870 | 0.000025 | 6 | MCF7 | HT_HG-U133A | 5202764005790181113004.D09 | .H07.G08.E09.D10.B11.A12 |
| 905 | 505 | clotrimazole | 0.00005 | 6 | MCF7 | HT_HG-U133A | 5202764005790181113004.D11 | .H07.G08.E09.D10.B11.A12 |
| 906 | 505 | calmidazolium | 0.000005 | 6 | MCF7 | HT_HG-U133A | 5202764005790181113004.D12 | .H07.G08.E09.D10.B11.A12 |
| 908 | 505 | 5140203 | 0.000015 | 6 | MCF7 | HT_HG-U133A | 5202764005790181113004.C08 | .H07.G08.E09.D10.B11.A12 |
| 909 | 505 | HC toxin | 0.0000001 | 6 | MCF7 | HT_HG-U133A | 5202764005790181113004.C09 | .H07.G08.E09.D10.B11.A12 |
| 910 | 505 | trifluoperazine | 0.00001 | 6 | MCF7 | HT_HG-U133A | 5202764005790181113004.C10 | .H07.G08.E09.D10.B11.A12 |
| 911 | 505 | wortmannin | 0.00000001 | 6 | MCF7 | HT_HG-U133A | 5202764005790181113004.C11 | .H07.G08.E09.D10.B11.A12 |
| 913 | 505 | colforsin | 0.00005 | 6 | MCF7 | HT_HG-U133A | 5202764005790181113004.B07 | .H07.G08.E09.D10.B11.A12 |
| 914 | 505 | rottlerin | 0.00001 | 6 | MCF7 | HT_HG-U133A | 5202764005790181113004.B08 | .H07.G08.E09.D10.B11.A12 |
| 915 | 505 | topiramate | 0.000003 | 6 | MCF7 | HT_HG-U133A | 5202764005790181113004.B09 | .H07.G08.E09.D10.B11.A12 |
| 916 | 505 | 17-allylamino- | 0.000001 | 6 | MCF7 | HT_HG-U133A | 5202764005790181113004.B10 | .H07.G08.E09.D10.B11.A12 |
| | | geldanamycin | | | | | | |
| 917 | 505 | quercetin | 0.000001 | 6 | MCF7 | HT_HG-U133A | 5202764005790181113004.B12 | .H07.G08.E09.D10.B11.A12 |
| 918 | 505 | ikarugamycin | 0.000002 | 6 | MCF7 | HT_HG-U133A | 5202764005790181113004.A07 | .H07.G08.E09.D10.B11.A12 |
| 919 | 505 | carbamazepine | 0.0000001 | 6 | MCF7 | HT_HG-U133A | 5202764005790181113004.A08 | .H07.G08.E09.D10.B11.A12 |
| 920 | 505 | decitabine | 0.0000001 | 6 | MCF7 | HT_HG-U133A | 5202764005790181113004.A09 | .H07.G08.E09.D10.B11.A12 |
| 921 | 505 | sirolimus | 0.0000001 | 6 | MCF7 | HT_HG-U133A | 5202764005790181113004.A10 | .H07.G08.E09.D10.B11.A12 |
| 922 | 505 | celecoxib | 0.00001 | 6 | MCF7 | HT_HG-U133A | 5202764005790181113004.A11 | .H07.G08.E09.D10.B11.A12 |
| 941 | 502 | rottlerin | 0.00001 | 6 | MCF7 | HT_HG-U133A | 5202764005791175120104.H02 | .H01.G02.E03.D04.B05.A06 |
| 942 | 502 | prazosin | 0.00001 | 6 | MCF7 | HT_HG-U133A | 5202764005791175120104.H03 | .H01.G02.E03.D04.B05.A06 |
| 944 | 502 | 5252917 | 0.000014 | 6 | MCF7 | HT_HG-U133A | 5202764005791175120104.H05 | .H01.G02.E03.D04.B05.A06 |
| 947 | 502 | 17-allylamino- | 0.000001 | 6 | MCF7 | HT_HG-U133A | 5202764005791175120104.G03 | .H01.G02.E03.D04.B05.A06 |
| | | geldanamycin | | | | | | |
| 948 | 502 | Y-27632 | 0.000003 | 6 | MCF7 | HT_HG-U133A | 5202764005791175120104.G04 | .H01.G02.E03.D04.B05.A06 |
| 949 | 502 | 5255229 | 0.000013 | 6 | MCF7 | HT_HG-U133A | 5202764005791175120104.G05 | .H01.G02.E03.D04.B05.A06 |
| 950 | 502 | 5211181 | 0.000012 | 6 | MCF7 | HT_HG-U133A | 5202764005791175120104.G06 | .H01.G02.E03.D04.B05.A06 |
| 952 | 502 | carbamazepine | 0.0000001 | 6 | MCF7 | HT_HG-U133A | 5202764005791175120104.F02 | .H01.G02.E03.D04.B05.A06 |
| 953 | 502 | monorden | 0.0000001 | 6 | MCF7 | HT_HG-U133A | 5202764005791175120104.F03 | .H01.G02.E03.D04.B05.A06 |
| 954 | 502 | blebbistatin | 0.000017 | 6 | MCF7 | HT_HG-U133A | 5202764005791175120104.F04 | .H01.G02.E03.D04.B05.A06 |
| 955 | 502 | 5248896 | 0.000011 | 6 | MCF7 | HT_HG-U133A | 5202764005791175120104.F05 | .H01.G02.E03.D04.B05.A06 |
| 956 | 502 | 5224221 | 0.000012 | 6 | MCF7 | HT_HG-U133A | 5202764005791175120104.F06 | .H01.G02.E03.D04.B05.A06 |
| 958 | 502 | resveratrol | 0.00001 | 6 | MCF7 | HT_HG-U133A | 5202764005791175120104.E02 | .H01.G02.E03.D04.B05.A06 |
| 959 | 502 | bucladesine | 0.000002 | 6 | MCF7 | HT_HG-U133A | 5202764005791175120104.E04 | .H01.G02.E03.D04.B05.A06 |
| 960 | 502 | 5279552 | 0.000022 | 6 | MCF7 | HT_HG-U133A | 5202764005791175120104.E05 | .H01.G02.E03.D04.B05.A06 |
| 961 | 502 | 5253409 | 0.000017 | 6 | MCF7 | HT_HG-U133A | 5202764005791175120104.E06 | .H01.G02.E03.D04.B05.A06 |
| 965 | 502 | felodipine | 0.00001 | 6 | MCF7 | HT_HG-U133A | 5202764005791175120104.D05 | .H01.G02.E03.D04.B05.A06 |
| 966 | 502 | tretinoin | 0.000001 | 6 | MCF7 | HT_HG-U133A | 5202764005791175120104.D06 | .H01.G02.E03.D04.B05.A06 |
| 970 | 502 | 5230742 | 0.000017 | 6 | MCF7 | HT_HG-U133A | 5202764005791175120104.C04 | .H01.G02.E03.D04.B05.A06 |
| 971 | 502 | oxaprozin | 0.0003 | 6 | MCF7 | HT_HG-U133A | 5202764005791175120104.C05 | .H01.G02.E03.D04.B05.A06 |
| 972 | 502 | geldanamycin | 0.000001 | 6 | MCF7 | HT_HG-U133A | 5202764005791175120104.C06 | .H01.G02.E03.D04.B05.A06 |
| 974 | 502 | ikarugamycin | 0.000002 | 6 | MCF7 | HT_HG-U133A | 5202764005791175120104.B02 | .H01.G02.E03.D04.B05.A06 |
| 976 | 502 | 5182598 | 0.000025 | 6 | MCF7 | HT_HG-U133A | 5202764005791175120104.B04 | .H01.G02.E03.D04.B05.A06 |
| 977 | 502 | wortmannin | 0.000001 | 6 | MCF7 | HT_HG-U133A | 5202764005791175120104.B06 | .H01.G02.E03.D04.B05.A06 |
| 978 | 502 | pyrvinium | 0.00000125 | 6 | MCF7 | HT_HG-U133A | 5202764005791175120104.A01 | .H01.G02.E03.D04.B05.A06 |
| 979 | 502 | ionomycin | 0.000002 | 6 | MCF7 | HT_HG-U133A | 5202764005791175120104.A02 | .H01.G02.E03.D04.B05.A06 |
| 981 | 502 | trichostatin A | 0.000001 | 6 | MCF7 | HT_HG-U133A | 5202764005791175120104.A04 | .H01.G02.E03.D04.B05.A06 |
| 982 | 502 | depudecin | 0.000001 | 6 | MCF7 | HT_HG-U133A | 5202764005791175120104.A05 | .H01.G02.E03.D04.B05.A06 |
| 983 | 506 | haloperidol | 0.00001 | 6 | MCF7 | HT_HG-U133A | 5202764005791175120104.H08 | .H07.G08.E09.D10.B11.A12 |
| 984 | 506 | acetylsalicylic | 0.0001 | 6 | MCF7 | HT_HG-U133A | 5202764005791175120104.H09 | .H07.G08.E09.D10.B11.A12 |
| | | acid | | | | | | |
| 985 | 506 | fulvestrant | 0.000001 | 6 | MCF7 | HT_HG-U133A | 5202764005791175120104.H10 | .H07.G08.E09.D10.B11.A12 |
| 986 | 506 | 17-allylamino- | 0.000001 | 6 | MCF7 | HT_HG-U133A | 5202764005791175120104.H11 | .H07.G08.E09.D10.B11.A12 |
| | | geldanamycin | | | | | | |
| 987 | 506 | sirolimus | 0.0000001 | 6 | MCF7 | HT_HG-U133A | 5202764005791175120104.H12 | .H07.G08.E09.D10.B11.A12 |
| 988 | 506 | estradiol | 0.0000001 | 6 | MCF7 | HT_HG-U133A | 5202764005791175120104.G07 | .H07.G08.E09.D10.B11.A12 |
| 989 | 506 | valproic acid | 0.001 | 6 | MCF7 | HT_HG-U133A | 5202764005791175120104.G09 | .H07.G08.E09.D10.B11.A12 |
| 990 | 506 | alpha-estradiol | 0.00000001 | 6 | MCF7 | HT_HG-U133A | 5202764005791175120104.G10 | .H07.G08.E09.D10.B11.A12 |
| 991 | 506 | tretinoin | 0.000001 | 6 | MCF7 | HT_HG-U133A | 5202764005791175120104.G11 | .H07.G08.E09.D10.B11.A12 |
| 992 | 506 | trichostatin A | 0.0000001 | 6 | MCF7 | HT_HG-U133A | 5202764005791175120104.G12 | .H07.G08.E09.D10.B11.A12 |
| 993 | 506 | 17- | 0.0000001 | 6 | MCF7 | HT_HG-U133A | 5202764005791175120104.F07 | .H07.G08.E09.D10.B11.A12 |
| | | dimethylamino- | | | | | | |
| | | geldanamycin | | | | | | |
| 994 | 506 | valproic acid | 0.0002 | 6 | MCF7 | HT_HG-U133A | 5202764005791175120104.F08 | .H07.G08.E09.D10.B11.A12 |
| 995 | 506 | prochlor- | 0.00001 | 6 | MCF7 | HT_HG-U133A | 5202764005791175120104.F09 | .H07.G08.E09.D10.B11.A12 |
| | | perazine | | | | | | |
| 996 | 506 | LY-294002 | 0.0000001 | 6 | MCF7 | HT_HG-U133A | 5202764005791175120104.F10 | .H07.G08.E09.D10.B11.A12 |
| 997 | 506 | chlorpromazine | 0.000001 | 6 | MCF7 | HT_HG-U133A | 5202764005791175120104.F11 | .H07.G08.E09.D10.B11.A12 |
| 998 | 506 | 17-allylamino- | 0.000001 | 6 | MCF7 | HT_HG-U133A | 5202764005791175120104.F12 | .H07.G08.E09.D10.B11.A12 |
| | | geldanamycin | | | | | | |
| 999 | 506 | monorden | 0.0000001 | 6 | MCF7 | HT_HG-U133A | 5202764005791175120104.E07 | .H07.G08.E09.D10.B11.A12 |
| 1000 | 506 | vorinostat | 0.00001 | 6 | MCF7 | HT_HG-U133A | 5202764005791175120104.E08 | .H07.G08.E09.D10.B11.A12 |
| 1001 | 506 | sirolimus | 0.0000001 | 6 | MCF7 | HT_HG-U133A | 5202764005791175120104.E10 | .H07.G08.E09.D10.B11.A12 |
| 1002 | 506 | valproic acid | 0.00005 | 6 | MCF7 | HT_HG-U133A | 5202764005791175120104.E11 | .H07.G08.E09.D10.B11.A12 |
| 1003 | 506 | nordihydro- | 0.000001 | 6 | MCF7 | HT_HG-U133A | 5202764005791175120104.E12 | .H07.G08.E09.D10.B11.A12 |
| | | guaiaretic | | | | | | |
| | | acid | | | | | | |
| 1004 | 506 | trifluoperazine | 0.00001 | 6 | MCF7 | HT_HG-U133A | 5202764005791175120104.D07 | .H07.G08.E09.D10.B11.A12 |
| 1005 | 506 | 17-allylamino- | 0.000001 | 6 | MCF7 | HT_HG-U133A | 5202764005791175120104.D08 | .H07.G08.E09.D10.B11.A12 |
| | | geldanamycin | | | | | | |
| 1006 | 506 | 17-allylamino- | 0.000001 | 6 | MCF7 | HT_HG-U133A | 5202764005791175120104.D09 | .H07.G08.E09.D10.B11.A12 |
| | | geldanamycin | | | | | | |
| 1007 | 506 | LY-294002 | 0.00001 | 6 | MCF7 | HT_HG-U133A | 5202764005791175120104.D11 | .H07.G08.E09.D10.B11.A12 |
| 1008 | 506 | geldanamycin | 0.000001 | 6 | MCF7 | HT_HG-U133A | 5202764005791175120104.D12 | .H07.G08.E09.D10.B11.A12 |
| 1009 | 506 | clozapine | 0.00001 | 6 | MCF7 | HT_HG-U133A | 5202764005791175120104.C07 | .H07.G08.E09.D10.B11.A12 |
| 1010 | 506 | thioridazine | 0.00001 | 6 | MCF7 | HT_HG-U133A | 5202764005791175120104.C08 | .H07.G08.E09.D10.B11.A12 |
| 1011 | 506 | 15-delta- | 0.00001 | 6 | MCF7 | HT_HG-U133A | 5202764005791175120104.C09 | .H07.G08.E09.D10.B11.A12 |
| | | prostaglandin | | | | | | |
| | | J2 | | | | | | |
| 1012 | 506 | troglitazone | 0.00001 | 6 | MCF7 | HT_HG-U133A | 5202764005791175120104.C10 | .H07.G08.E09.D10.B11.A12 |
| 1013 | 506 | rosiglitazone | 0.00001 | 6 | MCF7 | HT_HG-U133A | 5202764005791175120104.C11 | .H07.G08.E09.D10.B11.A12 |
| 1014 | 506 | trichostatin A | 0.000001 | 6 | MCF7 | HT_HG-U133A | 5202764005791175120104.C12 | .H07.G08.E09.D10.B11.A12 |
| 1015 | 506 | genistein | 0.00001 | 6 | MCF7 | HT_HG-U133A | 5202764005791175120104.B07 | .H07.G08.E09.D10.B11.A12 |
| 1016 | 506 | LY-294002 | 0.00001 | 6 | MCF7 | HT_HG-U133A | 5202764005791175120104.B08 | .H07.G08.E09.D10.B11.A12 |
| 1017 | 506 | fluphenazine | 0.00001 | 6 | MCF7 | HT_HG-U133A | 5202764005791175120104.B09 | .H07.G08.E09.D10.B11.A12 |
| 1019 | 506 | LY-294002 | 0.00001 | 6 | MCF7 | HT_HG-U133A | 5202764005791175120104.B12 | .H07.G08.E09.D10.B11.A12 |
| 1020 | 506 | valproic acid | 0.0005 | 6 | MCF7 | HT_HG-U133A | 5202764005791175120104.A07 | .H07.G08.E09.D10.B11.A12 |
| 1021 | 505 | estradiol | 0.00000001 | 6 | MCF7 | HT_HG-U133A | 5202764005791175120104.A08 | .H07.G08.E09.D10.B11.A12 |
| 1022 | 506 | sirolimus | 0.0000001 | 6 | MCF7 | HT_HG-U133A | 5202764005791175120104.A09 | .H07.G08.E09.D10.B11.A12 |
| 1023 | 506 | wortmannin | 0.00000001 | 6 | MCF7 | HT_HG-U133A | 5202764005791175120104.A10 | .H07.G08.E09.D10.B11.A12 |
| 1024 | 506 | haloperidol | 0.00001 | 6 | MCF7 | HT_HG-U133A | 5202764005791175120104.A11 | .H07.G08.E09.D10.B11.A12 |
| 1041 | 513 | haloperidol | 0.00001 | 6 | MCF7 | HT_HG-U133A | 5202764005789148112904.H02 | .H01.G02.E03.D04.B05.A06 |
| 1042 | 513 | acetylsalicylic | 0.0001 | 6 | MCF7 | HT_HG-U133A | 5202764005789148112904.H03 | .H01.G02.E03.D04.B05.A06 |
| | | acid | | | | | | |
| 1043 | 513 | fulvestrant | 0.000001 | 6 | MCF7 | HT_HG-U133A | 5202764005789148112904.H04 | .H01.G02.E03.D04.B05.A06 |
| 1044 | 513 | 17-allylamino- | 0.000001 | 6 | MCF7 | HT_HG-U133A | 5202764005789148112904.H05 | .H01.G02.E03.D04.B05.A06 |
| | | geldanamycin | | | | | | |
| 1045 | 513 | sirolimus | 0.0000001 | 6 | MCF7 | HT_HG-U133A | 5202764005789148112904.H06 | .H01.G02.E03.D04.B05.A08 |
| 1047 | 513 | valproic acid | 0.001 | 6 | MCF7 | HT_HG-U133A | 5202764005789148112904.G03 | .H01.G02.E03.D04.B05.A06 |
| 1048 | 513 | alpha-estradiol | 0.00000001 | 6 | MCF7 | HT_HG-U133A | 5202764005789148112904.G04 | .H01.G02.E03.D04.B05.A06 |
| 1049 | 513 | tretinoin | 0.000001 | 6 | MCF7 | HT_HG-U133A | 5202764005789148112904.G05 | .H01.G02.E03.D04.B05.A06 |
| 1050 | 513 | trichostatin A | 0.0000001 | 6 | MCF7 | HT_HG-U133A | 5202764005789148112904.G06 | .H01.G02.E03.D04.B05.A06 |
| 1051 | 513 | 17- | 0.0000001 | 6 | MCF7 | HT_HG-U133A | 5202764005789148112904.F01 | .H01.G02.E03.D04.B05.A06 |
| | | dimethylamino- | | | | | | |
| | | geldanamycin | | | | | | |
| 1053 | 513 | prochlor- | 0.00001 | 6 | MCF7 | HT_HG-U133A | 5202764005789148112904.F03 | .H01.G02.E03.D04.B05.A06 |
| | | perazine | | | | | | |
| 1054 | 513 | LY-294002 | 0.0000001 | 6 | MCF7 | HT_HG-U133A | 5202764005789148112904.F04 | .H01.G02.E03.D04.B05.A06 |
| 1055 | 513 | chlorpromazine | 0.000001 | 6 | MCF7 | HT_HG-U133A | 5202764005789148112904.F05 | .H01.G02.E03.D04.B05.A06 |
| 1056 | 513 | 17-allylamino- | 0.000001 | 6 | MCF7 | HT_HG-U133A | 5202764005789148112904.F06 | .H01.G02.E03.D04.B05.A06 |
| | | geldanamycin | | | | | | |
| 1057 | 513 | monorden | 0.0000001 | 6 | MCF7 | HT_HG-U133A | 5202764005789148112904.E01 | .H01.G02.E03.D04.B05.A06 |
| 1058 | 513 | vorinostat | 0.00001 | 6 | MCF7 | HT_HG-U133A | 5202764005789148112904.E02 | .H01.G02.E03.D04.B05.A06 |
| 1059 | 513 | sirolimus | 0.0000001 | 6 | MCF7 | HT_HG-U133A | 5202764005789148112904.E04 | .H01.G02.E03.D04.B05.A06 |
| 1060 | 513 | valproic acid | 0.00005 | 6 | MCF7 | HT_HG-U133A | 5202764005789148112904.E05 | .H01.G02.E03.D04.B05.A06 |
| 1061 | 513 | nordihydro- | 0.000001 | 6 | MCF7 | HT_HG-U133A | 5202764005789148112904.E06 | .H01.G02.E03.D04.B05.A06 |
| | | guaiaretic acid | | | | | | |
| 1063 | 513 | 17-allylamino- | 0.000001 | 6 | MCF7 | HT_HG-U133A | 5202764005789148112904.D02 | .H01.G02.E03.D04.B05.A06 |
| | | geldanamycin | | | | | | |
| 1064 | 513 | 17-allylamino- | 0.000001 | 6 | MCF7 | HT_HG-U133A | 5202764005789148112904.D03 | .H01.G02.E03.D04.B05.A06 |
| | | geldanamycin | | | | | | |
| 1065 | 513 | LY-294002 | 0.00001 | 6 | MCF7 | HT_HG-U133A | 5202764005789148112904.D05 | .H01.G02.E03.D04.B05.A06 |
| 1066 | 513 | geldanamycin | 0.000001 | 6 | MCF7 | HT_HG-U133A | 5202764005789148112904.D06 | .H01.G02.E03.D04.B05.A06 |
| 1068 | 513 | thioridazine | 0.00001 | 6 | MCF7 | HT_HG-U133A | 5202764005789148112904.C02 | .H01.G02.E03.D04.B05.A06 |
| 1069 | 513 | 15-delta | 0.00001 | 6 | MCF7 | HT_HG-U133A | 5202764005789148112904.C03 | .H01.G02.E03.D04.B05.A06 |
| | | prostaglandin | | | | | | |
| | | J2 | | | | | | |
| 1070 | 513 | troglitazone | 0.00001 | 6 | MCF7 | HT_HG-U133A | 5202764005789148112904.C04 | .H01.G02.E03.D04.B05.A06 |
| 1071 | 513 | rosiglitazone | 0.00001 | 6 | MCF7 | HT_HG-U133A | 5202764005789148112904.C05 | .H01.G02.E03.D04.B05.A06 |
| 1072 | 513 | trichostatin A | 0.000001 | 6 | MCF7 | HT_HG-U133A | 5202764005789148112904.C06 | .H01.G02.E03.D04.B05.A06 |
| 1073 | 513 | genistein | 0.00001 | 6 | MCF7 | HT_HG-U133A | 5202764005789148112904.B01 | .H01.G02.E03.D04.B05.A06 |
| 1074 | 513 | LY-294002 | 0.00001 | 6 | MCF7 | HT_HG-U133A | 5202764005789148112904.B02 | .H01.G02.E03.D04.B05.A06 |
| 1075 | 513 | fluphenazine | 0.00001 | 6 | MCF7 | HT_HG-U133A | 5202764005789148112904.B03 | .H01.G02.E03.D04.B05.A06 |
| 1076 | 513 | fulvestrant | 0.00000001 | 6 | MCF7 | HT_HG-U133A | 5202764005789148112904.B04 | .H01.G02.E03.D04.B05.A06 |
| 1077 | 513 | LY-294002 | 0.00001 | 6 | MCF7 | HT_HG-U133A | 5202764005789148112904.B06 | .H01.G02.E03.D04.B05.A06 |
| 1078 | 513 | valproic acid | 0.0005 | 6 | MCF7 | HT_HG-U133A | 5202764005789148112904.A01 | .H01.G02.E03.D04.B05.A06 |
| 1079 | 513 | estradiol | 0.00000001 | 6 | MCF7 | HT_HG-U133A | 5202764005789148112904.A02 | .H01.G02.E03.D04.B05.A06 |
| 1080 | 513 | sirolimus | 0.0000001 | 6 | MCF7 | HT_HG-U133A | 5202764005789148112904.A03 | .H01.G02.E03.D04.B05.A06 |
| 1081 | 513 | wortmannin | 0.00000001 | 6 | MCF7 | HT_HG-U133A | 5202764005789148112904.A04 | .H01.G02.E03.D04.B05.A06 |
| 1082 | 513 | haloperidol | 0.00001 | 6 | MCF7 | HT_HG-U133A | 5202764005789148112904.A05 | .H01.G02.E03.D04.B05.A06 |
| 1101 | 514 | (−)-catechin | 0.000011 | 6 | MCF7 | HT_HG-U133A | 5202764005789148112904.H08 | .H07.G08.E09.D10.B11.A12 |
| 1103 | 514 | demecolcine | 0.0000117 | 6 | MCF7 | HT_HG-U133A | 5202764005789148112904.H10 | .H07.G08.E09.D10.B11.A12 |
| 1105 | 514 | monensin | 0.0000109 | 6 | MCF7 | HT_HG-U133A | 5202764005789148112904.G07 | .H07.G08.E09.D10.B11.A12 |
| 1108 | 514 | 12,13-EODE | 0.0000002 | 6 | MCF7 | HT_HG-U133A | 5202764005789148112904.G11 | .H07.G08.E09.D10.B11.A12 |
| 1109 | 514 | 3-hydroxy-DL- | 0.0000092 | 6 | MCF7 | HT_HG-U133A | 5202764005789148112904.G12 | .H07.G08.E09.D10.B11.A12 |
| | | kynurenine | | | | | | |
| 1112 | 514 | trichostatin A | 0.0000001 | 6 | MCF7 | HT_HG-U133A | 5202764005789148112904.F09 | .H07.G08.E09.D10.B11.A12 |
| 1113 | 514 | doxycycline | 0.0000144 | 6 | MCF7 | HT_HG-U133A | 5202764005789148112904.F10 | .H07.G08.E09.D10.B11.A12 |
| 1114 | 514 | tyrphostin | 0.0000252 | 6 | MCF7 | HT_HG-U133A | 5202764005789148112904.F11 | .H07.G08.E09.D10.B11.A12 |
| | | AG-825 | | | | | | |
| 1115 | 514 | phenan- | 0.0000512 | 6 | MCF7 | HT_HG-U133A | 5202764005789148112904.F12 | .H07.G08.E09.D10.B11.A12 |
| | | thridinone | | | | | | |
| 1119 | 514 | yohimbine | 0.0000229 | 6 | MCF7 | HT_HG-U133A | 5202764005789148112904.E11 | .H07.G08.E09.D10.B11.A12 |
| 1121 | 514 | DL-PPMP | 0.000002 | 6 | MCF7 | HT_HG-U133A | 5202764005789148112904.D07 | .H07.G08.E09.D10.B11.A12 |
| 1122 | 514 | cytochalasin B | 0.0000208 | 6 | MCF7 | HT_HG-U133A | 5202764005789148112904.D08 | .H07.G08.E09.D10.B11.A12 |
| 1132 | 514 | BW-B70C | 0.0000316 | 6 | MCF7 | HT_HG-U133A | 5202764005789148112904.B07 | .H07.G08.E09.D10.B11.A12 |
| 1135 | 514 | minocycline | 0.0000105 | 6 | MCF7 | HT_HG-U133A | 5202764005789148112904.B10 | .H07.G08.E09.D10.B11.A12 |
| 1138 | 514 | phentolamine | 0.0000115 | 6 | MCF7 | HT_HG-U133A | 5202764005789148112904.A08 | .H07.G08.E09.D10.B11.A12 |
| 1140 | 514 | MG-132 | 0.000021 | 6 | MCF7 | HT_HG-U133A | 5202764005789148112904.A10 | .H07.G08.E09.D10.B11.A12 |
| 1141 | 514 | tyrphostin | 0.0000316 | 6 | MCF7 | HT_HG-U133A | 5202764005789148112904.A11 | .H07.G08.E09.D10.B11.A12 |
| | | AG-1478 | | | | | | |

¹“ssMCF7” indicates MCF7 cells cultured in phenol red free medium supplemented with charcoal-stripped serum
²“HG-U133A” represents Affymetrix part number 510681; “HT_HG-133A” represents Asymetrix part number 520276
³instances generated with HT_HG-U133A use six vehicle scans; the identifiers for these are constructed by appending each of the six extensions (eg “.H01”)
in this field to the twenty-two character number preceding the period (ie “.”) from the corresponding perturbation_scan_id

### Western Blotting

Western blotting was carried out as described (Ebert et al., 2005). The following antibodies were used: AR N-20 (1:250, sc-816, Santa Cruz), EGFR (1:1000, CST2232, Cell Signaling), ABL (1:1000, CST2862, Cell Signaling), phospho-tyrosine 4G10 for P-BCR-ABL1 (05-321, Upstate), FLT3/FLK2 S-18 (1:1000, sc-480, Santa Cruz), HSP90α (1:250, Stressgen, SPS-771F), HSP90 (1:5000, Abeam), CSK H-75 (1:250, Santa Cruz, sc-13074×), DDR1 H-126 (1:250, Santa Cruz, se-8988×), hHSP90 H9010, Hop F5, and p23 JJ3, tubulin (1:5000, Abcam, ab6046), and actin (1:5000, Abeam, ab8227-50).

### HSP90 ATP-Binding Assay

The ATP-binding assay was similar to that in previous reports (Bali et al., 2005, Soti et al., 2002). LNCaP and K562 cells were treated with celastrol and gedunin for 24 hr and then lysed in TNESV buffer (50 mM Tris, 2 mM EDTA, 100 nM NaCl, 1 mM activated sodium orthovanadate, 25 mM NaF, 1% Triton X-100 [pH 7.5]) for 30 min at 4° C. Lysates were spun for 30 min at 12,000 rpm at 4° C. Protein (200 μg) was incubated with conditioned γ-ATP-polyacrylamide resin (Novagen) in incubation buffer (10 mM Tris-HCl, 50 mM KCl, 5 mM MgCl₂, 20 mM Na₂MoO₄, 0.01% Nonidet P-40) overnight at 4° C., rotating. The resin was then washed four times with incubation buffer. Bound proteins were isolated by boiling with SDS buffer. HSP90 coimmunoprecipitation

SKBR-3 cells were treated with vehicle, celastrol (2.5 μM, 12 hr), and PU24FCI (20 μM, 24 hr) (Vilenchik et al., 2004). Cells were lysed in 20 mM Tris HCl (pH 7.4), 25 mM NaCl, 2 mM DDT, 20 mM Na₂MoO₄, 0.1% NP-40, and protein inhibitors. Lysates were incubated for 2 hr at 4° C., rotating, and then centrifuged at 13,000×g for 10 min. Protein (500 μg) was incubated with H9010 anti-HSP90 antibody for 1 hr at 4° C., rotating. Protein G agarose (30 μl; Upstate) was added to each sample, and samples were then incubated for 1 hr at 4° C., rotating. The beads were washed five times with 1 ml lysis buffer. Bound proteins were isolated by boiling in sample buffer. The levels of HSP90 and coimmunoprecipitating proteins were analyzed by western blot.

### Geldanamycin Competition Assay

The geldanamycin competition assay was performed as described (He et al., 2006, Kim et al., 2004), except that Cy3B-geldanamycin rather than BODIPY-geldanamycin was used as described herein.

### Gene Expression Signature Analysis

The androgen signaling signature was developed from existing Affymetrix U133A microarray data from LNCaP cells treated with 0.1 nM R1881 over a 24 h time course (Febbo et al., 2005). The MAS5-processed data was filtered and thresholded (min. fold difference=2.5, min absolute difference=50, floor=5, ceiling=16000). Class Neighbors analysis (GenePattern, http://www.broad.mit.edu/cancer/software/genepattern/) was used to identify genes that are differentially expressed at 12 h and 24 h of R1881 treatment relative to vehicle treatment by the signal-to-noise metric (Golub et al. (1999). The marker genes then filtered for induced expression of >100 and tested by GE-HTS androgen signaling assay. The top 27 genes with differential expression between androgen-treated and -deprived states by median SNR were chosen as the GE-HTS signature. Two normalization controls, SRP72 and KIAA0676, were selected from genes with moderate expression levels that varied little over the R1881 time course.

For the celastrol and gedunin signatures, RMA-processed data was filtered and thresholded (min fold change=2, min absolute change=50, floor=10, ceiling=16000). Comparative marker selection (GenePattern) was used to identify markers that distinguished celastrol- and/or gedunin-treated samples from vehicle-treated samples by the median SNR. The top fifty markers that increased and decreased relative to vehicle treated controls were used as the celastrol, gedunin, or joint celastrol/gedunin signatures.

### GE-HTS Androgen Signaling Signature Assay

Cell treatment. LNCaP cells were grown for 2d in RPMI 1640 media containing 10% charcoal-stripped FBS and then treated with 1 nM R1881 plus any compound of interest for 24 hours.

Ligation-mediated amplification. Cells were lysed by direct addition of lysis buffer (Turbo Capture 384 mRNA kit, Qiagen). Poly(A)⁺ RNA was isolated from the lysate by hybridization to dT₂₀-conjugated multiwell plates at room temperature (Qiagen) and reverse transcribed (MMLV, Promega). Probe pairs were annealed to the resulting cDNA by incubating at 95° C. for 2 min, followed by 50° C. for 60 min; the probe pairs consist of sequence complementary to 40 bp region of each transcript in the signature and flanked by a barcode sequence and universal T3/T7 primer sites probe sequences (listed in Table 3). Unbound probes were spun out of the plate, and the annealed probe pairs were ligated together (Taq ligase, NEB). The resulting ligation products were amplified by PCR for 29 cycles using T3 and biotylated-T7 probes (HotStarTaq, Qiagen). All steps were carried out in 5 ul volumes, except for the initial RNA hybridization, which used a 25 ul lysate volume. Before each step the prior reaction mix was spun out of the plate.

Luminex-bead based detection. To quantify the amplified cDNA products, the PCR product was then hybridized to a set of uniquely-colored, barcode-conjugated polystyrene beads (Luminex), where each bead color corresponds to a different barcode and gene. Hybridization was carried out at 45° C. for 60 min in TMAC (2.4M tetramethylammonium chloride, 0.08% sarkosyl, 42 mM Tris, and 3.4 mM EDTA). Streptavidin-phycoerythrin (101 μg/ml, SAPE, Molecular Probes) was added to detect the biotinylated PCR product. The beads were incubated for 10 min at 45° C. and then washed in TMAC. The SAPE fluorescence and color of each bead were measured by two-laser FACS (Lumiriex). The median SAPE intensity for a given bead color was used as the raw expression level of the corresponding gene. For each well, the raw GE-HTS expression levels are normalized to the control gene level(s).

### GE-HTS Screening

NINDS, Biomol, and SpecPlus libraries (//www.broad.mit.edu/chembio/platform/screening/compound_libraries/index.htm) were screened using GE-HTS androgen signaling and viability assays. After 2d androgen deprivation, LNCaP cells were treated with compounds (˜20 μM) or vehicle (DMSO) plus 1 nM R1881 for 24 h for the GEHTS screen and for 3d for the viability screen. Control wells were treated with (a) 1 nM R1881 plus vehicle, (b) 1 nM R1881 plus 10 μM casodex, or (c) vehicle alone.

### GE-HTS Analysis

Data from the screen was analyzed with a pipeline that contained algorithms directed to identify and prioritize likely modulators of the prostate androgen signature. Raw GE-HTS expression levels were filtered to remove wells containing SRP72 signal less than a standard deviation below the mean in wells containing media only. They were then were normalized to the SRP72 control gene level (NINDS) or mean of the SRP72 and KIAA0676 levels (Biomol, SpecPlus). The signal was scaled between plates by dividing each genes value in each well by the median value of that gene in the value for the vehicle control wells. Compounds were scored by simple weighted and unweighted ‘summed score’ metrics, a KNN classifier, and a naive Bayes classifier to identify candidate modulators of the prostate androgen signature. Implementation of these metrics are detailed belows. Heat maps of screen data were generated using data normalized between libraries by the mean SRP72 value for the 1 nM R1881 vehicle controls.

### Weighted Summed Score

The weighted summed scored metric combines the gene expression ratios of the signature by simply forming a weighted sum:

$$S = {\sum }_{i} {W}_{l} {x}_{l}$$

where Wᵢ represents the weight for gene expression ratio x, for gene i. The weight Wᵢ and its sign were determined by the strength of the gene ratio for separating the screen's positive and negative controls. The signal-to-noise ratio between the DMSO treated cells and the 1 nM R1881 treated cells was used to define the weight Wᵢ. Signal-to-noise ratio is defined by:

$${W}_{i} = \frac{{μ}_{i 1} - {μ}_{i 2}}{{σ}_{i 1} + {σ}_{i 2}}$$

where μᵢₗ represents the mean expression of samples from class 1 for feature i and σᵢₗ represents the standard deviation of class 1 for feature i (Golub et al., “Molecular Classification of Cancer: Class Discovery and Class Prediction by Gene Expression Monitoring,” Science, 1999). This approach, although simple, nicely complements the other methods of classification because it does not constrain the candidate compounds to closely follow the specific pattern of expression for the control samples and allows some variability among the individual genes. Composite scores were formed by finding the total of the weighted summed score from the three replicates.

Each compound's weighted summed score was assigned a probability that the compound caused the cells to have an expression signature like those for the DMSO treated control wells. The calculation of the probability was based upon finding the Bayesian probability of the weighted summed score using normal distributions to model the two classes of controls:

$$p ( C = c \vert X = x ) = \frac{p ( C = c ) p ( X = x \vert C = c )}{p ( X = x )} , where$$

$$p ( X = x \vert C = c ) = N ( x ; {μ}_{c} , {σ}_{c} )$$

where N(x; μ_{σ}, σ_{c}) was the probability density function for a normal (or Gaussian) distribution with mean p and standard deviation σ_{c} (Duda, R. O. and Hart, P. E., Pattern Classification and Scene Analysis, New York: John Wiley, 1973.). The parameters for the Gaussian distribution were trained on the positive and negative controls and p(C=c) was the a priori probability of class c controls (in this case, we assumed the positive and negative controls have equal a priori probabilities).

Composite probabilities were found by taking the product of the probabilities for the three replicates (but leaving out filtered replicates) and renormalizing the probabilities to ensure that the probability that the compound is a positive control and the probability that the compound is a negative control sum to one. Compounds were ranked for follow-up according to the probability that they looked like a positive control (DMSO treated).

### KNN Classifier

The k-nearest-neighbor (KNN) classifier that classifies samples by assigning them the label most frequently represented among the k nearest samples was also used to identify possible hits (Duda, R. O. and Hart, P. E., Pattern Classification and Scene Analysis, New York: John Wiley, 1973.). A KNN predictor was trained using the 1 nM R1881 treated and DMSO treated control samples and the compound treated wells were tested using k=5 with a Pearson correlation for the distance metric with weights for the neighbors based upon the Pearson distance. A modified version of KNN was used where the genes were weighted based upon the signal-to-noise ratio in the control samples.

Naïve Bayes Classifier

Naïve Bayes classifier was also used to evaluate the expression signatures for the compounds. The Natve Bayes classifier is based upon the Bayes probability rule and naively assumes that the features are independent within each class. The independence assumption greatly simplifies the calculation of the class probabilities and has been shown to work well even in cases where the features have significant dependencies. The probabilities are calculated as follows:

$$p ( C = c \vert X = x ) = \frac{p ( C = c ) p ( X = x \vert C = c )}{p ( X = x )} , where$$

$$p ( X = x \vert C = c ) = {\prod }_{i} p ( {X}_{i} = {x}_{i} \vert C = c )$$

where for continuous values like the gene expression ratios p(Xi=xi|C=c) can be either a Gaussian (i.e., normal) distribution or a kernel distribution formed out of a mixture of Gaussians (John, G. H. and Langley P. “Estimating Continuous Distributions in Bayesian Classifiers,” Proc. of the II^{t″} Conf on Uncertainty in Artificial Intelligence, 1995.). In either case, the parameters for the distribution for each class c and each feature i are trained using the controls for the screen. The first screen used the Gaussian in the Naïve Bayes estimator while the second screen used the kernel estimator. The overall probability for each compound is found by multiplying the probabilities for the individual replicates (leaving out filtered replicates) and renormalizing the probabilities so the two classes to sum to one. Compounds were ranked for follow-up according to the probability that they looked like a positive control (DMSO treated).

### Hierarchical Clustering

For hierarchical clustering, a 169 probe set of androgen-regulated genes was defined (p<0.05 based on 1000 permutations of signal-to-noise ratio after thresholding and filtering) using an independent data set (Febbo et al., 2005). We median centered these genes and arrays twice (median polished) and then normalized the genes. Cluster and TreeView software was used to perform average linkage hierarchical clustering and weighted centered correlation within the space of androgen-regulated genes (Eisen et al., 1998)

### Connectivity Map Analysis for Drug Activity

The current version of The Connectivity Map dataset (build01) contains genome-wide expression data for 453 treatment and vehicle control pairs, representing 164 distinct small molecules. Cell treatments were predominantly carried out in the MCF7 cell line for 6 h as detailed in Table 4. Affymetrix profiling was then carried out as described (Lamb et al., 2006). Enrichment of the induced- and repressed genes of a signature within each Connectivity Map treatment profile were estimated with a metric based on the Kolmogorov-Smirnov statistic as described (Lamb et al., 2003) and combined to produce a connectivity score. The connectivity score was set to zero (‘null’) where the enrichment scores for the up- and down-regulated gene sets were of the same sign. Raw expression data are available at www.broad.mit.edu/cmap and NCBI's Gene Expression Omnibus (GEO, www.ncbi.nlm.nih.gov/geo/, series accession number GSE5258). Connectivity Map analysis tools are also available at www.broad.mit.edu/cmap.

### Hsp90 Competition Assay

Measurements were taken in black 96-well microtiter plates (Coming #3650). The assay buffer (HFB) contained 20 mM HEPES (K) pH 7.3, 50 mM KCl, 5 mM MgCl₂, 20 mM Na₂MoO₄, 0.01% NP40. Before each use, 0.1 mg/mL bovine gamma globulin (BGG) (Panvera Corporation, Madison, Wis.) and 2 mM DTT (Fisher Biotech, Fair Lawn, N.J.) were freshly added. GM-cy3B was synthesized as previously reported and was dissolved in DMSO to form 10 μM solutions. Cell lysates were prepared rupturing cellular membranes by freezing at −70° C. and dissolving the cellular extract in HFB with added protease and phosphotase inhibitors. Saturation curves were recorded in which GM-cy3B (3 nM) was treated with increasing amounts of cellular lysates. The amount of lysate that resulted in polarization (mP) readings corresponding to 20 nM recombinant Hsp90α was chosen for the competition study. For the competition studies, each 96-well contained 3 nM fluorescent GM, 20 nM Hsp90a (Stressgen#SPP776) or cellular lysate (amounts as determined above and normalized to total Hsp90 as determined by Western blot analysis using as standard recombinant Hsp90α (Stressgen#SPP-776) and tested inhibitor (initial stock in DMSO) in a final volume of 100 μL. The plate was left on a shaker at 4° C. for 24 h and the FP values in mP were recorded in an Analyst GT instrument (Molecular Devices, Sunnyvale, Calif.). EC₅₀ values were determined as the competitor concentrations at which 50% of the fluorescent GM was displaced.

### REFERENCES

Bagatell, R., Beliakoff, J., David, C. L., Marron, M. T., and Whitesell, L. (2005). Hsp90 inhibitors deplete key anti-apoptotic proteins in pediatric solid tumor cells and demonstrate synergistic anticancer activity with cisplatin. Int J Cancer 113, 179-188.
Bali, P., Pranpat, M., Bradner, J., Balasis, M., Fiskus, W., Guo, F., Rocha, K., Kumaraswamy, S., Boyapalle, S., Atadja, P., et al. (2005). Inhibition of histone deacetylase 6 acetylates and disrupts the chaperone function of heat shock protein 90: a novel basis for antileukemia activity of histone deacetylase inhibitors. J Biol Chem 280, 26729-26734.
Chen, C. D., Welsbie, D. S., Tran, C., Baek, S. H., Chen, R., Vessella, R., Rosenfeld, M. G., and Sawyers, C. L. (2004). Molecular determinants of resistance to antiandrogen therapy. Nat Med 10, 33-39.
di Bernardo, D., Thompson, M. J., Gardner, T. S., Chobot, S. E., Eastwood, E. L., Wojtovich, A. P., Elliott, S. J., Schaus, S. E., and Collins, J. J. (2005). Chemogenomic profiling on a genome-wide scale using reverse-engineered gene networks. Nat Biotechnol 23, 377-383.
Ebert, B. L., Lee, M. M., Pretz, J. L., Subramanian, A., Mak, R., Golub, T. R., and Sieff, C. A. (2005). An RNA interference model of RPS19 deficiency in Diamond-Blackfan anemia recapitulates defective hematopoiesis and rescue by dexamethasone: identification of dexamethasone-responsive genes by microarray. Blood 105, 4620-4626.
Febbo, P. G., Lowenberg, M., Thorner, A. R., Brown, M., Loda, M., and Golub, T. R. (2005). Androgen mediated regulation and functional implications of fkbp51 expression in prostate cancer. J Urol 173, 1772-1777.
Feldman, B. J., and Feldman, D. (2001). The development of androgen-independent prostate cancer. Nat Rev Cancer 1, 34-45.
Felts, S. J., and Toft, D. O. (2003). p23, a simple protein with complex activities. Cell Stress Chaperones 8, 108-113.
Ficker, E., Dennis, A. T., Wang, L., and Brown, A. M. (2003). Role of the cytosolic chaperones Hsp70 and Hsp90 in maturation of the cardiac potassium channel HERG. Circ Res 92, e87-100.
Figueiredo, J. N., Raz, B., and Sequin, U. (1998). Novel quinone methides from Salacia kraussii with in vitro antimalarial activity. J Nat Prod 61, 718-723.
Gardner, T. S., di Bemardo, D., Lorenz, D., and Collins, J. J. (2003). Inferring genetic networks and identifying compound mode of action via expression profiling. Science 301, 102-105.
Georget, V., Terouanne, B., Nicolas, J. C., and Sultan, C. (2002). Mechanism of antiandrogen action: key role of hsp90 in conformational change and transcriptional activity of the androgen receptor. Biochemistry 41, 11824-11831.
Hahn, W. C., Counter, C. M., Lundberg, A. S., Beijersbergen, R. L., Brooks, M. W., and Weinberg, R. A. (1999). Creation of human tumour cells with defined genetic elements. Nature 400, 464-468.
He, H., Zatorska, D., Kim, J., Aguirre, J., Llauger, L., She, Y., Wu, N., Immormino, R. M., Gewirth, D. T., and Chiosis, G. (2006). Identification of potent water soluble purine-scaffold inhibitors of the heat shock protein 90. J Med Chem 49, 381-390.
Holzbeierlein, J., Lal, P., LaTulippe, E., Smith, A., Satagopan, J., Zhang, L., Ryan, C., Smith, S., Scher, H., Scardino, P., et al. (2004). Gene expression analysis of human prostate carcinoma during hormonal therapy identifies androgen-responsive genes and mechanisms of therapy resistance. Am J Pathol 164, 217-227.
Hughes, T. R., Marton, M. J., Jones, A. R., Roberts, C. J., Stoughton, R., Armour, C. D., Bennett, H. A., Coffey, E., Dai, H., He, Y. D., et al. (2000). Functional discovery via a compendium of expression profiles. Cell 102, 109-126.
Kim, J., Felts, S., Llauger, L., He, H., Huezo, H., Rosen, N., and Chiosis, G. (2004). Development of a fluorescence polarization assay for the molecular chaperone Hsp90. J Biomol Screen 9, 375-381.
Kovacs, J. J., Murphy, P. J., Gaillard, S., Zhao, X., Wu, J. T., Nicchitta, C. V., Yoshida, M., Toft, D. O., Pratt, W. B., and Yao, T. P. (2005). HDAC6 regulates Hsp90 acetylation and chaperone-dependent activation of glucocorticoid receptor. Mol Cell 18, 601-607.
Lam, J. S., Leppert, J. T., Vemulapalli, S. N., Shvarts, O., and Belldegrun, A. S. (2006). Secondary hormonal therapy for advanced prostate cancer. J Urol 175, 27-34.
Lamb, J., Crawford, E. D., Peck, D., Modell, J. W., Blat, I. C., Wrobel, M. J., Lerner, J., Brunet, J.-P., Subramanian, A., Ross, K. N., et al. (2006). The Connectivity Map: Using Gene-expression Signatures to Connect Small Molecules, Genes and Disease. Science in press.
Lamb, J., Ramaswamy, S., Ford, H. L., Contreras, B., Martinez, R. V., Kittrell, F. S., Zahnow, C. A., Patterson, N., Golub, T. R., and Ewen, M. E. (2003). A mechanism of cyclin D1 action encoded in the patterns of gene expression in human cancer. Cell 114, 323-334.
Linja, M. J., Savinainen, K. J., Saramaki, O. R., Tammela, T. L., Vessella, R. L., and Visakorpi, T. (2001). Amplification and overexpression of androgen receptor gene in hormone-refractory prostate cancer. Cancer Res 61, 3550-3555.
Llauger-Bufi, L., Felts, S. J., Huezo, H., Rosen, N., and Chiosis, G. (2003). Synthesis of novel fluorescent probes for the molecular chaperone Hsp90. Bioorg Med Chem Lett 13, 3975-3978.
MacKinnon, S., Durst, T., Arnason, J. T., Angerhofer, C., Pezzuto, J., Sanchez-Vindas, P. E., Poveda, L. J., and Gbeassor, M. (1997). Antimalarial activity of tropical Meliaceae extracts and gedunin derivatives. J Nat Prod 60, 336-341.
Majumder, P. K., and Sellers, W. R. (2005). Akt-regulated pathways in prostate cancer. Oncogene 24, 7465-7474.
Marcu, M. G., Schulte, T. W., and Neckers, L. (2000). Novobiocin and related, coumarins and depletion of heat shock protein 90-dependent signaling proteins. J Natl Cancer Inst 92, 242-248.
Mimnaugh, E. G., Xu, W., Vos, M., Yuan, X., Isaacs, J. S., Bisht, K. S., Gius, D., and Neckers, L. (2004). Simultaneous inhibition of hsp 90 and the proteasome promotes protein ubiquitination, causes endoplasmic reticulum-derived cytosolic vacuolization, and enhances antitumor activity. Mol Cancer Ther 3, 551-566.
Murakami, Y., Uehara, Y., Yamamoto, C., Fukazawa, H., and Mizuno, S. (1991). Induction of hsp 72/73 by herbimycin A, an inhibitor of transformation by tyrosine kinase oncogenes. Exp Cell Res 195, 338-344.
Padma, T. V. (2005). Ayurveda. Nature 436, 486.
Parsons, A. B., Brost, R. L., Ding, H., Li, Z., Zhang, C., Sheikh, B., Brown, G. W., Kane, P. M., Hughes, T. R., and Boone, C. (2004). Integration of chemical-genetic and genetic interaction data links bioactive compounds to cellular target pathways. Nat Biotechnol 22, 62-69.
Peck, D., Crawford, E. D., Ross, K. N., Stegmaier, K., Golub, T. R., and Lamb, J. (2006). A Method for High-Throughput Gene Expression Signature Analysis. Genome Biol 7, R61.
Reich, M., Liefeld, T., Gould, J., Lerner, J., Tamayo, P., and Mesirov, J. P. (2006). GenePattern 2.0. Nat Genet. 38, 500-501.
Soti, C., Racz, A., and Csermely, P. (2002). A Nucleotide-dependent molecular switch controls ATP binding at the C-terminal domain of Hsp90. N-terminal nucleotide binding unmasks a C-terminal binding pocket. J Biol Chem 277, 7066-7075.
Stegmaier, K., Ross, K. N., Colavito, S. A., O'Malley, S., Stockwell, B. R., and Golub, T. R. (2004). Gene expression-based high-throughput screening (GE-HTS) and application to leukemia differentiation. Nat Genet 36, 257-263.
Sun, H., Liu, X., Xiong, Q., Shikano, S., and Li, M. (2006). Chronic inhibition of cardiac kir2.1 and HERG potassium channels by celastrol with dual effects on both ion conductivity and protein trafficking. J Biol Chem 281, 5877-5884.
Ushiro, S., Ono, M., Nakayama, J., Fujiwara, T., Komatsu, Y., Sugimachi, K., and Kuwano, M. (1997). New nortriterpenoid isolated from anti-rheumatoid arthritic plant, Tripterygium wilfordii, modulates tumor growth and neovascularization. Int J Cancer 72, 657-663.
Vilenchik, M., Solit, D., Basso, A., Huezo, H., Lucas, B., He, H., Rosen, N., Spampinato, C., Modrich, P., and Chiosis, G. (2004). Targeting wide-range oncogenic transformation via PU24FCl, a specific inhibitor of tumor Hsp90. Chem Biol 11, 787-797.
Visakorpi, T., Hyytinen, E., Koivisto, P., Tanner, M., Keinanen, R., Palmberg, C., Palotie, A., Tammela, T., Isola, J., and Kallioniemi, O. P. (1995). In vivo amplification of the androgen receptor gene and progression of human prostate cancer. Nat Genet. 9, 401-406.
Walsh, P. C. (1996). Treatment of benign prostatic hyperplasia. N Engl J Med 335, 586-587.
Wang, J., Gines, S., MacDonald, M. E., and Gusella, J. F. (2005). Reversal of a full-length mutant huntingtin neuronal cell phenotype by chemical inhibitors of polyglutamine-mediated aggregation. BMC Neurosci 6, 1.
Waza, M., Adachi, H., Katsuno, M., Minamiyama, M., Sang, C., Tanaka, F., Inukai, A., Doyu, M., and Sobue, G. (2005). 17-AAG, an Hsp90 inhibitor, ameliorates polyglutamine-mediated motor neuron degeneration. Nat Med 11, 1088-1095.
Westerheide, S. D., Bosman, J. D., Mbadugha, B. N., Kawahara, T. L., Matsumoto, G., Kim, S., Gu, W., Devlin, J. P., Silverman, R. B., and Morimoto, R. I. (2004). Celastrols as inducers of the heat shock response and cytoprotection. J Biol Chem 279, 56053-56060.
Whitesell, L., and Lindquist, S. L. (2005). HSP90 and the chaperoning of cancer. Nat Rev Cancer 5, 761-772.

Yang, H., Chen, D., Cui, Q. C., Yuan, X., and Dou, Q. P. (2006). Celastrol, a triterpene extracted from the Chinese “Thunder of God Vine,” is a potent proteasome inhibitor and suppresses human prostate cancer growth in nude mice. Cancer Res 66, 4758-4765.
Zhao, R., Davey, M., Hsu, Y. C., Kaplanek, P., Tong, A., Parsons, A. B., Krogan, N., Cagney, G., Mai, D., Greenblatt, J., et al. (2005). Navigating the chaperone network: an integrative map of physical and genetic interactions mediated by the hsp90 chaperone. Cell 120, 715-727.

### Example 2

### Identification of Gedunin as an HSP90 Inhibitor Using the Connectivity Map

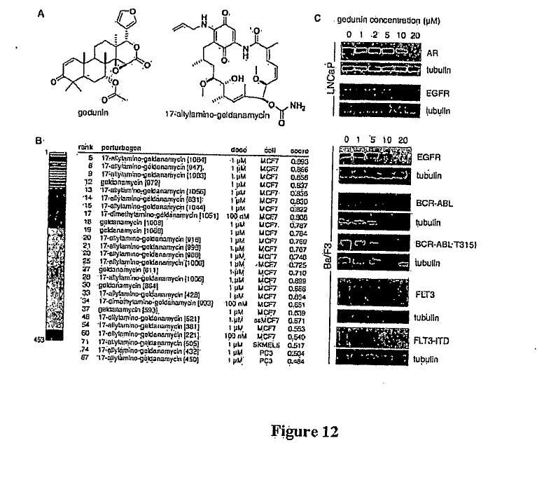

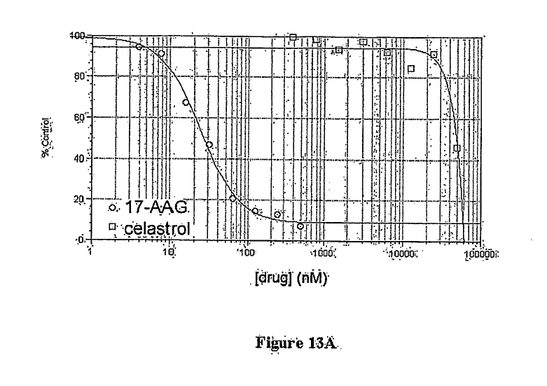

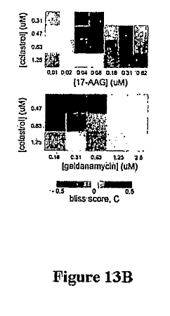

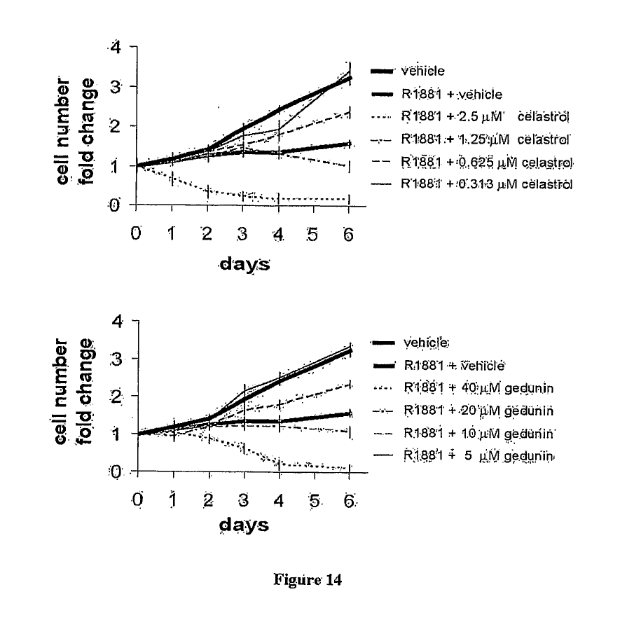

The Connectivity Map (as described in Lamb et al., Science 313:1929-1935, 29 Sep. 2006, incorporated herein by reference) has been used to generate hyoptheses about the mechanism of action of uncharacterized small molecules. As described in Example 1, we performed a high-throughput gene expression-based screen for small molecules capable of abrogating the gene-expression signature of androgen receptor (AR) activation in prostate cancer cells. One of the hits from the screen was the triterpenoid natural product gedunin (Khalid et al. Nat. Prod. 52:922, 1989; incorporated herein by reference) (FIG. 12A), purified from the Meliacae family of medicinal plants. The mechanism by which gedunin abrogated AR activity was entirely unknown because this compound has not been extensively characterized.

In an effort to elucidate its mechanism of action, we defined a signature from gedunin by treating LNCaP prostate cancer cells for 6 hours with the compound, and queried the Connectivity Map. High connectivity scores were found for multiple instances of three heat shock protein 90 (HSP90) inhibitors: geldanamycin, 17-allylamino-geldanamycin, and 17-dimethylamino-geldanamycin (FIG. 12B). As a class, these HSP90 inhibitors showed marked connectivity to the gedunin signature (permutation P-value <0.0001).

This result suggests that gedunin, though structurally dissimilar from known HSP90 inhibitors (FIG. 12A), might impinge upon the HSP90 pathway. Because the stability of AR is known to be dependent upon HSP90 activity, we asked whether AP expression could be diminished by gedunin treatment. Immunoblotting indicated that AR protein, as well as other HSP90-interacting proteins, was nearly entirely eliminated in gedunin-treated LNCaP and Ba/F3 cells (FIG. 12C), consistent with gedunin as an inhibitor of HSP90 function. Moreover, mutant interacting proteins such as the BCR-ABL T315I point mutant and the FLT3 internal tandem duplication (ITD) mutant show increased sensitivity to gedunin-mediated inhibition, as is seen upon HSP90 inhibition by geldanamycins (Gorre et al. Blood 100:3041, 2002; Yao et al. Clin. Cancer Res. 9:4483, 2003; each of which is incorporated herein by reference). Further biochemical studies demonstrated that the mechanism of abrogating HSP90 function was distinct from geldanamycin and its analogs.

### Example 3

### Celastrol and Gedunin Inhibit Cancer Cell Growth

We also addressed whether celastrol and gedunin activity results in decreased cell growth consistent with inhibition of AR and other HSP90 clients. Both compound inhibit LNCaP cell viability in the presence of androgen and mimicking the growth inhibition effects of androgen deprivation around their EC₅₀ of androgen signaling inhibition (FIG. 9). Below this concentration, an intermediate viability inhibition is seen. At higher concentrations, both compounds markedly decrease LNCaP prostate cancer cell viability and induce apoptosis. They similarly induct apoptosis in the SKBR breast cancer cell line, though to a greater extent than seen with existing HSP90 ATP binding site inhibitors.

### Example 4

### Celastrol Binds Purified HSP90

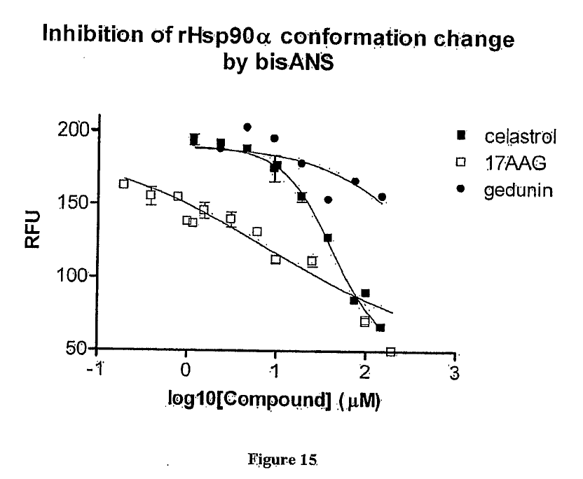

The HSP90 conformational change assay is based on a previously published method that uses the fluorophore 1,1′-bis(4-anilino-5-naphthalenesulfonic acid (bis-ANS) (Invitrogen #B-153) to measure Grp94 conformational changes. HSP90 (Stressgen, BC, Canada) at a final concentration of 200 nM in buffer A (110 mM KOAc, 20 mM NaCl, 2 mM Mg(OAc)₂, 25 mM K-HEPES, pH 7.2, 100 μM CaCl₂) was added to each well of a 96-well plate. Test compounds or a DMSO control were added to a well an the indicated concentration, and the plates were mixed for 30 s on a plate shaker before incubation for 60 min. at 37° C. Then to each well, bis-ANS was added to yield a final concentration of 50 μM. The final volume in each well was 100 μL. The plate was covered with foil and mixed for 30 s on a plate shaker before incubation for 60 min. at 37° C. Relative fluorescence units were measured using a SpectraMx Gemini XS spectrofluorometer (Molecular Devices Corporation, Sunnyvale, Calif.) at an excitation wavelength for bis-ANS of 393 nm and an emission wavelength of 484 nm. The data were acquired using the SOFTmaxPRO software (Molecular Devices Corporation, Sunnyvale, Calif.). The background was defined as the RFU generated from wells that did not contain HSP90 but to which bis-ANS was added. IC₅₀ was defined as the concentration of the compound at which there was 50% inhibition of bis-ANS activity. As shown in FIG. 15, celastrol binds purified HSP90 in vitro as indicated by celastrol's ability to inhibit HSP90 conformational changes induced by bis-ANS. The effect of celastrol is observed at moderate concentrations, but the effect is not well seen for gedunin, possibly due to the relatively high concentrations needed to observe gedunin's effect.

1. A method of inhibiting Hsp90 protein activity, the method comprising steps of: contacting Hsp90 protein with an amount of celastrol, gedunin, or a derivative or salt of celastrol or gedunin sufficient to inhibit the activity of Hsp90 protein.

2. The method of claim 1, wherein the step of contacting is performed in cell culture.

3. (canceled)

4. The method of claim 1, wherein the step of contacting is performed in a subject.

5. The method of claim 1, wherein the step of contacting is performed in vitro.

6. The method of claim 1, wherein the step of contacting is performed in vivo.

7. The method of claim 1, wherein the Hsp90 protein is purified Hsp90 protein.

8. The method of claim 1, wherein the Hsp90 protein is unpurified Hsp90 protein.

9. (canceled)

10. The method of claim 1, wherein the step of contacting comprises contacting Hsp90 protein with celastrol.

11. The method of claim 1, wherein the step of contacting comprises contacting Hsp90 protein with celastrol derivative selected from the group consisting of dihydrocelastrol, pristimerol, dihydrocelastrol diacetate, celastrol methyl ester, celastrol benzyl ester, celastrol butyl ester, pristimerol diacetate, and celastrol triacetate.

12. The method of claim 1, wherein the step of contacting comprises contacting Hsp90 protein with a celastrol derivative of the formula: 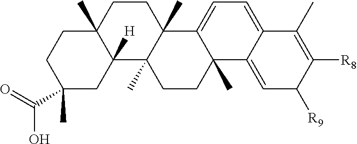
wherein
R₈ is hydroxyl (—OH) or acetyl-protected hydroxyl 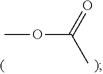
and
R₉ is oxo (═O), hydrogen (—H), or acetyl-protected hydroxyl

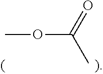

13. The method of claim 1, wherein the step of contacting comprises contacting Hsp90 protein with gedunin.

14. The method of claim 1, wherein the step of contacting comprises contacting Hsp90 protein with a derivate of gedunin selected from the group consisting of deoxygedunin, deacetylgedunin, 7-desacetoxy-6,7-dehydrogedunin, 3-deoxo-3beta-acetoxydeoxydihydrogedunin, deacetoxy-7-oxogedunin, deacetylgedunin, dihydro-7-desacetyaldeoxygedunin, and 3alpha-hydroxydeoxodihydrogedunin.

15. The method of claim 1, wherein the step of contacting comprises contacting Hsp90 protein with a derivative of gedunin of the formula: 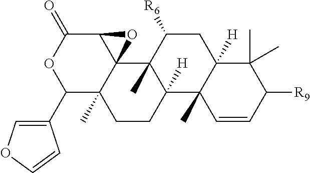
wherein
R₆ is hydrogen (—H); oxo (═O), hydroxyl (—OH), or acetyl-protected hydroxyl 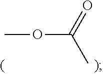
and
R₉ is oxo (═O), or acetyl-protected hydroxyl

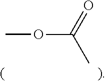

16. The method of claim 1 further comprising contact Hsp90 with at least one other Hsp90 inhibitor.

17. The method of claim 16, wherein the other Hsp90 inhibitor is selected from the group consisting of geldanamycin, 17-AAG, monorden (a.k.a., radicicol), IPI-504, DMAG, and novobiocin.

18. The method of claim 1, wherein inhibiting the activity of Hsp90 destabilizes androgen receptors.

19. The method of claim 1, wherein inhibiting the activity of Hsp90 destabilizes glucocorticoid receptors.

20. The method of claim 1, wherein inhibiting the activity of Hsp90 destabilizes oncogenes.

21. A method of treating a subject with cancer, the method comprising steps of:
administering to a subject with cancer a therapeutically effective amount of celastrol, gedunin, or a salt or derivative of celastrol or gedunin.

22. (canceled)

23. The method of claim 21, wherein the subject is human.

24. The method of claim 21, wherein the cancer is prostate cancer.

25. The method of claim 24, wherein the prostate cancer is dependent upon androgen receptor signaling.

26. The method of claim 21, wherein the cancer is breast cancer.

27. The method of claim 26, wherein the breast cancer is dependent upon estrogen or progesterone receptor signalling.

28. The method of claim 21, wherein the cancer is leukemia.

29. The method of claim 28, wherein the leukemia is BCR/ABL chronic myeloid leukemia or an FLT3 mutant leukemia.

30. The method of claim 21, wherein the cancer is lung cancer.

31. The method of claim 30, wherein the lung cancer is an EGFR mutant cancer.

32. The method of claim 21, wherein the cancer is colon cancer.

33. The method of claim 21, wherein the cancer is ovarian cancer.

34. The method of claim 21, wherein the cancer is an AKT mutant cancer.

35. The method of claim 21, wherein the cancer is driven by a mutated protein kinase.

36. The method of claim 21, wherein the cancer is driven by a nuclear hormone receptor.

37. The method of claim 21, wherein the step of administering comprises administering to the subject with cancer a therapeutically effective amount of celastrol.

38. The method of claim 11, wherein the step of administering comprises administering to the subject with cancer a therapeutically effective amount of a celastrol derivative selected from the group consisting of dihydrocelastrol, pristimerol, dihydrocelastrol diacetate, celastrol methyl ester, celastrol benzyl ester, celastrol butyl ester, pristimerol diacetate, and celastrol triacetate.

39. The method of claim 21, wherein the step of administering comprises administering to the subject with cancer a therapeutically effective amount of a celastrol derivative of the formula: 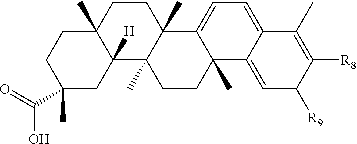
wherein
R₈ is hydroxyl (—OH) or acetyl-protected hydroxyl 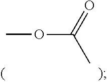
and
R₉ is oxo (═O), hydrogen (—H), or acetyl-protected hydroxyl

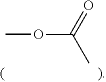

40. The method of claim 21, wherein the step of administering comprises administering to the subject with cancer a therapeutically effective amount of gedunin.

41. The method of claim 21, wherein the step of administering comprises administering to the subject with cancer a therapeutically effective amount of a derivate of gedunin selected from the group consisting of deoxygedunin, deacetylgedunin, 7-desacetoxy-6,7-dehydrogedunin, 3-deoxo-3beta-acetoxydeoxydihydrogedunin, deacetoxy-7-oxogedunin, deacetylgedunin, dihydro-7-desacetyaldeoxygedunin, and 3alpha-hydroxydeoxodihydrogedunin.

42. The method of claim 21, wherein the step of administering comprises administering to the subject with cancer a therapeutically effective amount of a derivative of gedunin of the formula: 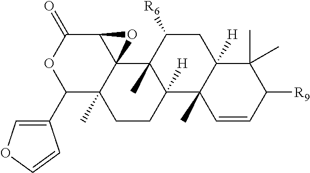
wherein
R₆ is hydrogen (—H); oxo (═O), hydroxyl (—OH), or acetyl-protected hydroxyl 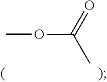
and
R₉ is oxo (═O), or acetyl-protected hydroxyl

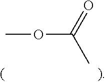

43. (canceled)

44. A compound of formula: 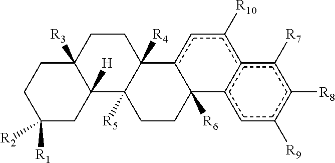
wherein
each dashed line independently represents either the presence or absence of a bond;
R₁ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{A}; —C(═O)R_{A}; —CHO; —CO₂H; —CO₂R_{A}; —CN; —SCN; —SR_{A}; —SOR_{A}; —SO₂R_{A}; —NO₂; —N₃; —NH₂; —NHR_{A}; —N(R_{A})₂; —NHC(═O)R_{A}; —NR_{A}C(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₂ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{B}; —C(═O)R_{B}; —CHO; —CO₂H; —CO₂R_{B}; —CN; —SCN; —SR_{B}; —SOR_{B}; —SO₂R_{B}; —NO₂; —N₃; —NH₂; —NHR_{B}; —N(R_{B})₂; —NHC(═O)R_{B}; —NR_{B}C(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₃ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{C}; —C(═O)R_{C}; —CHO; —CO₂H; —CO₂R_{C}; —CN; —SCN; —SR_{C}; —SOR_{C}; —SO₂R_{C}; —NO₂; —N₃; —NH₂; —NHR_{C}; —N(R_{C})₂; —NHC(═O)R_{C}; —NR_{C}C(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₄ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{D}; —C(═O)R_{D}; —CHO; —CO₂H; —CO₂R_{D}; —CN; —SCN; —SR_{D}; —SOR_{D}; —SO₂R_{D}; —NO₂; —N₃; —NH₂; —NHR_{D}; —N(R_{D})₂; —NHC(═O)R_{D}; —NR_{D}C(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₅ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{E}; —C(═O)R_{E}; —CHO; —CO₂H; —CO₂R_{E}; —CN; —SCN; —SR_{E}; —SOR_{E}; —SO₂R_{E}; —NO₂; —N₃; —NH₂; —NHR_{E}; —N(R_{E})₂; —NHC(═O)R_{E}; —NR_{E}C(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₆ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{F}; —C(═O)R_{F}; —CHO; —CO₂H; —CO₂R_{F}; —CN; —SCN; —SR_{F}; —SOR_{F}; —SO₂R_{F}; —NO₂; —N₃; —NH₂; —NHR_{F}; —N(R_{F})₂; —NHC(═O)R_{F}; —NR_{F}C(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₇ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{G}; ═O; —C(═O)R_{G}; —CHO; —CO₂H; —CO₂R_{G}; —CN; —SCN; —SR_{G}; —SOR_{G}; —SO₂R_{G}; —NO₂; —N₃; —NH₂; —NHR_{G}; —N(R_{G})₂; —NHC(═O)R_{G}; —NR_{G}C(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₈ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{H}; ═O; —C(═O)R_{H}; —CHO; —CO₂H; —CO₂R_{H}; —CN; —SCN; —SR_{H}; —SOR_{H}; —SO₂R_{H}; —NO₂; —N₃; —NH₂; —NHR_{H}; —N(R_{H})₂; —NHC(═O)R_{H}; —NR_{H}C(═O)R_{H}; —NR_{H}C(═O)N(R_{H})₂; —OC(═O)OR_{H}; —OC(═O)R_{H}; —OC(═O)N(R_{H})₂; —NR_{H}C(═O)OR_{H}; or —C(R_{H})₃; wherein each occurrence of R_{H} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₉ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{I}; ═O; —C(═O)R_{I}; —CHO; —CO₂H; —CO₂R_{I}; —CN; —SCN; —SR_{I}; —SOR_{I}; —SO₂R_{I}; —NO₂; —N₃; —NH₂; —NHR_{I}; —N(R_{I})₂; —NHC(═O)R_{I}; —NR_{I}C(═O)R_{I}; —NR_{I}C(═O)N(R_{I})₂; —OC(═O)OR_{I}; —OC(═O)R_{I}; —OC(═O)N(R_{I})₂; —NR_{I}C(═O)OR_{I}; or —C(R_{I})₃; wherein each occurrence of R_{I} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₁₀ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{J}; ═O; —C(═O)R_{J}; —CHO; —CO₂H; —CO₂R_{J}; —CN; —SCN; —SR_{J}; —SOR_{J}; —SO₂R_{J}; —NO₂; —N₃; —NH₂; —NHR_{I}; —N(R_{J})₂; —NHC(═O)R_{J}; —NR_{J}C(═O)R_{J}; —NR_{J}C(═O)N(R_{J})₂; —OC(═O)OR_{J}; —OC(═O)R_{J}; —OC(═O)N(R_{J})₂; —NR_{I}C(═O)OR_{J}; or —C(R_{J})₃; wherein each occurrence of R_{J} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety; and pharmaceutically acceptable salts, stereoisomers, tautomers, and pro-drugs thereof.

45-56. (canceled)

57. A compound of formula: 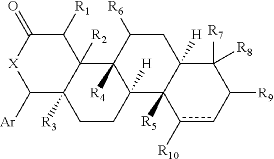
wherein
Ar is a substituted or unsubstituted aryl or heteroaryl moiety;
X is —O—, —NH—, —NR_{X}—, —CH₂—, —CHR_{X}—, or —C(R_{X})₂—, wherein R_{X} is a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; heteroaryloxy; or heteroarylthio moiety;
a dashed line represents either the presence or absence of a bond;
R₁ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{A}; —C(═O)R_{A}; —CHO; —CO₂H; —CO₂R_{A}; —CN; —SCN; —SR_{A}; —SOR_{A}; —SO₂R_{A}; —NO₂; —N₃; —NH₂; —NHR_{A}; —N(R_{A})₂; —NHC(═O)R_{A}; —NR_{A}C(═O)R_{A}; —NR_{A}C(═O)N(R_{A})₂; —OC(═O)OR_{A}; —OC(═O)R_{A}; —OC(═O)N(R_{A})₂; —NR_{A}C(═O)OR_{A}; or —C(R_{A})₃; wherein each occurrence of R_{A} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₂ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{B}; —C(═O)R_{B}; —CHO; —CO₂H; —CO₂R_{B}; —CN; —SCN; —SR_{B}; —SOR_{B}; —SO₂R_{B}; —NO₂; —N₃; —NH₂; —NHR_{B}; —N(R_{B})₂; —NHC(═O)R_{B}; —NR_{B}C(═O)R_{B}; —NR_{B}C(═O)N(R_{B})₂; —OC(═O)OR_{B}; —OC(═O)R_{B}; —OC(═O)N(R_{B})₂; —NR_{B}C(═O)OR_{B}; or —C(R_{B})₃; wherein each occurrence of R_{B} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₁ and R₂ may be taken together to form an epoxide ring, aziridine ring, cyclopropyl ring, or a bond of a carbon-carbon double bond;
R₃ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{C}; —C(═O)R_{C}; —CHO; —CO₂H; —CO₂R_{C}; —CN; —SCN; —SR_{C}; —SOR_{C}; —SO₂R_{C}; —NO₂; —N₃; —NH₂; —NHR_{C}; —N(R_{C})₂; —NHC(═O)R_{C}; —NR_{C}C(═O)R_{C}; —NR_{C}C(═O)N(R_{C})₂; —OC(═O)OR_{C}; —OC(═O)R_{C}; —OC(═O)N(R_{C})₂; —NR_{C}C(═O)OR_{C}; or —C(R_{C})₃; wherein each occurrence of R_{C} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₄ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{D}; —C(═O)R_{D}; —CHO; —CO₂H; —CO₂R_{D}; —CN; —SCN; —SR_{D}; —SOR_{D}; —SO₂R_{D}; —NO₂; —N₃; —NH₂; —NHR_{D}; —N(R_{D})₂; —NHC(═O)R_{D}; —NR_{D}C(═O)R_{D}; —NR_{D}C(═O)N(R_{D})₂; —OC(═O)OR_{D}; —OC(═O)R_{D}; —OC(═O)N(R_{D})₂; —NR_{D}C(═O)OR_{D}; or —C(R_{D})₃; wherein each occurrence of R_{D} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₅ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{E}; —C(═O)R_{E}; —CHO; —CO₂H; —CO₂R_{E}; —CN; —SCN; —SR_{E}; —SOR_{E}; —SO₂R_{E}; —NO₂; —N₃; —NH₂; —NHR_{E}; —N(R_{E})₂; —NHC(═O)R_{E}; —NR_{E}C(═O)R_{E}; —NR_{E}C(═O)N(R_{E})₂; —OC(═O)OR_{E}; —OC(═O)R_{E}; —OC(═O)N(R_{E})₂; —NR_{E}C(═O)OR_{E}; or —C(R_{E})₃; wherein each occurrence of R_{E} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₆ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{F}; —C(═O)R_{F}; —CHO; —CO₂H; —CO₂R_{F}; —CN; —SCN; —SR_{F}; —SOR_{F}; —SO₂R_{F}; —NO₂; —N₃; —NH₂; —NHR_{F}; —N(R_{F})₂; —NHC(═O)R_{F}; —NR_{F}C(═O)R_{F}; —NR_{F}C(═O)N(R_{F})₂; —OC(═O)OR_{F}; —OC(═O)R_{F}; —OC(═O)N(R_{F})₂; —NR_{F}C(═O)OR_{F}; or —C(R_{F})₃; wherein each occurrence of R_{F} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₇ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{G}; —C(═O)R_{G}; —CHO; —CO₂H; —CO₂R_{G}; —CN; —SCN; —SR_{G}; —SOR_{G}; —SO₂R_{G}; —NO₂; —N₃; —NH₂; —NHR_{G}; —N(R_{G})₂; —NHC(═O)R_{G}; —NR_{G}C(═O)R_{G}; —NR_{G}C(═O)N(R_{G})₂; —OC(═O)OR_{G}; —OC(═O)R_{G}; —OC(═O)N(R_{G})₂; —NR_{G}C(═O)OR_{G}; or —C(R_{G})₃; wherein each occurrence of R_{G} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₈ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{H}; —C(═O)R_{H}; —CHO; —CO₂H; —CO₂R_{H}; —CN; —SCN; —SR_{H}; —SOR_{H}; —SO₂R_{H}; —NO₂; —N₃; —NH₂; —NHR_{H}; —N(R_{H})₂; —NHC(═O)R_{H}; —NR_{H}C(═O)R_{H}; —NR_{H}C(═O)N(R_{H})₂; —OC(═O)OR_{H}; —OC(═O)R_{H}; —OC(═O)N(R_{H})₂; —NR_{H}C(═O)OR_{H}; or —C(R_{H})₃; wherein each occurrence of R_{H} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₉ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{I}; ═O; —C(═O)R_{I}; —CHO; —CO₂H; —CO₂R_{I}; —CN; —SCN; —SR_{I}; —SOR_{I}; —SO₂R_{I}; —NO₂; —N₃; —NH₂; —NHR_{I}; —N(R_{I})₂; —NHC(═O)R_{I}; —NR_{I}C(═O)R_{I}; —NR_{I}C(═O)N(R_{I})₂; —OC(═O)OR_{I}; —OC(═O)R_{I}; —OC(═O)N(R_{I})₂; —NR_{I}C(═O)OR_{I}; or —C(R_{I})₃; wherein each occurrence of R_{I} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety;
R₁₀ is selected from the group consisting of hydrogen; halogen; cyclic or acyclic, substituted or unsubstituted, branched or unbranched aliphatic; cyclic or acyclic, substituted or unsubstituted, branched or unbranched heteroaliphatic; substituted or unsubstituted, branched or unbranched acyl; substituted or unsubstituted, branched or unbranched aryl; substituted or unsubstituted, branched or unbranched heteroaryl; —OH; —OR_{J}; ═O; —C(═O)R_{J}; —CHO; —CO₂H; —CO₂R_{J}; —CN; —SCN; —SR_{J}; —SOR_{J}; —SO₂R_{J}; —NO₂; —N₃; —NH₂; —NHR_{I}; —N(R_{J})₂; —NHC(═O)R_{J}; —NR_{J}C(═O)R_{J}; —NR_{J}C(═O)N(R_{J})₂; —OC(═O)OR_{J}; —OC(═O)R_{J}; —OC(═O)N(R_{J})₂; —NR_{I}C(═O)OR_{J}; or —C(R_{J})₃; wherein each occurrence of R_{J} is independently a hydrogen, a halogen, a protecting group, an aliphatic moiety, a heteroaliphatic moiety, an acyl moiety; an aryl moiety; a heteroaryl moiety; hydroxy, alkoxy; aryloxy; thioxy; alkylthio; arylthio; amino, alkylamino, dialkylamino, heteroaryloxy; or heteroarylthio moiety; and pharmaceutically acceptable salts, stereoisomers, tautomers, and pro-drugs thereof.

58-83. (canceled)

84. A method of inhibiting Hsp90 protein activity, the method comprising steps of:
contacting Hsp90 protein with an amount of a compound of claim 44 sufficient to inhibit the activity of Hsp90 protein.

85. A method of destabilizing a receptor, the method comprising steps of:
contacting a cell with an amount of a compound of claim 44 sufficient to destabilize glucocorticoid receptors in the cell.

86. A method of inhibiting receptor signaling, the method comprising steps of:
contacting a cell with an amount of a compound of claim 44 sufficient to inhibit receptor signaling.

87. The method of claim 85, wherein the receptor is a glucocorticoid receptor.

88. The method of claim 87, wherein the glucocorticoid receptor is an androgen receptor.

89. The method of claim 87, wherein the glucocorticoid receptor is an estrogen receptor.

90. The method of claim 85, wherein the receptor is epidermal growth factor receptor (EGFR).

91. A method of destabilizing oncogenic proteins, the method comprising steps of:
contacting a cell with an amount of a compound of claim 44 sufficient to destablize oncogenic proteins in the cell.

92. The method of claim 91, wherein the oncogenic protein is selected from the group consisting of p53, Bcr-Abl, Her2, Akt, FLT3, v-src, casein kinase II, and Raf-1.

93. A method of treating a subject with cancer, the method comprising steps of:
administering to a subject with cancer a therapeutically effective amount of a compound of claim 44.

94. (canceled)

95. A pharmaceutical composition comprising (1) celastrol, gedunin, or a salt or derivative thereof; and (2) a pharmaceutically acceptable excipient.

96. The pharmaceutical composition of claim 95 comprising (1) celastrol; and (2) a pharmaceutically acceptable excipient.

97. The pharmaceutical composition of claim 95, wherein celastrol or a derivative thereof is of formula: 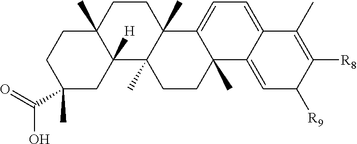
wherein
R₈ is hydroxyl (—OH) or acetyl-protected hydroxyl 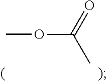
and
R₉ is oxo (═O), hydrogen (—H), or acetyl-protected hydroxyl

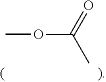

98. The pharmaceutical composition of claim 95 comprising (1) gedunin; and (2) a pharmaceutically acceptable excipient.

99. The pharmaceutical composition of claim 95, wherein gedunin or a derivative thereof is of formula: 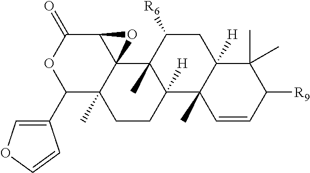
wherein
R₆ is hydrogen (—H); oxo (═O), hydroxyl (—OH), or acetyl-protected hydroxyl 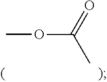
and
R₉ is oxo (═O), or acetyl-protected hydroxyl

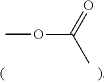

100. A pharmaceutical composition comprising a compound of claim 44 and a pharmaceutically acceptable excipient.

101. The pharmaceutical composition of claim 95 further comprising a cytotoxic agent.

102. The pharmaceutical composition of claim 95 further comprising an anti-cancer agent.

103. The pharmaceutical composition of claim 95 further comprising an Hsp90 inhibitor.

104. The pharmaceutical composition of claim 103, wherein the Hsp90 inhibitor is selected from the group consisting of geldanamycin, 17-AAG, monorden (a.k.a., radicicol), IPI-504, DMAG, and novobiocin.
